SEQUENCE LISTING (7 sequences)

SEQ ID NO: 1 - DNA, 1509 bp, Faecalibacterium prausnitzii Strain A
tataaagagtttgatcctggctcaggacgaacgctggcggcgcgcctaacacatgcaagtcgaacgagcgagagagagcttgctttctcgagtgagtggcgaacgggtgagtaacgcgtgaggaacctgcctcaaagagggggacaacagttggaaacgactgctaataccgcataagcccacgacccggcatcgggtagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgcgacggtgctggaaacagtactttccttcgggacgcagagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgcagatcagcatgctgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtagccgtaggagaacctgcggctggatcacctccttt

SEQ ID NO: 2 - DNA, 1526 bp, Fournierella massiliensis strain A
tatgaagagtttgatcctggctcaggacgaacgctggcggcgcgcctaacacatgcaagtcgaacggagcttgcttgtcagatcctttcggggtgacgacttgtaagcttagtggcgaacgggtgagtaacacgtgagtaacctgccccagagtgggggacaacagttggaaacgactgctaataccgcataagcccacggaaccgcatggttctgagggaaaaggagcaattcgctttgggatggactcgcgtccgattagctagatggtgaggtaacggcccaccatggcgacgatcggtagccggactgagaggttgatcggccacattgggactgagacacggcccagactcctacgggaggcagcagtggggaatattgcacaatgggggaaaccctgatgcagcgacgccgcgtggaggaagaaggccttcgggttgtaaactcctgtcgtaagggacgatagtgacggtaccttacaagaaagccacggctaactacgtgccagcagccgcggtaaaacgtaggtggcaagcgttgtccggaattactgggtgtaaagggagcgcaggcgggtctgcaagttggaagtgaaactcatgggctcaacccatgaactgctttcaaaactgcggatcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtgggaggattgaccccttccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagtgtagagatacatgaagtccttcgggacatcgagacaggtggtgcatggttgtcgtcagctcgtgtcgtgagatgttgggttaagtcccgcaacgagcgcaacccttatcgttagttactacgcaagaggactctagcgagactgccgttgacaaaacggaggaaggtggggatgacgtcaaatcatcatgccctttatgacctgggctacacacgtactacaatggcaattaacaaagagaagcgaagccgcgaggtggagcaaacctcataaaaattgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtccgtagcctaaccgcaaggagggcgcggccgaaggtaaaactggtgattggggtgaagtcgtaacaaggtagccgtatcggaaggtgcggctggatcacctccttt

SEQ ID NO: 3 - DNA, 1511 bp, Harryflintia acetispora strain A
ctaaagagtttgatcctggctcaggacgaacgctggcggcgcgcctaacacatgcaagtcgaacggagaaatgctgagcttgctttgcatttcttagtggcggacgggtgagtaacacgtgagcaacctgcctttgtgaggggaataacgtctggaaacggacgctaataccgcataacgtcaaggaaccgcatggttttttgaccaaagattttatcgcacaaagatgggctcgcggctgattagctagttggcggggtaacggcccaccaaggcgacgatcagtagccggactgagaggttgatcggccacattgggactgagacacggcccagactcctacgggaggcagcagtgggggatattgcacaatgggggaaaccctgatgcagcgacgccgcgtgagggaagacggttttcggattgtaaacctctgtcttcagggacgaaatcaatgacggtacctgaggaggaagccacggctaactacgtgccagcagccgcggtaatacgtaggtggcaagcgttgtccggaattactgggtgtaaagggagcgtaggcgggaatgcaagttgaatgtttaaactatcggctcaactgataatcgcgttcaaaactgcatttcttgagtggagtagaggcaggcggaattcctagtgtagcggtgaaatgcgtagatattaggaggaacaccagtggcgaaggcggcctgctgggctctaactgacgctgaggctcgaaagcgtgggtagcaaacaggattagataccctggtagtccacgccgtaaacgatgattactaggtgtggggggactgaccccttccgtgccggagttaacacaataagtaatccacctggggagtacggtcgcaagactgaaactcaaaggaattgacgggggcccgcacaagcagtggagtatgtggtttaattcgaagcaacgcgaagaaccttaccaggtcttgacatcgtgcgcataccgtagagatacgggaagtccttcgggacgcatagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcacttaacagagggaagcaagaccgcgaggtggagcaaacccccaaaaagtgtctcagttcggattgcaggctgcaacccgcctgtatgaagtcggaattgctagtaatcgcggatcagcatgccgcggtgaatacgttcccgggccttgtacacaccgcccgtcacaccatgagagccggtaacacccgaagtcagtagcctaaccgcaaggagggcgctgccgaaggtgggattggtgattagggtgaagtcgtaacaaggtagccgtatcggaaggtgcggctggatcacctccttt

SEQ ID NO: 4 - DNA, 1527 bp, Harryflintia acetispora strain A
tttagagagtttgatcctggctcaggatgaacgctggcggcgtgcctaacacatgcaagtcgaacggagttattttggaaatctcttcggagatggaatctttaacttagtggcggacgggtgagtaacgcgtgagcaatctgcctttaagagggggataacagtcggaaacggctgctaataccgcataaagcattgaattcgcatgttttcgatgccaaaggagcaatccgcttttagatgagctcgcgtctgattagctagttggcggggtaacggcccaccaaggcgacgatcagtagccggactgagaggttgaacggccacattgggactgagacacggcccagactcctacgggaggcagcagtggggaatattgcgcaatgggggaaaccctgacgcagcaacgccgcgtgattgaagaaggccttcgggttgtaaagatctttaattcgggacgaaaaatgacggtaccgaaagaataagctccggctaactacgtgccagcagccgcggtaatacgtagggagcaagcgttatccggatttactgggtgtaaagggcgcgcaggcgggctggcaagttggaagtgaaatctaggggcttaacccctaaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggatactaggtgtgggaggtattgaccccttccgtgccgcagttaacacaataagtatcccacctggggagtacggccgcaaggttgaaactcaaaggaattgacgggggcccgcacaagcagtggagtatgtggtttaattcgaagcaacgcgaagaaccttaccaggccttgacatcccgatgaccggtctagagatagaccttctcttcggagcatcggtgacaggtggtgcatggttgtcgtcagctcgtgtcgtgagatgttgggttaagtcccgcaacgagcgcaacccttacggttagttgatacgcaagatcactctagccggactgccgttgacaaaacggaggaaggtggggacgacgtcaaatcatcatgccccttatggcctgggctacacacgtactacaatggcagtcatacagagggaagcaaagctgtgaggcggagcaaatccctaaaagctgtcccagttcagattgcaggctgcaacccgcctgcatgaagtcggaattgctagtaatcgcggatcagcatgccgcggtgaatacgttcccgggccttgtacacaccgcccgtcacaccatgagagccgtcaatacccgaagtccgtagcctaaccgcaaggagggcgcggccgaaggtaggggtggtaattagggtgaagtcgtaacaaggtagccgtatcggaaggtgcggctggatcacctccttt

SEQ ID NO: 5 - DNA, 1532 bp, Acutalibacter sp. Strain A
tttagagagtttgatcctggctcaggacgaacgctggcggcgtgcctaacacatgcaagtcgaacggagataagcgctgatgatttagcttgctattgattcttgtttatcttagtggcggacgggtgagtaacgcgtgagcaacctgcctttcagagggggataacgtcttgaaaaggacgctaataccgcatgagatcgtagccccacatgggacagcgaccaaaggagcaatccgctgaaagatgggctcgcgtccgattagatagttggcggggtaacggcccaccaagtcgacgatcggtagccggactgagaggttgaacggccacattgggactgagacacggcccagactcctacgggaggcagcagtgagggatattggtcaatgggggaaaccctgaaccagcaacgccgcgtgagggatgacggccttcgggttgtaaacctctgtcctctgtgaagataatgacggtagcagaggaggaagctccggctaactacgtgccagcagccgcggtaatacgtagggagcaagcgttgtccggatttactgggtgtaaagggtgcgtaggcggttcggcaagtcagaagtgaaatccatgggcttaacccatgaactgcttttgaaactgtcgaacttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggtctgaccccctccgtgccggagttaacacaataagtaatccacctggggagtacggccgcaaggctgaaactcaaaggaattgacgggggcccgcacaagcagtggagtatgtggattaattcgaagcaacgcgaagaaccttaccaggtcttgacatccagctaacgaagtagagatacattaggtgcccttcggggaaagctgagacaggtggtgcatggttgtcgtcagctcgtgtcgtgagatgttgggttaagtcccgcaacgagcgcaacccttactgttagttgctacgcaagagcactctagcaggaccgccgttgacaaaacggaggaaggtggggatgatgtcaaatcatcatgccccttatgacctgggcctcacacgtactacaatggccgtaaacagagggaagcaataccgcgaggtggagcaaaaccctaaaaacggtcccagttcggattgtaggctgcaacccgcctgcatgaagttggaattgctagtaatcgcggatcagcatgccgcggtgaatacgttcccgggccttgtacacaccgcccgtcacaccatgggagccggtaatacccgaagtcagtagtctaaccgcaaggagggcgctgccgaaggtaggattggcgactggggtgaagtcgtaacaaggtagccgtatcggaaggtgcggctggatcacctccttt

SEQ ID NO: 6 - DNA, 1521 bp, Anaerotruncus colihominis Strain A
caaagagtttgatcctggctcaggacgaacgctggcggcgcgcctaacacatgcaagtcgaacggagcttagattttgaagttttcggatggatgaatgtaagcttagtggcggacgggtgagtaacacgtgagcaacctgcctttcagagggggataacagccggaaacggctgctaataccgcatgatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcatagcctagagataggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactaaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggggggcgctgtcgaaggtgggattgatgactggggtgaagtcgtaacaaggtagccgtatcggaaggtgcggctggatcacctccttt

SEQ ID NO: 7 - DNA, 523 bp, Subdoligranulum variabile Strain A
agatgcatgaagtccttcgggacatcgagacaggtggtgcatggttgtcgtcagctcgtgtcgtgagatgttgggttaagtcccgcaacgagcgcaacccttattgccagttactacgcaagaggactctggcgagactgccgttgacaaaacggaggaaggtggggatgacgtcaaatcatcatgccctttatgacctgggctacacacgtactacaatggcgtttaacaaagagaagcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtagccgtatcggaaggtgcggctggatcacctccttt

(19) United States
(12) Patent Application Publication
(10) Pub. No.: US 2023/0263838 A1
(43) Pub. Date: Aug. 24, 2023

(54) COMPOSITIONS AND METHODS FOR TREATING DISEASES AND DISORDERS USING OSCILLOSPIRACEAE MICROBIAL EXTRACELLULAR VESICLES

(71) Applicant: Evelo Biosciences, Inc., Cambridge, MA (US)

(72) Inventors: Alicia Ballok, Natick, MA (US); Loise Francisco-Anderson, Belmont, MA (US); Maria Sizova, Roslindale, MA (US)

(21) Appl. No.: 18/009,553

(22) PCT Filed: Jun. 11, 2021
(86) PCT No.: PCT/US21/36978

§ 371 Date: Dec. 9, 2022

Related U.S. Application Data
(60) Provisional application No. 63/037,771, filed on Jun. 11, 2020.

(51) Int. Cl. A61K 35/74; A61K 45/06; A61K 9/00; A61P 35/00; A61K 9/50

(52) U.S. Cl. CPC A61K 35/74; A61K 45/06; A61K 9/0053; A61P 35/00; A61K 9/5068

(57) ABSTRACT

Provided herein are methods and pharmaceutical compositions related to microbial extracellular vesicles (mEVs) obtained from Oscillospiraceae bacteria that can be useful as therapeutic agents.

### CROSS-REFERENCE TO RELATED APPLICATION

This application claims the benefit of U.S. Provisional Application No. 63/037,771, filed Jun. 11, 2020, the entire contents of which are incorporated herein by reference.

### SUMMARY

Provided herein are methods and compositions related to the use of Oscillospiraceae bacteria and/or microbial extracellular vesicles (mEVs) from Oscillospiraceae bacteria in the treatment and/or prevention of a disease or a health disorder (e.g., a cancer, an autoimmune disease, an inflammatory disease, a dysbiosis, and/or a metapbolic disease). As disclosed herein, certain types of mEVs such as secreted microbial extracellular vesicles (smEVs) or processed microbial extracellular vesicles (pmEVs) obtained from Oscillospiraceae bacteria have therapeutic effects and are useful for the treatment and/or prevention of a disease or a health disorder (e.g., a cancer, an autoimmune disease, an inflammatory disease, a dysbiosis, and/or a metabolic disease).

In some embodiments, a pharmaceutical composition provided herein contains mEVs (e.g., smEVs and/or pmEVs) from one or more Oscillospiraceae source, e.g., one or more Oscillospiraceae strain. In some embodiments, the pharmaceutical composition provided herein contains mEVs from one Oscillospiraceae source, e.g., one Oscillospiraceae strain. The Oscillospiraceae strain used as a source of mEVs may be selected based on the properties of the bacteria (e.g., growth characteristics, yield, ability to modulate an immune response in an assay or a subject). A pharmaceutical composition comprising mEVs can contain smEVs, pmEVs or a combination of both. In some embodiments, Oscillospiraceae strain is Faecalibacterium prausnitrzii (e.g., Faecalibacterium prausnitzii strain A), Fournierella massiliensis (e.g., Fournierella massiliensis strain A), Harryflintia acetispora (e.g., Harryflintia acetispora strain A), Agathobaculum sp. (e.g., Agathobaculum sp. strain A), Acutalibacter sp. (e.g., Acutalibacter sp. strain A), Anaerotruncus colihominis (Anaerotruncus colihominis strain A), or Subdoligranulum variabile (e.g., Subdoligranulum variabile strain A).

In certain aspects, provided herein is a pharmaceutical composition comprising mEVs (e.g., smEVs and/or pmEVs) from a strain of a Faecalibacterium prausnitzii. In some embodiments, the Faecalibacterium prausnitzii strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence. CRISPR sequence) of the Faecalibacterium prausnitzii Strain A (ATCC Deposit Number PTA-126792). In some embodiments, the Faecalibacterium prausnitzii strain is the Faecalibacterium prausnitzii Strain A (ATCC Deposit Number PTA-126792).

In certain aspects, provided herein is a pharmaceutical composition comprising mEVs from a strain of a Fournierella massiliensis. In some embodiments, the Fournierella massiliensis strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Fournierella massiliensis Strain A (ATCC Deposit Number PTA-126696). In some embodiments, the Fournierella massiliensis strain is the Fournierella massiliensis Strain A (ATCC Deposit Number PTA-126696).

In certain aspects, provided herein is a pharmaceutical composition comprising mEVs from a strain of a Harryflintia acetispora. In some embodiments, the Harryflintia acetispora strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Harryflintia acetispora Strain A (ATCC Deposit Number PTA-126694). In some embodiments, the Harryflintia acetispora strain is the Harryflintia acetispora Strain A (ATCC Deposit Number PTA-126694).

In certain aspects, provided herein is a pharmaceutical composition comprising mEVs from a strain of a Agathobaculum sp. In some embodiments, the Agathobaculum sp. strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Agathobaculum sp. Strain A (ATCC Deposit Number PTA-125892). In some embodiments, the Agathobaculum sp. strain is the Agathobaculum sp. Strain A (ATCC Deposit Number PTA-125892).

In certain aspects, provided herein is a pharmaceutical composition comprising mEVs from a strain of a Acutalibacter sp. In some embodiments, the Acutalibacter sp. strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Acutalibacter sp. Strain A (ATCC Deposit Number PTA-127006). In some embodiments, the Acutalibacter sp. strain is the Acutalibacter sp. Strain A (ATCC Deposit Number PTA-127006).

In certain aspects, provided herein is a pharmaceutical composition comprising mEVs from a strain of a Anaerotruncus colihominis. In some embodiments, the Anaerotruncus colihominis strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence. CRISPR sequence) of the Anaerotruncus colihominis Strain A (ATCC Deposit Number PTA-127005). In some embodiments, the Anaerotruncus colihominis strain is the Anaerotruncus colihominis Strain A (ATCC Deposit Number PTA-127005).

In certain aspects, provided herein is a pharmaceutical composition comprising mEVs from a strain of a Subdoligranulum variabile. In some embodiments, the Subdoligranulum variabile strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Subdoligranulum variabile Strain A (ATCC Deposit Number PTA-127004). In some embodiments, the Subdoligranulum variabile strain is the Subdoligranulum variabile Strain A (ATCC Deposit Number PTA-127004).

In some embodiments, a pharmaceutical composition provided herein comprising mEVs (such as smEVs and/or pmEVs) can be used for the treatment and/or prevention of a disease or a health disorder (e.g., a cancer, an autoimmune disease, an inflammatory disease, a dysbiosis, and/or a metabolic disease).

In some embodiments, a pharmaceutical composition provided herein comprising mEVs (such as smEVs and/or pmEVs) can be prepared as powder (e.g., for resuspension) or as a solid dose form, such as a tablet, a minitablet, a capsule, a pill, or a powder; or a combination of these forms (e.g., minitablets comprised in a capsule).

In some embodiments, a pharmaceutical composition provided herein can comprise lyophilized mEVs (such as smEVs and/or pmEVs). The lyophilized mEVs (such as smEVs or pmEVs) can be formulated into a solid dose form, such as a tablet, a minitablet, a capsule, a pill, or a powder; or can be resuspended in a solution.

In some embodiments, a pharmaceutical composition provided herein can comprise gamma irradiated mEVs (such as smEVs and/or pmEVs). The gamma irradiated mEVs (such as smEVs or pmEVs) can be formulated into a solid dose form, such as a tablet, a minitablet, a capsule, a pill, or a powder; or can be resuspended in a solution.

In some embodiments, a pharmaceutical composition provided herein comprising mEVs (such as smEVs and/or pmEVs) can be orally administered. In some embodiments, a pharmaceutical composition provided herein comprising mEVs (such as smEVs and/or pmEVs) can be administered intravenously. In some embodiments, a pharmaceutical composition provided herein comprising mEVs (such as smEVs and/or pmEVs) can be administered intratumorally or subtumorally, e.g., to a subject who has a tumor. In some embodiments, a pharmaceutical composition provided herein comprising mEVs (such as smEVs and/or pmEVs) can be topically administered.

In certain aspects, provided herein are pharmaceutical compositions comprising mEVs (such as smEVs and/or pmEVs) useful for the treatment and/or prevention of a disease or a health disorder (e.g., a cancer, an autoimmune disease, an inflammatory disease, a dysbiosis, and/or a metabolic disease), as well as methods of making and/or identifying such mEVs, and methods of using such pharmaceutical compositions (e.g., for the treatment and/or prevention of a disease or a health disorder (e.g., a cancer, an autoimmune disease, an inflammatory disease, a dysbiosis, and/or a metabolic disease), either alone or in combination with other therapeutics). In some embodiments, the pharmaceutical compositions comprise both mEVs and whole microbes from Oscillospiraceae (e.g., live bacteria, killed bacteria, attenuated bacteria). In some embodiments, the pharmaceutical compositions comprise mEVs in the absence of Oscillospiraceae from which they were obtained (e.g., over about 95% (or over about 99%) of the Oscillospiraceae-sourced content of the pharmaceutical composition comprises mEVs).

In some embodiments, the pharmaceutical composition comprises isolated mEVs (e.g., from one or more strains of Oscillospiraceae) (e.g., a therapeutically effective amount thereof). E.g., wherein at least 50%, at least 75%, at least 80%, at least 85%, at least 90%, at least 95%, or at least 99% of the content of the pharmaceutical composition is isolated mEV of Oscillospiraceae. In some embodiments, Oscillospiraceae strain is Faecalibacterium prausnitzii (e.g., Faecalibacterium prausnitzii strain A), Fournierella massiliensis (e.g., Fournierella massiliensis strain A). Harryflintia acetispora (e.g., Harryflintia acetispora strain A), Agathobaculum sp. (e.g., Agathobaculum sp strain A), Acutalibacter sp. (e.g., Acutalibacter sp. strain A), Anaerotruncus colihominis (Anaerotruncus colihominis strain A), or Subdoligranulum variabile (e.g., Subdoligranulum variabile strain A).

In some embodiments, the pharmaceutical composition comprises isolated mEVs (e.g., from one strain of Oscillospiraceae) (e.g., a therapeutically effective amount thereof). E.g., wherein at least 50%, at least 75%, at least 80%, at least 85%, at least 90%, at least 95%, or at least 99% of the content of the pharmaceutical composition is isolated mEV of Oscillospiraceae. In some embodiments, Oscillospiraceae strain is Faecalibacterium prausnitzii (e.g., Faecalibacterium prausnitzii strain A), Fournierella massiliensis (e.g., Fournierella massiliensis strain A), Harryflintia acetispora (e.g., Harryflintia acetispora strain A). Agathobaculum sp. (e.g., Agathobaculum sp. strain A). Acutalibacter sp. (e.g., Acutalibacter sp. strain A), Anaerotruncus colihominis (Anaerotruncus colihominis strain A), or Subdoligranulum variabile (e.g., Subdoligranulum variabile strain A).

In some embodiments, the pharmaceutical composition comprises mEVs and the mEVs are produced from a high yield strain. In some embodiments, the high yield strain produces at least 3×10¹³ mEVs per liter from a bioreactor-grown culture.

In some embodiments, the pharmaceutical composition comprises secreted mEVs (smEVs).

In some embodiments, the pharmaceutical composition comprises smEVs and the smEVs are produced from live bacteria.

In some embodiments, the pharmaceutical composition comprises smEVs and the smEVs are produced from a high yield strain. In some embodiments, the high yield strain produces at least 3×10¹³ smEVs per liter from a bioreactor-grown culture.

In some embodiments, the pharmaceutical composition comprises smEVs and the smEVs are from one strain of Oscillospiraceae. In some embodiments, the pharmaceutical composition comprises mEVs and the mEVs are from one strain of Oscillospiraceae. In some embodiments, Oscillospiraceae strain is Faecalibacterium prausnitzii (e.g., Faecalibacterium prausnitzii strain A), Fournierella massiliensis (e.g., Fournierella massiliensis strain A). Harryflintia acetispora (e.g., Harryflintia acetispora strain A), Agathobaculum sp. (e.g., Agathobaculum sp. strain A), Acutalibacter sp. (e.g., Acutalibacter sp. strain A), Anaerotruncus colihominis (Anaerotruncus colihominis strain A), or Subdoligranulum variabile (e.g., Subdoligranulum variabile strain A).

In some embodiments, the pharmaceutical composition comprises processed mEVs (pmEVs).

In some embodiments, the pharmaceutical composition comprises pmEVs and the pmEVs are produced from bacteria that have been gamma irradiated, UV irradiated, heat inactivated, acid treated, or oxygen sparged.

In some embodiments, the pharmaceutical composition comprises pmEVs and the pmEVs are produced from live bacteria. In some embodiments, the pharmaceutical composition comprises pmEVs and the pmEVs are produced from dead bacteria. In some embodiments, the pharmaceutical composition comprises pmEVs and the pmEVs are produced from non-replicating bacteria.

In some embodiments, the pharmaceutical composition comprises pmEVs and the pmEVs are produced from a high yield strain. In some embodiments, the high yield strain produces at least 3×10¹³ pmEVs per liter from a bioreactor-grown culture.

In some embodiments, the pharmaceutical composition comprises pmEVs and the pmEVs are from one strain of Oscillospiraceae. In some embodiments, the pharmaceutical composition comprises mEVs and the mEVs are from one strain of Oscillospiraceae. In some embodiments, Oscillospiraceae strain is Faecalibacterium prausnitzii (e.g., Faecalibacterium prausnitzii strain A). Fournierella massiliensis (e.g., Fournierella massiliensis strain A), Harryflintia acetispora (e.g., Harryflintia acetispora strain A), Agathobaculum sp. (e.g., Agathobaculum sp. strain A). Acutalibacter sp. (e.g., Acutalibacter sp. strain A), Anaerotruncus colihominis (Anaerotruncus colihominis strain A), or Subdoligranulum variabile (e.g., Subdoligranulum variabile strain A).

In some embodiments, the mEVs (such as smEVs and/or pmEVs) are lyophilized (e.g., the lyophilized product further comprises a pharmaceutically acceptable excipient). In some embodiments, the mEVs are gamma irradiated. In some embodiments, the mEVs are UV irradiated. In some embodiments, the mEVs are heat inactivated (e.g., at 50° C. for two hours or at 90° C. for two hours). In some embodiments, the mEVs are acid treated. In some embodiments, the mEVs are oxygen sparged (e.g., at 0.1 vvm for two hours).

In certain aspects, the mEVs (such as smEVs and/or pmEVs) are obtained from Oscillospiraceae bacteria that have been selected based on certain desirable properties, such as reduced toxicity and adverse effects (e.g., by removing or deleting lipopolysaccharide (LPS)), enhanced oral delivery (e.g., by improving acid resistance, muco-adherence and/or penetration and/or resistance to bile acids, resistance to anti-microbial peptides and/or antibody neutralization), target desired cell types (e.g., M-cells, goblet cells, enterocytes, dendritic cells, macrophages), improved bioavailability systemically or in an appropriate niche (e.g., mesenteric lymph nodes, Peyer's patches, lamina propria, tumor draining lymph nodes, and/or blood), enhanced immunomodulatory and/or therapeutic effect (e.g., either alone or in combination with another therapeutic agent), enhanced immune activation, and/or manufacturing attributes (e.g., growth characteristics, yield, greater stability, improved freeze-thaw tolerance, shorter generation times).

In certain aspects, the mEVs (such as smEVs and/or pmEVs) are from engineered Oscillospiraceae bacteria that are modified to enhance certain desirable properties. In some embodiments, the engineered Oscillospiraceae bacteria are modified so that mEVs (such as smEVs and/or pmEVs) produced therefrom will have reduced toxicity and adverse effects (e.g., by removing or deleting lipopolysaccharide (LPS)), enhanced oral delivery (e.g., by improving acid resistance, muco-adherence and/or penetration and/or resistance to bile acids, resistance to anti-microbial peptides and/or antibody neutralization), target desired cell types (e.g., M-cells, goblet cells, enterocytes, dendritic cells, macrophages), improved bioavailability systemically or in an appropriate niche (e.g., mesenteric lymph nodes, Peyer's patches, lamina propria, tumor draining lymph nodes, and/or blood), enhanced immunomodulatory and/or therapeutic effect (e.g., either alone or in combination with another therapeutic agent), enhanced immune activation, and/or improved manufacturing attributes (e.g., growth characteristics, yield, greater stability, improved freeze-thaw tolerance, shorter generation times). In some embodiments, provided herein are methods of making such mEVs (such as smEVs and/or pmEVs).

In certain aspects, provided herein are pharmaceutical compositions comprising mEVs (such as smEVs and/or pmEVs) from Oscillospiraceae useful for the treatment and/or prevention of a disease or a health disorder (e.g., a cancer, an autoimmune disease, an inflammatory disease, a dysbiosis, and/or a metabolic disease), as well as methods of making and/or identifying such mEVs, and methods of using such pharmaceutical compositions (e.g., for the treatment and/or prevention of a disease or a health disorder (e.g., a cancer, an autoimmune disease, an inflammatory disease, a dysbiosis, and/or a metabolic disease)), either alone or in combination with one or more other therapeutics.

Pharmaceutical compositions containing mEVs (such as smEVs and/or pmEVs) from Oscillospiraceae can provide potency comparable to or greater than pharmaceutical compositions that contain the whole microbes from which the mEVs were obtained. For example, at the same dose of mEVs (e.g., based on particle count or protein content), a pharmaceutical composition containing mEVs can provide potency comparable to or greater than a comparable pharmaceutical composition that contains whole microbes of the same Oscillospiraceae strain from which the mEVs were obtained. Such mEV containing pharmaceutical compositions can allow the administration of higher doses and elicit a comparable or greater (e.g., more effective) response than observed with a comparable pharmaceutical composition that contains whole microbes of the same Oscillospiraceae strain from which the mEVs were obtained. In some embodiments, Oscillospiraceae strain is Faecalibacterium prausnitzii (e.g., Faecalibacterium prausnitzii strain A), Fournierella massiliensis (e.g., Fournierella massiliensis strain A), Harryflintia acetispora (e.g., Harryflintia acetispora strain A). Agathobaculum sp. (e.g., Agathobaculum sp. strain A). Acutalibacter sp. (e.g., Acutalibacter sp. strain A), Anaerotruncus colihominis (Anaerotruncus colihominis strain A), or Subdoligranulum variable (e.g., Subdoligranulum variabile strain A).

As a further example, at the same dose (e.g., based on particle count or protein content), a pharmaceutical composition containing mEVs may contain less microbially-derived material (based on particle count or protein content), as compared to a pharmaceutical composition that contains the whole microbes of the same Oscillospiraceae strain from which the mEVs were obtained, while providing an equivalent or greater therapeutic benefit to the subject receiving such pharmaceutical composition. In some embodiments, Oscillospiraceae strain is Faecalibacterium prausnitzii (e.g., Faecalibacterium prausnitzii strain A), Fournierella massiliensis (e.g., Fournierella massiliensis strain A), Harryflintia acetispora (e.g., Harryflintia acetispora strain A), Agathobaculum sp. (e.g., Agathobaculum sp. strain A), Acutalibacter sp. (e.g., Acutalibacter sp. strain A), Anaerotruncus colihominis (Anaerotruncus colihominis strain A), or Subdoligranulum variabile (e.g., Subdoligranulum variabile strain A).

As a further example, mEVs can be administered at doses e.g., of about 1×10⁷-about 1×10′⁵ particles, e.g., as measured by NIA. As another example, mEVs can be administered at doses e.g., of about 5 mg to about 900 mg total protein, e.g., as measured by Bradford assay or BCA assay.

In certain embodiments, provided herein are methods of treating a subject who has cancer comprising administering to the subject a pharmaceutical composition described herein. In some embodiments, a use of at least one pharmaceutical composition described herein is for treating or preventing cancer in a subject. In certain embodiments, provided herein are methods of treating a subject who has dysbiosis comprising administering to the subject a pharmaceutical composition described herein. In some embodiments, a use of at least one pharmaceutical composition described herein is for treating or preventing dysbiosis in a subject.

In certain embodiments, provided herein are methods of treating a subject who has an immune disorder (e.g., an autoimmune disease, an inflammatory disease, an allergy) comprising administering to the subject a pharmaceutical composition described herein. In some embodiments, a use of at least one pharmaceutical composition described herein is for treating or preventing immune disorder in a subject.

In certain embodiments, provided herein are methods of treating a subject who has a metabolic disease comprising administering to the subject a pharmaceutical composition described herein. In some embodiments, a use of at least one pharmaceutical composition described herein is for treating or preventing a metabolic disease in a subject.

In certain embodiments, provided herein are methods of treating a subject who has a neurologic disease comprising administering to the subject a pharmaceutical composition described herein. In some embodiments, a use of at least one pharmaceutical composition described herein is for treating or preventing a neurologic disease in a subject.

In some embodiments, the pharmaceutical composition described herein is administered once a day. In some embodiments, the pharmaceutical composition described herein is administered twice a day. In some embodiments, the pharmaceutical composition described herein is formulated for a daily dose. In some embodiments, the pharmaceutical composition described herein is formulated for twice a day dose, wherein each dose is half of the daily dose.

In some embodiments, the method further comprises administering to the subject an antibiotic.

In some embodiments, the method further comprises administering to the subject one or more cancer therapies (e.g., surgical removal of a tumor, the administration of a chemotherapeutic agent, the administration of radiation therapy, and/or the administration of a cancer immunotherapy, such as an immune checkpoint inhibitor, a cancer-specific antibody, a cancer vaccine, a primed antigen presenting cell, a cancer-specific T cell, a cancer-specific chimeric antigen receptor (CAR) T cell, an immune activating protein, and/or an adjuvant). In some embodiments, the method further comprises the administration of another therapeutic bacterium and/or mEVs (such as smEVs and/or pmEVs) from one or more other bacterial strains (e.g., therapeutic bacterium).

In some embodiments, the method further comprises the administration of an immune suppressant and/or an anti-inflammatory agent. In some embodiments, the method further comprises the administration of a metabolic disease therapeutic agent.

In certain aspects, provided herein is a pharmaceutical composition comprising mEVs (such as smEVs and/or pmEVs) from Oscillospiraceae for use in the treatment and/or prevention of a disease or a health disorder (e.g., a cancer, an autoimmune disease, an inflammatory disease, a dysbiosis, and/or a metabolic disease), either alone or in combination with one or more other therapeutic agent.

In certain embodiments, provided herein is a pharmaceutical composition comprising mEVs (such as smEVs and/or pmEVs) from Oscillospiraceae for use in treating and/or preventing a cancer in a subject (e.g., human) The pharmaceutical composition can be used either alone or in combination with one or more other therapeutic agent for the treatment of the cancer. In certain embodiments, provided herein is a pharmaceutical composition comprising mEVs (such as smEVs and/or pmEVs) from Oscillospiraceae for use in treating and/or preventing a dysbiosis in a subject (e.g., human). The pharmaceutical composition can be used either alone or in combination with therapeutic agent for the treatment of the dysbiosis. In certain embodiments, provided herein is a pharmaceutical composition comprising mEVs (such as smEVs and/or pmEVs) for use m treating and/or preventing a metabolic disease in a subject (e.g., human). The pharmaceutical composition can be used either alone or in combination with therapeutic agent for the treatment of the metabolic disease. In certain embodiments, provided herein is a pharmaceutical composition comprising mEVs (such as smEVs and/or pmEVs) for use in treating and/or preventing a neurologic disease in a subject (e.g., human). The pharmaceutical composition can be used either alone or in combination with one or more other therapeutic agent for treatment of the neurologic disorder.

In some embodiments, the pharmaceutical composition comprising mEVs can be for use in combination with an antibiotic. In some embodiments, the pharmaceutical composition comprising mEVs can be for use in combination with one or more other cancer therapies (e.g., surgical removal of a tumor, the use of a chemotherapeutic agent, the use of radiation therapy, and/or the use of a cancer immunotherapy, such as an immune checkpoint inhibitor, a cancer-specific antibody, a cancer vaccine, a primed antigen presenting cell, a cancer-specific T cell, a cancer-specific chimeric antigen receptor (CAR) T cell, an immune activating protein, and/or an adjuvant). In some embodiments, the pharmaceutical composition comprising mEVs can be for use in combination with another therapeutic bacterium and/or mEVs obtained from one or more other Oscillospiraceae strains. In some embodiments, Oscillospiraceae strain is Faecalibacterium prausnitzii (e.g., Faecalibacterium prausnitzii strain A). Fournierella massiliensis (e.g., Fournierella massiliensis strain A). Harryflintia acetispora (e.g., Harryflintia acetispora strain A), Agathobaculum sp. (e.g., Agathobaculum sp. strain A), Acutalibacter sp (e.g., Acutalibacter sp. strain A), Anaerotruncus colihominis (Anaerotruncus colihominis strain A), or Subdoligranulum variabile (e.g., Subdoligranulum variabile strain A).

In some embodiments, the pharmaceutical composition comprising mEVs can be for use in combination with one or more immune suppressant(s) and/or an anti-inflammatory agent(s). In some embodiments, the pharmaceutical composition comprising mEVs can be for use in combination with one or more other metabolic disease therapeutic agents.

In certain aspects, provided herein is use of a pharmaceutical composition comprising mEVs (such as smEVs and/or pmEVs) from Oscillospiraceae for the preparation of a medicament for the treatment and/or prevention of a disease or a health disorder (e.g., a cancer, an autoimmune disease, an inflammatory disease, a dysbiosis, and/or a metabolic disease), either alone or in combination with another therapeutic agent. In some embodiments, the use is in combination with another therapeutic bacterium and/or mEVs obtained from one or more other Oscillospiraceae strains. In some embodiments, Oscillospiraceae strain is Faecalibacterium prausnitzii (e.g., Faecalibacterium prausnitzii strain A), Fournierella massiliensis (e.g., Fournierella massiliensis strain A), Harryflintia acetispora (e.g., Harryflintia acetispora strain A), Agathobaculum sp. (e.g., Agathobaculum sp strain A), Acutalibacter sp. (e.g., Acutalibacter sp. strain A), Anaerotruncus colihominis (Anaerotruncus colihominis strain A), or Subdoligranulum variabile (e.g., Subdoligranulum variabile strain A).

In certain embodiments, provided herein is use of a pharmaceutical composition comprising mEVs (such as smEVs and/or pmEVs) from Oscillospiraceae for the preparation of a medicament for treating and/or preventing a cancer in a subject (e.g., human). The pharmaceutical composition can be for use either alone or in combination with another therapeutic agent for the cancer. In certain embodiments, provided herein is use of a pharmaceutical composition comprising mEVs (such as smEVs and/or pmEVs) from Oscillospiraceae for the preparation of a medicament for treating and/or preventing a dysbiosis in a subject (e.g., human). The pharmaceutical composition can be for use either alone or in combination with another therapeutic agent for the dysbiosis.

In certain embodiments, provided herein is use of a pharmaceutical composition comprising mEVs (such as smEVs and/or pmEVs) for the preparation of a medicament for treating and/or preventing a metabolic disease in a subject (e.g., human). The pharmaceutical composition can be for use either alone or in combination with another therapeutic agent for the metabolic disease. In certain embodiments, provided herein is use of a pharmaceutical composition comprising mEVs (such as smEVs and/or pmEVs) for the preparation of a medicament for treating and or preventing a neurologic disease in a subject (e.g., human) The pharmaceutical composition can be for use either alone or in combination with another therapeutic agent for the neurologic disorder.

In some embodiments, the pharmaceutical composition comprising mEVs can be for use in combination with an antibiotic. In some embodiments, the pharmaceutical composition comprising mEVs can be for use in combination with one or more other cancer therapies (e.g., surgical removal of a tumor, the use of a chemotherapeutic agent, the use of radiation therapy, and/or the use of a cancer immunotherapy, such as an immune checkpoint inhibitor, a cancer-specific antibody, a cancer vaccine, a primed antigen presenting cell, a cancer-specific T cell, a cancer-specific chimeric antigen receptor (CAR) T cell, an immune activating protein, and/or an adjuvant). In some embodiments, the pharmaceutical composition comprising mEVs can be for use in combination with another therapeutic bacterium and/or mEVs obtained from one or more other Oscillospiraceae strains. In some embodiments, Oscillospiraceae strain is Faecalibacterium prausnitzii (e.g., Faecalibacterium prausnitrzii strain A), Fournierella massiliensis (e.g., Fournierella massiliensis strain A), Harryflintia acetispora (e.g., Harryflintia acetispora strain A), Agathobaculum sp. (e.g., Agathobaculum sp. strain A), Acutalibacter sp. (e.g., Acutalibacter sp. strain A), Anaerotruncus colihominis (Anaerotruncus colihominis strain A), or Subdoligranulum variabile (e.g., Subdoligranulum variabile strain A).

In some embodiments, the pharmaceutical composition comprising mEVs can be for use in combination with one or more other immune suppressant(s) and/or an anti-inflammatory agent(s). In some embodiments, the pharmaceutical composition can be for use in combination with one or more other metabolic disease therapeutic agent(s).

A pharmaceutical composition, e.g., as described herein, comprising mEVs (such as smEVs and/or pmEVs) from Oscillospiraceae can provide a therapeutically effective amount of mEVs to a subject, e.g., a human.

A pharmaceutical composition, e.g., as described herein, comprising mEVs (such as smEVs and/or pmEVs) from Oscillospiraceae can provide a non-natural amount of the therapeutically effective components (e.g., present in the mEVs (such as smEVs and/or pmEVs)) to a subject, e.g., a human.

A pharmaceutical composition, e.g., as described herein, comprising mEVs (such as smEVs and/or pmEVs) from Oscillospiraceae can provide unnatural quantity of the therapeutically effective components (e.g., present in the mEVs (such as smEVs and/or pmEVs)) to a subject. e.g., a human.

A pharmaceutical composition, e.g., as described herein, comprising mEVs (such as smEVs and/or pmEVs) from Oscillospiraceae can bring about one or more changes to a subject. e.g., human, e.g., to treat or prevent a disease or a health disorder.

A pharmaceutical composition, e.g., as described herein, comprising mEVs (such as smEVs and/or pmEVs) from Oscillospiraceae has potential for significant utility, e.g., to affect a subject. e.g., a human, e.g., to treat or prevent a disease or a health disorder.

In certain aspects, mEVs (such as smEVs and/or pmEVs) from Oscillospiraceae, or any combination thereof and/or a pharmaceutical composition comprising the mEVs (such as smEVs and/or pmEVs) reduce tumor growth m a CT26 preclinical model of cancer.

In certain aspects, mEVs (such as smEVs and/or pmEVs) from Oscillospiraceae, or any combination thereof and/or a pharmaceutical composition comprising the mEVs (such as smEVs and/or pmEVs) reduce ear thickness in a DTH (delayed type hypersensitivity) preclinical model of inflammation.

In certain aspects, mEVs (such as smEVs and/or pmEVs) from Oscillospiraceae, or any combination thereof and/or a pharmaceutical composition comprising the mEVs (such as smEVs and/or pmEVs) induce cytokine production from PMA-differentiated U937 cells. In some embodiments, the mEVs (such as smEVs and/or pmEVs) from Oscillospiraceae, or any combination thereof and/or a pharmaceutical composition comprising the mEVs (such as smEVs and/or pmEVs) induce production of one or more of IL-10; TNF-α; IL-6; IP-10; and IL-1β (e.g., as compared to a blank control) In certain aspects, mEVs (such as smEVs and/or pmEVs) from Oscillospiraceae, or any combination thereof and/or a pharmaceutical composition comprising the mEVs (such as smEVs and/or pmEVs) activate TLR signaling, e.g., in vivo or in a reporter cell assay (e.g., HEK293-SEAP reporter cell assay). In some embodiments, the mEVs (such as smEVs and/or pmEVs) from Oscillospiraceae, or any combination thereof and/or a pharmaceutical composition comprising the mEVs (such as smEVs and/or pmEVs) activate one or more of: TLR2 and TLR5 signaling.

### BRIEF DESCRIPTION OF THE FIGURES

FIG. 1 shows the efficacy of smEVs from F. massiliensis Strain A as compared to that of intraperitoneally (i.p.) administered anti-PD-1 or vehicle in a mouse colorectal carcinoma model at day 11. Welch's test is performed for treatment vs. vehicle.

FIG. 2 shows the efficacy of orally administered smEVs from F. massiliensis Strain A as compared to that of intraperitoneally (i.p.) administered anti-PD-1 or vehicle in a mouse colorectal carcinoma model over a period of time. Welch's test is performed for treatment vs. vehicle.

FIG. 3 shows the efficacy of orally administered smEVs from F. massiliensis Strain A as compared to that of intraperitoneally (i.p.) administered anti-PD-1 or vehicle in a mouse colorectal carcinoma model at day 23. Welch's test is performed for treatment vs. vehicle.

FIG. 4 shows the efficacy of orally administered smEVs from F. massiliensis Strain A as compared to that of intraperitoneally (i.p.) administered anti-PD-1 or vehicle in a mouse colorectal carcinoma model over a period of time. Welch's test is performed for treatment vs. vehicle.

FIG. 5 shows the efficacy of orally administered smEVs from F. massiliensis Strain A as compared to that of intraperitoneally (i.p.) administered anti-PD-1 or vehicle in a mouse colorectal carcinoma model at day 21. Welch's test is performed for treatment vs. vehicle.

FIG. 6 shows the efficacy of orally administered smEVs from F. massiliensis Strain A as compared to that of intraperitoneally (i.p.) administered anti-PD-1 or vehicle in a mouse colorectal carcinoma model over a period of time. Welch's test is performed for treatment vs. vehicle.

FIG. 7 shows the efficacy of orally administered smEVs from H. acetispora Strain A as compared to that of intraperitoneally (i.p.) administered anti-PD-1 or vehicle in a mouse colorectal carcinoma model at day 22. Welch's test is performed for treatment vs. vehicle.

FIG. 8 shows the efficacy of orally administered smEVs from H. acetispora Strain A as compared to that of intraperitoneally (i.p.) administered anti-PD-1 or vehicle in a mouse colorectal carcinoma model over a period of time. Welch's test is performed for treatment vs. vehicle.

FIG. 9 shows the efficacy of smEVs from H. acetispora Strain A as compared to that of intraperitoneally (i.p.) administered anti-PD-1 or vehicle in a mouse colorectal carcinoma model at day 23. Welch's test is performed for treatment vs. vehicle.

FIG. 10 shows the efficacy of orally administered smEVs from H. acetispora Strain A as compared to that of intraperitoneally (i.p.) administered anti-PD-1 or vehicle in a mouse colorectal carcinoma model over a period of time. Welch's test is performed for treatment vs. vehicle.

FIG. 11 shows the efficacy of orally administered smEVs from H. acetispora Strain A as compared to that of intraperitoneally (i.p.) administered anti-PD-1 or vehicle in a mouse colorectal carcinoma model at day 21. Welch's test is performed for treatment vs. vehicle.

FIG. 12 shows the efficacy of orally administered smEVs from H. acetispora Strain A as compared to that of intraperitoneally (i.p.) administered anti-PD-1 or vehicle in a mouse colorectal carcinoma model over a period of time. Welch's test is performed for treatment vs. vehicle.

FIG. 13 shows the efficacy of orally administered smEVs from Fournierella massillensis Strain A at two doses, 2E+11 and 2E+07 (based on particles per dose) in reducing antigen-specific ear swelling (ear thickness) at 24 hours compared to vehicle (negative control) and dexamethasone (positive control) following antigen challenge in a KLH-based delayed type hypersensitivity model.

FIG. 14 shows the efficacy of orally administered smEVs from Harryflintia acetispora Strain A at two doses, 2E+11 and 2E+07 (based on particles per dose) in reducing antigen-specific ear swelling (ear thickness) at 24 hours compared to vehicle (negative control) and dexamethasone (positive control) following antigen challenge in a KLH-based delayed type hypersensitivity model.

FIG. 15 shows the efficacy of orally administered smEVs from Harryflintia acetispora Strain A at two doses, 2E+11, 2E+09, and 2E+07 (based on particles per dose) in reducing antigen-specific ear swelling (ear thickness) at 24 hours compared to vehicle (negative control) and dexamethasone (positive control) following antigen challenge in a KLH-based delayed type hypersensitivity model.

FIG. 16 shows the efficacy of orally administered smEVs from Faecalibacterium prausnitzii Strain A at two doses, 2E+11 and 2E+07 (based on particles per dose) in reducing antigen-specific ear swelling (car thickness) at 24 hours compared to vehicle (negative control) and dexamethasone (positive control) following antigen challenge in a KLH-based delayed type hypersensitivity model.

FIG. 17 shows the efficacy of orally administered smEVs from Faecalibacterium prausnitzii Strain A at three doses, 2E+11, 2E+09, and 2E+07 (based on particles per dose) in reducing antigen-specific ear swelling (ear thickness) at 24 hours compared to vehicle (negative control) and dexamethasone (positive control) following antigen challenge in a KLH-based delayed type hypersensitivity model.

FIG. 18 shows smEVs from Fournierella massiliensis Strain A induce cytokine production from PMA-differentiated U937 cells. U937 cells were treated with Fournierella massiliensis Strain A smEV at 1×10⁶-1×10⁹ concentrations as well as TLR2 (FSL) and TLR4 (LPS) agonist controls for 24 hrs and cytokine production was measured. Note the strong effect seen at low concentrations of smEVs from this strain. “Blank” indicates the medium control.

FIG. 19 shows smEVs from Harryflintia acetispora Strain A induce cytokine production from PMA-differentiated U937 cells. U937 cells were treated with Harryflintia acetispora Strain A smEV at 1×10⁶-1×10⁹ concentrations as well as TLR2 (FSL) and TLR4 (LPS) agonist controls for 24 hrs and cytokine production was measured. Note the stepwise increase in cytokine production. “Blank” indicates the medium control.

FIG. 20 shows smEVs from Faecalibacterium prausnitzii Strain A induce cytokine production from PMA-differentiated U937 cells. U937 cells were treated with Faecalibacterium prausnitzii Strain A smEV at 1×10⁶-1×10⁹ concentrations as well as TLR2 (FSL) and TLR4 (LPS) agonist controls for 24 hrs and cytokine production was measured. Note the strong effect seen at low concentrations of smEVs from this strain. “Blank” indicates the medium control.

FIG. 21 shows smEVs from Acutalibacter sp. Strain A, Anaerotruncus colihominis Strain A, and Subdoligranulum variabile Strain A induce cytokine production from PMA-differentiated U937 cells. U937 cells were treated with smEV at 1×10⁶-1×10⁹ concentrations as well as TLR2 (FSL) and TLR4 (LPS) agonist controls for 24 hrs and cytokine production was measured. “Blank” indicates the medium control.

FIG. 22 shows the efficacy of orally administered smEVs from Acutalibacter sp. Strain A, Anaerotruncus colihominis Strain A, and Subdoligranulum variabile Strain A at two doses, 2E+10 and 2E+06 (based on particles per dose) in reducing antigen-specific ear swelling (ear thickness) at 24 hours compared to vehicle (negative control) and dexamethasone (positive control) following antigen challenge in a KLH-based delayed type hypersensitivity model.

FIG. 23 shows the effects of two batches of orally administered smEVs from Subdoligranulum variabile Strain A at 2E+10 (based on particles per dose) in reducing antigen-specific ear swelling (car thickness) at 24 hours compared to vehicle (negative control) and dexamethasone (positive control) following antigen challenge in a KLH-based delayed type hypersensitivity model.

FIG. 24 shows the efficacy of orally administered smEVs from Anaerotruncus colihominis Strain A at two doses, 2E+10 and 2E+06 (based on particles per dose) in reducing antigen-specific ear swelling (ear thickness) at 24 hours compared to vehicle (negative control) and dexamethasone (positive control) following antigen challenge in a KLH-based delayed type hypersensitivity model.

FIG. 25 shows the efficacy of three batches of Anaerotruncus colihominis strain A smEVs on various TLR receptors in HEK293-SEAP reporter cells and using absorbance at OD 630 nm as the read-out.

### DETAILED DESCRIPTION

The Oscillospiraceae family within the Clostridia class of microorganisms are common commensal organisms of vertebrates. Interestingly, despite being modern (usually described as Gram-positive) microbes they stain Gram-negative. We investigated whether members of this family could produce smEVs in sufficient yield to use commercially and found that relative to other Gram-positive organisms, members of this family produced high levels of smEVs. This defies the conventional understanding of smEV production within the field, as Gram-negative organisms typically produce higher numbers of smEVs. Oscillospiraceae family microbes are generally difficult to both isolate and grow, thus previous investigations of smEVs from this family have been limited. From our investigations each Oscillospiraceae smEV clicits a unique cytokine response in in vitro coculture. smEVs from Fournierella massiliensis [e.g., Fournierella massiliensis Strain A] and Harryflintia acetispora [e.g., Harryflintia acetispora Strain A] have demonstrated high efficacy and potency in animal models of inflammation and cancer. Faecalibacterium prausnitzii [e.g., Faecalibacterium prausnitzii Strain A] smEVs have shown therapeutic efficacy as well. Provided herein are methods and compositions related to the use of Oscillospiraceae bacteria in the treatment and/or prevention of a disease or a health disorder (e.g., a cancer, an autoimmune disease, an inflammatory disease, a dysbiosis, and/or a metabolic disease).

High Yield Criterion for smEV Production: A crude smEV yield of 3×10¹³ per liter from a bioreactor-grown culture is the minimum threshold of the “high yield” designation for strains because this number was within one order of magnitude of the crude smEV yield of a reference strain that produces smEVs at a level that can reasonably be scaled for production.

Table 1 lists smEV yields from different Oscillospiraceae strains.

| | | | EV |
|---|---|---|---|
| Strain | Classification | Strain Name | Yield/Liter |
| Strain A | Oscillospiraceae | Faecalibacterium prausnitzii | 1.30E+14 |
| | | S31RS2-T1-2 | |
| Strain A | Oscillospiraceae | Fournierella massiliensis | 8.80E+13 |
| | | S10 GIMucosa-297 | |
| Strain A | Oscillospiraceae | Harryflintia acetispora S4-M5 | 3.20E+13 |
| Strain A | Oscillospiraceae | Agathobaculum sp. | 1.32E+12 |

### Definitions

“Adjuvant” or “Adjuvant therapy” broadly refers to an agent that affects an immunological or physiological response in a patient or subject (e.g., human). For example, an adjuvant might increase the presence of an antigen over time or to an area of interest like a tumor, help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition (e.g., a pharmaceutical composition) to a subject. Examples of routes of administration include oral administration, rectal administration, topical administration, inhalation (nasal) or injection. Administration by injection includes intravenous (IV), intramuscular (IM), intratumoral (IT) and subcutaneous (SC) administration. A pharmaceutical composition described herein can be administered in any form by any effective route, including but not limited to intratumoral, oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In preferred embodiments, a pharmaceutical composition described herein is administered orally, rectally, intratumorally, topically, intravesically, by injection into or adjacent to a draining lymph node, intravenously, by inhalation or aerosol, or subcutaneously. In another preferred embodiment, a pharmaceutical composition described herein is administered orally, intratumorally, or intravenously.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refer to one or more fragments of an antibody that retain the ability to bind to an antigen. Examples of binding fragments encompassed within the term “antigen-binding fragment” of an antibody include Fab. Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

“Cancer” broadly refers to an uncontrolled, abnormal growth of a host's own cells leading to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue “Cancer(s) and” “neoplasm(s)”” are used herein interchangeably. As used herein, “cancer” refers to all types of cancer or neoplasm or malignant tumors including leukemias, carcinomas and sarcomas, whether new or recurring. Specific examples of cancers are: carcinomas, sarcomas, myelomas, leukemias, lymphomas and mixed type tumors. Non-limiting examples of cancers are new or recurring cancers of the brain, melanoma, bladder, breast, cervix, colon, head and neck, kidney, lung, non-small cell lung, mesothelioma, ovary, prostate, sarcoma, stomach, uterus and medulloblastoma. In some embodiments, the cancer comprises a solid tumor. In some embodiments, the cancer comprises a metastasis.

A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

“Cellular augmentation” broadly refers to the influx of cells or expansion of cells in an environment that are not substantially present in the environment prior to administration of a composition and not present in the composition itself. Cells that augment the environment include immune cells, stromal cells, bacterial and fungal cells. Environments of particular interest are the microenvironments where cancer cells reside or locate. In some instances, the microenvironment is a tumor microenvironment or a tumor draining lymph node. In other instances, the microenvironment is a pre-cancerous tissue site or the site of local administration of a composition or a site where the composition will accumulate after remote administration.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

A “combination” of mEVs (such as smEVs and/or pmEVs) from two or more microbial strains includes the physical co-existence of the microbes from which the mEVs (such as smEVs and/or pmEVs) are obtained, either in the same material or product or in physically connected products, as well as the temporal co-administration or co-localization of the mEVs (such as smEVs and/or pmEVs) from the two strains.

“Dysbiosis” refers to a state of the microbiota or microbiome of the gut or other body area, including, e.g., mucosal or skin surfaces (or any other microbiome niche) in which the normal diversity and/or function of the host gut microbiome ecological networks (“microbiome”) are disrupted. A state of dysbiosis may result in a diseased state, or it may be unhealthy under only certain conditions or only if present for a prolonged period. Dysbiosis may be due to a variety of factors, including, environmental factors, infectious agents, host genotype, host diet and/or stress. A dysbiosis may result in, a change (e.g., increase or decrease) in the prevalence of one or more bacteria types (e.g., anaerobic), species and/or strains, change (e.g., increase or decrease) in diversity of the host microbiome population composition; a change (e.g., increase or reduction) of one or more populations of symbiont organisms resulting in a reduction or loss of one or more beneficial effects; overgrowth of one or more populations of pathogens (e.g., pathogenic bacteria); and/or the presence of, and/or overgrowth of, symbiotic organisms that cause disease only when certain conditions are present.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state. Properties that may be decreased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size (e.g., in an animal tumor model)).

The term “ecological consortium” is a group of bacteria which trades metabolites and positively co-regulates one another, in contrast to two bacteria which induce host synergy through activating complementary host pathways for improved efficacy.

As used herein, “engineered bacteria” are any bacteria that have been genetically altered from their natural state by human activities, and the progeny of any such bacteria. Engineered bacteria include, for example, the products of targeted genetic modification, the products of random mutagenesis screens and the products of directed evolution.

The term “epitope” means a protein determinant capable of specific binding to an antibody or T cell receptor. Epitopes usually consist of chemically active surface groupings of molecules such as amino acids or sugar side chains. Certain epitopes can be defined by a particular sequence of amino acids to which an antibody is capable of binding.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Nat. Acad. Sci. USA 85:2444 (other programs include the GCG program package (Devereux, J., el al., Nucleic Acids Research 12(1):387 (1984)), BLASTP. BLASTN. FASTA Atschul, S. F., et al., J Molec Biol 215:403 (1990); Guide to Huge Computers, Mrtin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison. Wis.) and the University of Wisconsin Genetics Computer Group (UWG) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (e.g., psoriasis, atopic dermatitis, lupus, scleroderma, hemolytic anemia, vasculitis, type one diabetes, Grave's disease, rheumatoid arthritis, multiple sclerosis, Goodpasture's syndrome, pernicious anemia and/or myopathy), inflammatory diseases (e.g., acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (e.g., food allergies, drug allergies and/or environmental allergies).

“Immunotherapy” is treatment that uses a subject's immune system to treat disease (e.g., immune disease, inflammatory disease, metabolic disease, cancer) and includes, for example, checkpoint inhibitors, cancer vaccines, cytokines, cell therapy. CAR-T cells, and dendritic cell therapy.

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10\hat{ }3 fold, 10\hat{ }4 fold, 10\hat{ }5 fold, 10\hat{ }6 fold, and/or 10\hat{ }7 fold greater after treatment when compared to a pre-treatment state. Properties that may be increased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size (e.g., in an animal tumor model).

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant, immuno-adjuvants are a specific class of broader adjuvant or adjuvant therapy. Examples of STING agonists include, but are not limited to, 2′3′-cGAMP, 3′3′-cGAMP, c-di-AMP, c-di-GMP, 2′2′-cGAMP, and 2′3′-cGAM(PS)2 (Rp/Sp) (Rp, Sp-isomers of the bis-phosphorothioate analog of 2′3′-cGAMP) Examples of TLR agonists include, but am not limited to, TLR1, TLR2, TLR3, TLR4, TLR5, TLR6, TLR7, TLR8, TLR9, TLR1O and TLRI 1. Examples of NOD agonists include, but are not limited to, N-acetylmuramyl-L-alanyl-D-isoglutamine (muramyldipeptide (MDP)), gamma-D-glutamyl-meso-diaminopimelic acid (iE-DAP), and desmuramylpeptides (DMP).

The “internal transcribed spacer” or “ITS” is a piece of non-functional RNA located between structural ribosomal RNAs (rRNA) on a common precursor transcript often used for identification of eukaryotic species in particular fungi. The rRNA of fungi that forms the core of the ribosome is transcribed as a signal gene and consists of the 8S, 5.8S and 28S regions with ITS4 and 5 between the 8S and 5.8S and 5.8S and 28S regions, respectively. These two intercistronic segments between the 18S and 5.8S and 5.8S and 28S regions are removed by splicing and contain significant variation between species for barcoding purposes as previously described (Schoch et al Nuclear ribosomal internal transcribed spacer (ITS) region as a universal DNA barcode marker for Fungi. PNAS 109:6241-6246. 2012). 18S rDNA is traditionally used for phylogenetic reconstruction however the ITS can serve this function as it is generally highly conserved but contains hypervariable regions that harbor sufficient nucleotide diversity to differentiate genera and species of most fungus.

The term “isolated” or “enriched” encompasses a microbe, an mEV (such as an smEV and/or pmEV) or other entity or substance that has been (1) separated from at least some of the components with which it was associated when initially produced (whether in nature or in an experimental setting), and/or (2) produced, prepared, purified, and/or manufactured by the hand of man. Isolated microbes or mEVs may be separated from at least about 10%, about 20%, about 30%, about 40%, about 50%, about 60%, about 70%, about 80%, about 90%, or more of the other components with which they were initially associated. In some embodiments, isolated microbes or mEVs are more than about 80%, about 85%, about 90%, about 91%, about 92%, about 93%, about 94%, about 95%, about 96%, about 97%, about 98%, about 99%, or more than about 99% pure, e.g., substantially free of other components. The terms “purify,” “purifying” and “purified” refer to a microbe or mEV or other material that has been separated from at least some of the components with which it was associated either when initially produced or generated (e.g., whether in nature or in an experimental setting), or during any time after its initial production. A microbe or a microbial population or mEV may be considered purified if it is isolated at or after production, such as from a material or environment containing the microbe or microbial population or mEV, and a purified microbe or microbial or mEV population may contain other materials up to about 10%, about 20%, about 30%, about 40%, about 50%, about 60%, about 70%, about 80%, about 90%, or above about 90% and still be considered “isolated.” In some embodiments, purified microbes or mEVs or microbial population are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components such as mEVs thereof are generally purified from residual habitat products.

As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans).

The term “LPS mutant or lipopolysaccharide mutant” broadly refers to selected bacteria that comprises loss of LPS. Loss of LPS might be due to mutations or disruption to genes involved in lipid A biosynthesis, such as lpxA, lpxC, and lpxD. Bacteria comprising LPS mutants can be resistant to aminoglycosides and polymyxins (polymyxin B and colistin).

“Metabolite” as used herein refers to any and all molecular compounds, compositions, molecules, ions, co-factors, catalysts or nutrients used as substrates in any cellular or microbial metabolic reaction or resulting as product compounds, compositions, molecules, ions, co-factors, catalysts or nutrients from any cellular or microbial metabolic reaction.

“Microbe” refers to any natural or engineered organism characterized as a archaeaon, parasite, bacterium, fungus, microscopic alga, protozoan, and the stages of development or life cycle stages (e.g., vegetative, spore (including sporulation, dormancy, and germination), latent, biofilm) associated with the organism. Examples of gut microbes include: Actinomyces graevenitzii, Actinomyces odontolyticus, Akkermansia muciniphila. Bacteroides caccae, Bacteroides fragilis, Bacteroides putredinis, Bacteroides thetaiotaomicron, Bacteroides vultagus, Bifidobacterium adolescentis, Bfidobacterium bifidum, Bilophila wadsworthia, Blautia, Butyrivibrio, Campylobacter gracilis, Clostridia cluster III, Clostridia cluster IV, Clostridia cluster IX (Acidaminococcaceae group), Clostridia cluster XI, Clostridia cluster XIII (Peptostreptococcus group), Clostridia cluster XIV, Clostridia cluster XV, Collinsella aerofaciens, Coprococcus, Corynebacterium sunsvallense, Desulfomonas pigra, Dorea formicigenerans, Dorea longicatena, Escherichia coli, Eubacterium hadrum, Eubacterium rectale, Faecalibacteria prausnitzii, Gemella, Lactococcus, Lanchnospira, Mollicutes cluster XVI, Mollicutes cluster XVII, Prevotella, Rothia mucilaginosa, Ruminococcus callidus, Ruminococcus gnavus, Ruminococcus torques, and Streptococcus.

“Microbial extracellular vesicles” (mEVs) can be obtained from microbes such as bacteria, archaea, fungi, microscopic algae, protozoans, and parasites. In some embodiments, the mEVs are obtained from bacteria. mEVs include secreted microbial extracellular vesicles (smEVs) and processed microbial extracellular vesicles (pmEVs). “Secreted microbial extracellular vesicles” (smEVs) are naturally-produced vesicles derived from microbes. smEVs are comprised of microbial lipids and/or microbial proteins and/or microbial nucleic acids and/or microbial carbohydrate moieties, and are isolated from culture supernatant. The natural production of these vesicles can be artificially enhanced (e.g., increased) or decreased through manipulation of the environment in which the bacterial cells are being cultured (e.g., by media or temperature alterations). Further, smEV compositions may be modified to reduce, increase, add, or remove microbial components or foreign substances to alter efficacy, immune stimulation, stability, immune stimulatory capacity, stability, organ targeting (e.g., lymph node), absorption (e.g., gastrointestinal), and/or yield (e.g., thereby altering the efficacy). As used herein, the term “purified smEV composition” or “smEV composition” refers to a preparation of smEVs that have been separated from at least one associated substance found in a source material (e.g., separated from at least one other microbial component) or any material associated with the smEVs in any process used to produce the preparation. It can also refer to a composition that has been significantly enriched for specific components. “Processed microbial extracellular vesicles” (pmEVs) are a non-naturally-occurring collection of microbial membrane components that have been purified from artificially lysed microbes (e.g., bacteria) (e.g., microbial membrane components that have been separated from other, intracellular microbial cell components), and which may comprise particles of a varied or a selected size range, depending on the method of purification. A pool of pmEVs is obtained by chemically disrupting (e.g., by lysozyme and/or lysostaphin) and/or physically disrupting (e.g., by mechanical force) microbial cells and separating the microbial membrane components from the intracellular components through centrifugation and/or ultracentrifugation, or other methods. The resulting pmEV mixture contains an enrichment of the microbial membranes and the components thereof (e.g., peripherally associated or integral membrane proteins, lipids, glycans, polysaccharides, carbohydrates, other polymers), such that there is an increased concentration of microbial membrane components, and a decreased concentration (e.g., dilution) of intracellular contents, relative to whole microbes. For gram-positive bacteria, pmEVs may include cell or cytoplasmic membranes. For gram-negative bacteria, a pmEV may include inner and outer membranes. pmEVs may be modified to increase purity, to adjust the size of particles in the composition, and/or modified to reduce, increase, add or remove, microbial components or foreign substances to alter efficacy, immune stimulation, stability, immune stimulatory capacity, stability, organ targeting (e.g., lymph node), absorption (e.g., gastrointestinal), and/or yield (e.g., thereby altering the efficacy). pmEVs can be modified by adding, removing, enriching for, or diluting specific components, including intracellular components from the same or other microbes. As used herein, the term “purified pmEV composition” or “pmEV composition” refers to a preparation of pmEVs that have been separated from at least one associated substance found in a source material (e.g., separated from at least one other microbial component) or any material associated with the pmEVs in any process used to produce the preparation. It can also refer to a composition that has been significantly enriched for specific components.

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state microbiome. The microbiome may be native to the subject or patient, or components of the microbiome may be modulated, introduced, or depleted due to changes in health state (e.g., precancerous or cancerous state) or treatment conditions (e.g., antibiotic treatment, exposure to different microbes). In some aspects, the microbiome occurs at a mucosal surface. In some aspects, the microbiome is a gut microbiome. In some aspects, the microbiome is a tumor microbiome.

A “microbiome profile” or a “microbiome signature” of a tissue or sample refers to an at least partial characterization of the bacterial makeup of a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present or absent in a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more cancer-associated bacterial strains are present in a sample. In some embodiments, the microbiome profile indicates the relative or absolute amount of each bacterial strain detected in the sample. In some embodiments, the microbiome profile is a cancer-associated microbiome profile. A cancer-associated microbiome profile is a microbiome profile that occurs with greater frequency in a subject who has cancer than in the general population. In some embodiments, the cancer-associated microbiome profile comprises a greater number of or amount of cancer-associated bacteria than is normally present in a microbiome of an otherwise equivalent tissue or sample taken from an individual who does not have cancer.

“Modified” in reference to a bacteria broadly refers to a bacteria that has undergone a change from its wild-type form. Bacterial modification can result from engineering bacteria. Examples of bacterial modifications include genetic modification, gene expression modification, phenotype modification, formulation modification, chemical modification, and dose or concentration. Examples of improved properties are described throughout this specification and include, e.g., attenuation, auxotrophy, homing, or antigenicity. Phenotype modification might include, by way of example, bacteria growth in media that modify the phenotype of a bacterium such that it increases or decreases virulence.

An “oncobiome” as used herein comprises tumorigenic and/or cancer-associated microbiota, wherein the microbiota comprises one or more of a virus, a bacterium, a fungus, a protist, a parasite, or another microbe.

“Oncotrophic” or “oncophilic” microbes and bacteria are microbes that are highly associated or present in a cancer microenvironment. They may be preferentially selected for within the environment, preferentially grow in a cancer microenvironment or hone to a said environment.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share ≥97% average nucleotide identity across the entire 16S or some variable region of the 16S are considered the same OTU. See e.g., Claesson M J. Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940. For complete genomes, MLSTs, specific genes, other than 16S, or sets of genes OTUs that share ≥95% average nucleotide identity are considered the same OTU. See e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940. OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic clade are provided herein.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a substance is “pure” if it is substantially free of other components. The terms “purify,” “purifying” and “purified” refer to an mEV (such as an smEV and/or a pmEV) preparation or other material that has been separated from at least some of the components with which it was associated either when initially produced or generated (e.g., whether in nature or in an experimental setting), or during any time after its initial production. An mEV (such as an smEV and/or a pmEV) preparation or compositions may be considered purified if it is isolated at or after production, such as from one or more other bacterial components, and a purified microbe or microbial population may contain other materials up to about 10%, about 20%, about 30%, about 40%, about 50%, about 60%, about 70%, about 80%, about 90%, or above about 90% and still be considered “purified.” In some embodiments, purified mEVs (such as smEVs and/or pmEVs) are more than about 80%, about 85%, about 90%, about 91%, about 92%, about 93%, about 94%, about 95%, about 96%, about 97%, about 98%, about 99%, or more than about 99% pure. mEV (such as an smEV and/or a pmEV) compositions (or preparations) are, e.g., purified from residual habitat products.

As used herein, the term “purified mEV composition” or “mEV composition” refers to a preparation that includes mEVs (such as smEVs and/or pmEVs) that have been separated from at least one associated substance found in a source material (e.g., separated from at least one other bacterial component) or any material associated with the mEVs (such as smEVs and/or pmEVs) in any process used to produce the preparation. It also refers to a composition that has been significantly enriched or concentrated. In some embodiments, the mEVs (such as smEVs and/or pmEVs) are concentrated by 2 fold, 3-fold, 4-fold, 5-fold, 10-fold, 100-fold, 1000-fold, 10,000-fold or more than 10,000 fold.

“Residual habitat products” refers to material derived from the habitat for microbiota within or on a subject. For example, fermentation cultures of microbes can contain contaminants, e.g., other microbe strains or forms (e.g., bacteria, virus, mycoplasm, and/or fungus). For example, microbes live in feces in the gastrointestinal tract, on the skin itself, in saliva, mucus of the respiratory tract, or secretions of the genitourinary tract (i.e., biological matter associated with the microbial community). Substantially free of residual habitat products means that the microbial composition no longer contains the biological matter associated with the microbial environment on or in the culture or human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the microbial composition contains no detectable cells from a culture contaminant or a human or animal and that only microbial cells are detectable. In one embodiment, substantially free of residual habitat products may also mean that the microbial composition contains no detectable viral (including bacteria, viruses (e.g., phage)), fungal, mycoplasmal contaminants. In another embodiment, it means that fewer than 1×10⁻²%, 1×10⁻³%, 1×10⁻⁴%, 1×10⁻⁵%, 1×10⁻⁶%, 1×10⁻⁷%, 1×10⁻⁸% of the viable cells in the microbial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (e.g., PCR, DNA sequencing), scrology and antigen analysis, enzymatic and metabolic analysis, and methods using instrumentation such as flow cytometry with reagents that distinguish desired constituents from contaminants.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein). Alternatively, specific binding applies more broadly to a two component system where one component is a protein, lipid, or carbohydrate or combination thereof and engages with the second component which is a protein, lipid, carbohydrate or combination thereof in a specific way.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

The terms “subject” or “patient” refers to any mammal. A subject or a patient described as “in need thereof” refers to one in need of a treatment (or prevention) for a disease. Mammals (i.e., mammalian animals) include humans, laboratory animals (e.g., primates, rats, mice), livestock (e.g., cows, sheep, goats, pigs), and household pets (e.g., dogs, cats, rodents). The subject may be a human. The subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject may be healthy, or may be suffering from a cancer at any developmental stage, wherein any of the stages are either caused by or opportunistically supported of a cancer associated or causative pathogen, or may be at risk of developing a cancer, or transmitting to others a cancer associated or cancer causative pathogen. In some embodiments, a subject has lung cancer, bladder cancer, prostate cancer, plasmacytoma, colorectal cancer, rectal cancer. Merkel Cell carcinoma, salivary gland carcinoma, ovarian cancer, and/or melanoma. The subject may have a tumor. The subject may have a tumor that shows enhanced macropinocytosis with the underlying genomics of this process including Ras activation. In other embodiments, the subject has another cancer. In some embodiments, the subject has undergone a cancer therapy.

As used herein, the term “treating” a disease in a subject or “treating” a subject having or suspected of having a disease refers to administering to the subject to a pharmaceutical treatment, e.g., the administration of one or more agents, such that at least one symptom of the disease is decreased or prevented from worsening. Thus, in one embodiment. “treating” refers inter alia to delaying progression, expediting remission, inducing remission, augmenting remission, speeding recovery, increasing efficacy of or decreasing resistance to alternative therapeutics, or a combination thereof. As used herein, the term “preventing” a disease in a subject refers to administering to the subject to a pharmaceutical treatment, e.g., the administration of one or more agents, such that onset of at least one symptom of the disease is delayed or prevented.

As used herein, a “type” of bacteria may be distinguished from other bacteria by: genus, species, sub-species, strain or by any other taxonomic categorization, whether based on morphology, physiology, genotype, protein expression or other characteristics known in the art.

### Bacteria

in certain aspects, provided herein are pharmaceutical compositions that comprise mEVs (such as smEVs and/or pmEVs) obtained from Oscillospiraceae bacteria.

In some embodiments, the pharmaceutical composition comprises mEVs (such as smEVs and/or pmEVs) and the mEVs (such as smEVs and/or pmEVs) are produced from a high yield strain of Oscillospiraceae bacteria. In some embodiments, the high yield strain produces at least 3×10¹³ mEVs (such as smEVs and/or pmEVs) per liter from a bioreactor-grown culture.

In some embodiments, the Oscillospiraceae bacteria from which the mEVs (such as smEVs and/or pmEVs) are obtained are modified to reduce toxicity or other adverse effects, to enhance delivery) (e.g., oral delivery) of the mEVs (such as smEVs and/or pmEVs) (e.g., by improving acid resistance, muco-adherence and/or penetration and/or resistance to bile acids, digestive enzymes, resistance to anti-microbial peptides and/or antibody neutralization), to target desired cell types (e.g., M-cells, goblet cells, enterocytes, dendritic cells, macrophages), to enhance their immunomodulatory and/or therapeutic effect of the mEVs (such as smEVs and/or pmEVs) (e.g., either alone or in combination with another therapeutic agent), and/or to enhance immune activation or suppression by the mEVs (such as smEVs and/or pmEVs) (e.g., through modified production of polysaccharides, pili, fimbriae, adhesins). In some embodiments, the engineered Oscillospiraceae bacteria described herein are modified to improve mEV (such as smEV and/or pmEV) manufacturing (e.g., higher oxygen tolerance, stability, improved freeze-thaw tolerance, shorter generation times). For example, in some embodiments, the engineered Oscillospiraceae bacteria described include bacteria harboring one or more genetic changes, such change being an insertion, deletion, translocation, or substitution, or any combination thereof, of one or more nucleotides contained on the bacterial chromosome or endogenous plasmid and/or one or more foreign plasmids, wherein the genetic change may results in the overexpression and/or underexpression of one or more genes. The engineered Oscillospiraceae bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, or any combination thereof.

Species and/or strains of Oscillospiraceae bacteria can be used as a source of mEVs (such as smEVs and/or pmEVs). In certain embodiments, mEVs are generated from one Oscillospiraceae bacterial strain. In certain embodiments, mEVs are generated from a combination of Oscillospiraceae bacterial strains. In some embodiments, the combination is a combination of at least 2, 3, 4, 5, 6, 7, 8, 9, 10, 11, 12, 13, 14, 15, 20, 25, 30, 35, 40, 45 or 50 Oscillospiraceae bacterial strains. In some embodiments, Oscillospiraceae strain is Faecalibacterium prausnitzii (e.g., Faecalibacterium prausnitzii strain A), Fournierella massiliensis (e.g., Fournierella massiliensis strain A), Harryflintia acetispora (e.g., Harryflintia acetispora strain A), Agathobaculum sp. (e.g., Agathobaculum sp. strain A), Acutalibacter sp. (e.g., Acutalibacter sp. strain A). Anaerotruncus colihominis (Anaerotruncus colihominis strain A), or Subdoligranulum variable (e.g., Subdoligranulum variabile strain A).

Examples of strains of Oscillospiraceae bacteria that can be used as a source of mEVs (such as smEVs and/or pmEVs) described herein are provided in Table 2 and elsewhere throughout the specification. In some embodiments, the Oscillospiraceae bacterial strain is a bacterial strain having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain listed in Table 2. In certain embodiments, mEVs are generated from a single strain of Oscillospiraceae bacterial strains provided herein. In certain embodiments, mEVs are generated from a combination of Oscillospiraceae bacterial strains provided herein. In some embodiments, the combination is a combination of at least 2, 3, 4, 5, 6, 7, 8, 9, 10, 11, 12, 13, 14, 15, 20, 25, 30, 35, 40, 45 or 50 bacterial strains. In some embodiments, the combination includes mEVs from bacterial strains listed in Table 1 and/or bacterial strains having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain listed in Table 2.

In some embodiments, the mEVs (such as smEVs and/or pmEVs) described herein are obtained from a strain of Oscillospiraceae bacteria comprising a genomic sequence that is at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the genomic sequence of the strain of bacteria deposited with the ATCC Deposit number as provided in Table 2. In some embodiments, the mEVs (such as smEVs and/or pmEVs) described herein are obtained from a strain of Oscillospiraceae bacteria comprising a 16S sequence that is at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the 16S sequence as provided in Table 2.

TABLE 2
Exemplary Oscillospiraceae Bacterial Strains

| SEQ ID | | Deposit | |
|---|---|---|---|
| No. | Strain | Number | 16S Sequence |
| 1 | Faecalibacterium | PTA-126792 | > Faecalibacterium |
| | prausnitzii | | prausnitzii 16S |
| | Strain A | | Ribosomal RNA Sequence |
| | | | TATAAAGAGTTTGATCCTGGCTCAGG |
| | | | ACGAACGCTGGCGGCGCGCCTAACAC |
| | | | ATGCAAGTCGAACGAGCGAGAGAGAG |
| | | | CTTGCTTTCTCGAGTGAGTGGCGAAC |
| | | | GGGTGAGTAACGCGTGAGGAACCTGC |
| | | | CTCAAAGAGGGGGACAACAGTTGGAA |
| | | | ACGACTGCTAATACCGCATAAGCCCA |
| | | | CGACCCGGCATCGGGTAGAGGGAAAA |
| | | | GGAGCAATCCGCTTTGAGATGGCCTC |
| | | | GCGTCCGATTAGCTAGTTGGTGAGGT |
| | | | AACGGCCCACCAAGGCGACGATCGGT |
| | | | AGCCGGACTGAGAGGTTGAACGGCCA |
| | | | CATTGGGACTGAGACACGGCCCAGAC |
| | | | TCCTACGGGAGGCAGCAGTGGGGAAT |
| | | | ATTGCACAATGGGGGAAACCCTGATG |
| | | | CAGCGACGCCGCGTGGAGGAAGAAGG |
| | | | TCTTCGGATTGTAAACTCCTGTTGTT |
| | | | GAGGAAGATAATGACGGTACTCAACA |
| | | | AGGAAGTGACGGCTAACTACGTGCCA |
| | | | GCAGCCGCGGTAAAACGTAGGTCACA |
| | | | AGCGTTGTCCGGAATTACTGGGTGTA |
| | | | AAGGGAGCGCAGGCGGGAAGACAAGT |
| | | | TGGAAGTGAAATCCATGGGCTCAACC |
| | | | CATGAACTGCTTTCAAAACTGTTTTT |
| | | | CTTGAGTAGTGCAGAGGTAGGCGGAA |
| | | | TTCCCGGTGTAGCGGTGGAATGCGTA |
| | | | GATATCGGGAGGAACACCAGTGGCGA |
| | | | AGGCGGCCTACTGGGCACCAACTGAC |
| | | | GCTGAGGCTCGAAAGTGTGGGTAGCA |
| | | | AACAGGATTAGATACCCTGGTAGTCC |
| | | | ACACTGTAAACGATGATTACTAGGTG |
| | | | TTGGAGGATTGACCCCTTCAGTGCCT |
| | | | GGGGAGTACGACCGCAAGGTTGAAAC |
| | | | TCAAAGGAATTGACGGGGGCCCGCAC |
| | | | AAGCAGTGGAGTATGTGGTTTAATTC |
| | | | GACGCAACGCGAAGAACCTTACCAAG |
| | | | TCTTGACATCCTGCGACGGTGCTGGA |
| | | | AACAGTACTTTCCTTCGGGACGCAGA |
| | | | GACAGGTGGTGCATGGTTGTCGTCAG |
| | | | CTCGTGTCGTGAGATGTTGGGTTAAG |
| | | | TCCCGCAACGAGCGCAACCCTTATGG |
| | | | TCAGTTACTACGCAAGAGGACTCTGG |
| | | | CCAGACTGCCGTTGACAAAACGGAGG |
| | | | AAGGTGGGGATGACGTCAAATCATCA |
| | | | TGCCCTTTATGACTTGGGCTACACAC |
| | | | GTACTACAATGGCGTTAAACAAAGAG |
| | | | AAGCAAGACCGCGAGGTGGAGCAAAA |
| | | | CTCAGAAACAACGTCCCAGTTCGGAC |
| | | | TGCAGGCTGCAACTCGCCTGCACGAA |
| | | | GTCGGAATTGCTAGTAATCGCAGATC |
| | | | AGCATGCTGCGGTGAATACGTTCCCG |
| | | | GGCCTTGTACACACCGCCCGTCACAC |
| | | | CATGAGAGCCGGGGGGACCCGAAGTC |
| | | | GGTAGTCTAACCGCAAGGAGGACGCC |
| | | | GCCGAAGGTAAAACTGGTGATTGGGG |
| | | | TGAAGTCGTAACAAGGTAGCCGTAGG |
| | | | AGAACCTGCGGCTGGATCACCTCCTT |
| | | | T |
| 2 | Fournierella | PTA-126696 | > Fournierella |
| | massiliensis | | massiliensis 16S |
| | strain A | | Ribosomal RNA Sequence |
| | | | TATGAAGAGTITGATCCTGGCTCAGG |
| | | | ACGAACGCTGGCGGCGCGCCTAACAC |
| | | | ATGCAAGTCGAACGGAGCTTGCTTGT |
| | | | CAGATCCTTTCGGGGTGACGACTIGT |
| | | | AAGCTTAGTGGCGAACGGGTGAGTAA |
| | | | CACGTGAGTAACCTGCCCCAGAGTGG |
| | | | GGGACAACAGTTGGAAACGACTGCTA |
| | | | ATACCGCATAAGCCCACGGAACCGCA |
| | | | TGGTTCTGAGGGAAAAGGAGCAATTC |
| | | | GCTTTGGGATGGACTCGCGTCCGATT |
| | | | AGCTAGATGGTGAGGTAACGGCCCAC |
| | | | CATGGCGACGATCGGTAGCCGGACTG |
| | | | AGAGGTTGATCGGCCACATTGGGACT |
| | | | GAGACACGGCCCAGACTCCTACGGGA |
| | | | GGCAGCAGTGGGGAATATTGCACAAT |
| | | | GGGGGAAACCCTGATGCAGCGACGCC |
| | | | GCGTGGAGGAAGAAGGCCTTCGGGTT |
| | | | GTAAACTCCTGTCGTAAGGGACGATA |
| | | | GTGACGGTACCTTACAAGAAAGCCAC |
| | | | GGCTAACTACGTGCCAGCAGCCGCGG |
| | | | TAAAACGTAGGTGGCAAGCGTTGTCC |
| | | | GGAATTACTGGGTGTAAAGGGAGCGC |
| | | | AGGCGGGTCTGCAAGTIGGAAGTGAA |
| | | | ACTCATGGGCTCAACCCATGAACTGC |
| | | | TTTCAAAACTGCGGATCTTGAGTGGT |
| | | | GTAGAGGTAGGCGGAATTCCCGGTGT |
| | | | AGCGGTGGAATGCGTAGATATCGGGA |
| | | | GGAACACCAGTGGCGAAGGCGGCCTA |
| | | | CTGGGCACTAACTGACGCTGAGGCTC |
| | | | GAAAGCATGGGTAGCAAACAGGATTA |
| | | | GATACCCTGGTAGTCCATGCCGTAAA |
| | | | CGATGATTACTAGGIGTGGGAGGATT |
| | | | GACCCCTTCCGTGCCGCAGTTAACAC |
| | | | AATAAGTAATCCACCTGGGGAGTACG |
| | | | ACCGCAAGGTIGAAACTCAAAGGAAT |
| | | | TGACGGGGGCCCGCACAAGCAGTGGA |
| | | | GTATGTGGTTTAATTCGAAGCAACGC |
| | | | GAAGAACCTTACCAGGTCTTGACATC |
| | | | CGATGCATAGIGTAGAGATACATGAA |
| | | | GTCCTTCGGGACATCGAGACAGGTGG |
| | | | TGCATGGTTGTCGTCAGCTCGTGTCG |
| | | | TGAGATGTTGGGTTAAGTCCCGCAAC |
| | | | GAGCGCAACCCTTATCGTTAGTTACT |
| | | | ACGCAAGAGGACTCTAGCGAGACTGC |
| | | | CGTTGACAAAACGGAGGAAGGTGGGG |
| | | | ATGACGTCAAATCATCATGCCCTTTA |
| | | | TGACCTGGGCTACACACGTACTACAA |
| | | | TGGCAATTAACAAAGAGAAGCGAAGC |
| | | | CGCGAGGTGGAGCAAACCTCATAAAA |
| | | | ATTGTCTCAGTTCAGATIGCAGGCTG |
| | | | CAACTCGCCTGCATGAAGTCGGAATT |
| | | | GCTAGTAATCGCGGATCAGCATGCCG |
| | | | CGGTGAATACGTTCCCGGGCCTTGTA |
| | | | CACACCGCCCGTCACACCATGAGAGC |
| | | | CGGGGGGACCCGAAGTCCGTAGCCTA |
| | | | ACCGCAAGGAGGGCGCGGCCGAAGGT |
| | | | AAAACTGGTGATTGGGGTGAAGTCGT |
| | | | AACAAGGTAGCCGTATCGGAAGGTGC |
| | | | GGCTGGATCACCTCCTTT |
| 3 | Harryflintia | PTA-126694 | > Harryflintia acetispora |
| | acetispora | | 16S Ribosomal RNA Sequence |
| | strain A | | CTAAAGAGTTTGATCCTGGCTCAGGA |
| | | | CGAACGCTGGCGGCGCGCCTAACACA |
| | | | TGCAAGTCGAACGGAGAAATGCTGAG |
| | | | CTTGCTTTGCATTTCTTAGTGGCGGA |
| | | | CGGGTGAGTAACACGTGAGCAACCTG |
| | | | CCTTTGTGAGGGGAATAACGTCTGGA |
| | | | AACGGACGCTAATACCGCATAACGTC |
| | | | AAGGAACCGCATGGTTTTTTGACCAA |
| | | | AGATTTTATCGCACAAAGATGGGCTC |
| | | | GCGGCTGATTAGCTAGTTGGCGGGGT |
| | | | AACGGCCCACCAAGGCGACGATCAGT |
| | | | AGCCGGACTGAGAGGTTGATCGGCCA |
| | | | CATTGGGACTGAGACACGGCCCAGAC |
| | | | TCCTACGGGAGGCAGCAGTGGGGGAT |
| | | | ATTGCACAATGGGGGAAACCCTGATG |
| | | | CAGCGACGCCGCGTGAGGGAAGACGG |
| | | | TTTTCGGATTGTAAACCTCTGTCTTC |
| | | | AGGGACGAAATCAATGACGGTACCTG |
| | | | AGGAGGAAGCCACGGCTAACTACGTG |
| | | | CCAGCAGCCGCGGTAATACGTAGGTG |
| | | | GCAAGCGTTGTCCGGAATTACTGGGT |
| | | | GTAAAGGGAGCGTAGGCGGGAATGCA |
| | | | AGTTGAATGTTTAAACTATCGGCTCA |
| | | | ACTGATAATCGCGTTCAAAACTGCAT |
| | | | TTCTTGAGTGGAGTAGAGGCAGGCGG |
| | | | AATTCCTAGTGTAGCGGTGAAATGCG |
| | | | TAGATATTAGGAGGAACACCAGTGGC |
| | | | GAAGGCGGCCTGCTGGGCTCTAACTG |
| | | | ACGCTGAGGCTCGAAAGCGTGGGTAG |
| | | | CAAACAGGATTAGATACCCTGGTAGT |
| | | | CCACGCCGTAAACGATGATTACTAGG |
| | | | TGTGGGGGGACTGACCCCTTCCGTGC |
| | | | CGGAGTTAACACAATAAGTAATCCAC |
| | | | CTGGGGAGTACGGTCOCAAGACTGAA |
| | | | ACTCAAAGGAATTGACGGGGGCCCGC |
| | | | ACAAGCAGTGGAGTATGTGGTTTAAT |
| | | | TCGAAGCAACGCGAAGAACCTTACCA |
| | | | GGTCTTGACATCGTGCGCATACCGTA |
| | | | GAGATACGGGAAGTCCTTCGGGACGC |
| | | | ATAGACAGGTGGTGCATGGTTGTCGT |
| | | | CAGCTCGTGTCGTGAGATGTTGGGTT |
| | | | AAGTCCCGCAACGAGCGCAACCCTTA |
| | | | TTATTAGTTGCTACGCAAGAGCACTC |
| | | | TAATGAGACTGCCGTTGACAAAACGG |
| | | | AGGAAGGTGGGGATGACGTCAAATCA |
| | | | TCATGCCCCTTATGACCTGGGCTACA |
| | | | CACGTACTACAATGGCACTTAACAGA |
| | | | GGGAAGCAAGACCGCGAGGTGGAGCA |
| | | | AACCCCCAAAAAGTGTCTCAGTTCGG |
| | | | ATTGCAGGCTGCAACCCGCCTGTATG |
| | | | AAGTCGGAATTGCTAGTAATCGCGGA |
| | | | TCAGCATGCCGCGGTGAATACGTTCC |
| | | | CGGGCCTTGTACACACCGCCCGTCAC |
| | | | ACCATGAGAGCCGGTAACACCCGAAG |
| | | | TCAGTAGCCTAACCGCAAGGAGGGCG |
| | | | CTGCCGAAGGTGGGATTGGTGATTAG |
| | | | GGTGAAGTCGTAACAAGGTAGCCGTA |
| | | | TCGGAAGGTGCGGCTGGATCACCTCC |
| | | | TTT |
| 4 | Agathobaculum | PTA-125892 | > Agathobaculum sp. |
| | sp. strain A | | 16S Ribosomal RNA |
| | | | TTTAGAGAGTTTGATCCTGGCTCAGG |
| | | | ATGAACGCTGGCGGCGTGCCTAACAC |
| | | | ATGCAAGTCGAACGGAGTTATTTTGG |
| | | | AAATCTCTTCGGAGATGGAATCTTTA |
| | | | ACTTAGTGGCGGACGGGTGAGTAACG |
| | | | CGTGAGCAATCTGCCTTTAAGAGGGG |
| | | | GATAACAGTCGGAAACGGCTGCTAAT |
| | | | ACCGCATAAAGCATTGAATTCGCATG |
| | | | TTTTCGATGCCAAAGGAGCAATCCGC |
| | | | TTTTAGATGAGCTCGCGTCTGATTAG |
| | | | CTAGTTGGCGGGGTAACGGCCCACCA |
| | | | AGGCGACGATCAGTAGCCGGACTGAG |
| | | | AGGTTGAACGGCCACATTGGGACTGA |
| | | | GACACGGCCCAGACTCCTACGGGAGG |
| | | | CAGCAGTGGGGAATATTGCGCAATGG |
| | | | GGGAAACCCTGACGCAGCAACGCCGC |
| | | | GTGATTGAAGAAGGCCTTCGGGTTGT |
| | | | AAAGATCTTTAATTCGGGACGAAAAA |
| | | | TGACGGTACCGAAAGAATAAGCTCCG |
| | | | GCTAACTACGTGCCAGCAGCCGCGGT |
| | | | AATACGTAGGGAGCAAGCGTTATCCG |
| | | | GATTTACTGGGTGTAAAGGGCGCGCA |
| | | | GGCGGGCTGGCAAGTTGGAAGTGAAA |
| | | | TCTAGGGGCTTAACCCCTAAACTGCT |
| | | | TTCAAAACTGCTGGTCTTGAGTGATG |
| | | | GAGAGGCAGGCGGAATTCCGTGTGTA |
| | | | GCGGTGAAATGCGTAGATATACGGAG |
| | | | GAACACCAGTGGCGAAGGCGGCCTGC |
| | | | TGGACATTAACTGACGCTGAGGCGCG |
| | | | AAAGCGTGGGGAGCAAACAGGATTAG |
| | | | ATACCCTGGTAGTCCACGCCGTAAAC |
| | | | GATGGATACTAGGTGTGGGAGGTATT |
| | | | GACCCCTTCCGTGCCGCAGTTAACAC |
| | | | AATAAGTATCCCACCTGGGGAGTACG |
| | | | GCCGCAAGGTTGAAACTCAAAGGAAT |
| | | | TGACGGGGGCCCGCACAAGCAGTGGA |
| | | | GTATGTGGTTTAATTCGAAGCAACGC |
| | | | GAAGAACCTTACCAGGCCTTGACATC |
| | | | CCGATGACCGGTCTAGAGATAGACCT |
| | | | TCTCTTCGGAGCATCGGTGACAGGTG |
| | | | GTGCATGGTTGTCGTCAGCTCGTGTC |
| | | | GTGAGATGTTGGGTTAAGTCCCGCAA |
| | | | CGAGCGCAACCCTTACGGTTAGTTGA |
| | | | TACGCAAGATCACTCTAGCCGGACTG |
| | | | CCGTTGACAAAACGGAGGAAGGTGGG |
| | | | GACGACGTCAAATCATCATGCCCCTT |
| | | | ATGGCCTGGGCTACACACGTACTACA |
| | | | ATGGCAGTCATACAGAGGGAAGCAAA |
| | | | GCTGTGAGGCGGAGCAAATCCCTAAA |
| | | | AGCTGTCCCAGTTCAGATTGCAGGCT |
| | | | GCAACCCGCCTGCATGAAGTCGGAAT |
| | | | TGCTAGTAATCGCGGATCAGCATGCC |
| | | | GCGGTGAATACGTTCCCGGGCCTTGT |
| | | | ACACACCGCCCGTCACACCATGAGAG |
| | | | CCGTCAATACCCGAAGTCCGTAGCCT |
| | | | AACCGCAAGGAGGGCGCGGCCGAAGG |
| | | | TAGGGGTGGTAATTAGGGTGAAGTCG |
| | | | TAACAAGGTAGCCGTATCGGAAGGTG |
| | | | CGGCTGGATCACCTCCTTT |
| 5 | Acutalibacter | PTA-127006 | TTTAGAGAGTTTGATCCTGGCTCAGG |
| | sp. Strain A | | ACGAACGCTGGCGGCGTGCCTAACAC |
| | | | ATGCAAGTCGAACGGAGATAAGCGCT |
| | | | GATGATTTAGCTTGCTATTGATTCTT |
| | | | GTTTATCTTAGTGGCGGACGGGTGAG |
| | | | TAACGCGTGAGCAACCTGCCTTTCAG |
| | | | AGGGGGATAACGTCTTGAAAAGGACG |
| | | | CTAATACCGCATGAGATCGTAGCCCC |
| | | | ACATGGGACAGCGACCAAAGGAGCAA |
| | | | TCCGCTGAAAGATGGGCTCGCGTCCG |
| | | | ATTAGATAGTTGGCGGGGTAACGGCC |
| | | | CACCAAGTCGACGATCGGTAGCCGGA |
| | | | CTGAGAGGTTGAACGGCCACATTGGG |
| | | | ACTGAGACACGGCCCAGACTCCTACG |
| | | | GGAGGCAGCAGTGAGGGATATTGGTC |
| | | | AATGGGGGAAACCCTGAACCAGCAAC |
| | | | GCCGCGTGAGGGATGACGGCCTTCGG |
| | | | GTTGTAAACCTCTGTCCTCTGTGAAG |
| | | | ATAATGACGGTAGCAGAGGAGGAAGC |
| | | | TCCGGCTAACTACGTGCCAGCAGCCG |
| | | | CGGTAATACGTAGGGAGCAAGCGTTG |
| | | | TCCGGATTTACTGGGTGTAAAGGGTG |
| | | | CGTAGGCGGTTCGGCAAGTCAGAAGT |
| | | | GAAATCCATGGGCTTAACCCATGAAC |
| | | | TGCTTTTGAAACTGTCGAACTTGAGT |
| | | | GAAGTAGAGGTAGGCGGAATTCCCGG |
| | | | TGTAGCGGTGAAATGCGTAGAGATCG |
| | | | GGAGGAACACCAGTGGCGAAGGCGGC |
| | | | CTACTGGGCTTTAACTGACGCTGAGG |
| | | | CACGAAAGCATGGGTAGCAAACAGGA |
| | | | TTAGATACCCTGGTAGTCCATGCCGT |
| | | | AAACGATGATTACTAGGTGTGGGGGG |
| | | | TCTGACCCCCTCCGTGCCGGAGTTAA |
| | | | CACAATAAGTAATCCACCTGGGGAGT |
| | | | ACGGCCGCAAGGCTGAAACTCAAAGG |
| | | | AATTGACGGGGGCCCGCACAAGCAGT |
| | | | GGAGTATGTGGATTAATTCGAAGCAA |
| | | | CGCGAAGAACCTTACCAGGTCTTGAC |
| | | | ATCCAGCTAACGAAGTAGAGATACAT |
| | | | TAGGTGCCCTTCGGGGAAAGCTGAGA |
| | | | CAGGTGGTGCATGGTTGTCGTCAGCT |
| | | | CGTCAACGAGCGCAACCCTTACTGTT |
| | | | AGTTGCTACGCAAGAGCACTCTAGCA |
| | | | GGACCGCCGTTGACAAAACGGAGGAA |
| | | | GGTGGGGATGATGTCAAATCATCATG |
| | | | CCCCTTATGACCTGGGCCTCACACGT |
| | | | ACTACAATGGCCGTAAACAGAGGGAA |
| | | | GCAATACCGCGAGGTGGAGCAAAACC |
| | | | CTAAAAACGGTCCCAGTTCGGATTGT |
| | | | AGGCTGCAACCCGCCTGCATGAAGTT |
| | | | GGAATTGCTAGTAATCGCGGATCAGC |
| | | | ATGCCGCGGTGAATACGTTCCCGGGC |
| | | | CTTGTACACACCGCCCGTCACACCAT |
| | | | GGGAGCCGGTAATACCCGAAGTCAGT |
| | | | AGTCTAACCGCAAGGAGGGCGCTGCC |
| | | | GAAGGTAGGATTGGCGACTGGGGTGA |
| | | | AGTCGTAACAAGGTAGCCGTATCGGA |
| | | | AGGTGCGGCTGGATCACCTCCTTT |
| 6 | Anaerotruncus | PTA-127005 | CAAAGAGTTTGATCCTGGCTCAGGAC |
| | colihominis | | GAACGCTGGCGGCGCGCCTAACACAT |
| | Strain A | | GCAAGTCGAACGGAGCTTAGATTTTG |
| | | | AAGTTTTCGGATGGATGAATGTAAGC |
| | | | TTAGTGGCGGACGGGTGAGTAACACG |
| | | | TGAGCAACCTGCCTTTCAGAGGGGGA |
| | | | TAACAGCCGGAAACGGCTGCTAATAC |
| | | | CGCATGATGTTGCGGGGGCACATGCC |
| | | | CCTGCAACCAAAGGAGCAATCCGCTG |
| | | | AAAGATGGGCTCGCGTCCGATTAGCC |
| | | | AGTTGGCGGGGTAACGGCCCACCAAA |
| | | | GCGACGATCGGTAGCCGGACTGAGAG |
| | | | GTTGAACGGCCACATTGGGACTGAGA |
| | | | CACGGCCCAGACTCCTACGGGAGGCA |
| | | | GCAGTGGGGGATATTGCACAATGGGC |
| | | | GAAAGCCTGATGCAGCGACGCCGCGT |
| | | | GAGGGAAGACGGTCTTCGGATTGTAA |
| | | | ACCTCTGTCTTTGGGGAAGAAAATGA |
| | | | CGGTACCCAAAGAGGAAGCTCCGGCT |
| | | | AACTACGTGCCAGCAGCCGCGGTAAT |
| | | | ACGTAGGGAGCAAGCGTTGTCCGGAA |
| | | | TTACTGGGTGTAAAGGGAGCGTAGGC |
| | | | GGGATGGCAAGTAGAATGTTAAATCC |
| | | | ATCGGCTCAACCGGTGGCTGCGTTCT |
| | | | AAACTGCCGTTCTTGAGTGAAGTAGA |
| | | | GGCAGGCGGAATTCCTAGTGTAGCGG |
| | | | TGAAATGCGTAGATATTAGGAGGAAC |
| | | | ACCAGTGGCGAAGGCGGCCTGCTGGG |
| | | | CTTTAACTGACGCTGAGGCTCGAAAG |
| | | | CGTGGGGAGCAAACAGGATTAGATAC |
| | | | CCTGGTAGTCCACGCCGTAAACGATG |
| | | | ATTACTAGGTGTGGGGGGACTGACCC |
| | | | CTTCCGTGCCGCAGTTAACACAATAA |
| | | | GTAATCCACCTGGGGAGTACGGCCGC |
| | | | AAGGTTGAAACTCAAAGGAATTGACG |
| | | | GGGGCCCGCACAAGCAGTGGAGTATG |
| | | | TGGTTTAATTCGAAGCAACGCGAAGA |
| | | | ACCTTACCAGGTCTTGACATCGGATG |
| | | | CATAGCCTAGAGATAGGTGAAGCCCT |
| | | | TCGGGGCATCCAGACAGGTGGTGCAT |
| | | | GGTTGTCGTCAGCTCGTGTCGTGAGA |
| | | | TGTTGGGTTAAGTCCCGCAACGAGCG |
| | | | CAACCCTTATTATTAGTTGCTACGCA |
| | | | AGAGCACTCTAATGAGACTGCCGTTG |
| | | | ACAAAACGGAGGAAGGTGGGGATGAC |
| | | | GTCAAATCATCATGCCCCTTATGACC |
| | | | TGGGCTACACACGTACTACAATGGCA |
| | | | CTAAAACAGAGGGCGGCGACACCGCG |
| | | | AGGTGAAGCGAATCCCGAAAAAGTGT |
| | | | CTCAGTTCAGATTGCAGGCTGCAACC |
| | | | CGCCTGCATGAAGTCGGAATTGCTAG |
| | | | TAATCGCGGATCAGCATGCCGCGGTG |
| | | | AATACGTTCCCGGGCCTTGTACACAC |
| | | | CGCCCGTCACACCATGGGAGTCGGTA |
| | | | ACACCCGAAGCCAGTAGCCTAACCGC |
| | | | AAGGGGGGCGCTGTCGAAGGTGGGAT |
| | | | TGATGACTGGGGTGAAGTCGTAACAA |
| | | | GGTAGCCGTATCGGAAGGTGCGGCTG |
| | | | GATCACCTCCTTT |
| 7 | Subdoligranulum | PTA-127004 | AGATGCATGAAGTCCTTCGGGACATC |
| | variabile | | GAGACAGGTGGTGCATGGTTGTCGTC |
| | Strain A | | AGCTCGTGTCGTGAGATGTTGGGTTA |
| | | | AGTCCCGCAACGAGCGCAACCCTTAT |
| | | | TGCCAGTTACTACGCAAGAGGACTCT |
| | | | GGCGAGACTGCCGTTGACAAAACGGA |
| | | | GGAAGGTGGGGATGACGTCAAATCAT |
| | | | CATGCCCTTTATGACCTGGGCTACAC |
| | | | ACGTACTACAATGGCGTTTAACAAAG |
| | | | AGAAGCAAGACCGCGAGGTGGAGCAA |
| | | | AACTCAAAAACAACGTCTCAGTTCAG |
| | | | ATTGCAGGCTGCAACTCGCCTGCATG |
| | | | AAGTCGGAATTGCTAGTAATCGCGGA |
| | | | TCAGCATGCCGCGGTGAATACGTTCC |
| | | | CGGGCCTTGTACACACCGCCCGTCAC |
| | | | ACCATGAGAGCCGGGGGGACCCGAAG |
| | | | TCGGTAGTCTAACCGCAAGGAGGACG |
| | | | CCGCCGAAGGTAAAACTGGTGATTGG |
| | | | GGTGAAGTCGTAACAAGGTAGCCGTA |
| | | | TCGGAAGGTGCGGCTGGATCACCTCC |
| | | | TTT (Partial Sequence) |

Under the terms of the Budapest Treaty on the International Recognition of the Deposit of Microorganisms for the Purpose of Patent Procedure, the Faecalibacterium prausnitzii Strain A was deposited on Jun. 18, 2020, with the American Type Culture Collection (ATCC) of 10801 University Boulevard, Manassas, Va. 20110-2209 USA and was assigned ATCC Accession Number PTA-126792. Under the terms of the Budapest Treaty on the International Recognition of the Deposit of Microorganisms for the Purpose of Patent Procedure, the Fournierella massiliensis strain A was deposited on Mar. 20, 2020, with the American Type Culture Collection (ATCC) of 10801 University Boulevard, Manassas, Va. 20110-2209 USA and was assigned ATCC Accession Number PTA-126696. Under the terms of the Budapest Treaty on the International Recognition of the Deposit of Microorganisms for the Purpose of Patent Procedure, the Harryflintia acetispora strain A was deposited on Mar. 20, 2020, with the American Type Culture Collection (ATCC) of 10801 University Boulevard, Manassas, Va. 20110-2209 USA and was assigned ATCC Accession Number PTA-126694. Under the terms of the Budapest Treaty on the International Recognition of the Deposit of Microorganisms for the Purpose of Patent Procedure, the Agathobaculum sp. strain A was deposited on Apr. 11, 2019, with the American Type Culture Collection (ATCC) of 10801 University Boulevard, Manassas, Va. 20110-2209 USA and was assigned ATCC Accession Number PTA-125892. Under the terms of the Budapest Treaty on the International Recognition of the Deposit of Microorganisms for the Purpose of Patent Procedure, the Acutalibacter sp. strain A was deposited on Apr. 16, 2021, with the American Type Culture Collection (ATCC) of 10801 University Boulevard, Manassas, Va. 20110-2209 USA and was assigned ATCC Accession Number PTA-127006. Under the terms of the Budapest Treaty on the International Recognition of the Deposit of Microorganisms for the Purpose of Patent Procedure, the Anaerotruncus colihominis sp. strain A was deposited on Apr. 16, 2021, with the American Type Culture Collection (ATCC) of 10801 University Boulevard. Manassas. Va. 20110-2209 USA and was assigned ATCC Accession Number PTA-127005. Under the terms of the Budapest Treaty on the International Recognition of the Deposit of Microorganisms for the Purpose of Patent Procedure, the Subdoligranulum variabile strain A was deposited on Apr. 16, 2021, with the American Type Culture Collection (ATCC) of 10801 University Boulevard, Manassas, Va. 20110-2209 USA and was assigned ATCC Accession Number PTA-127004.

Applicant represents that the ATCC is a depository affording permanence of the deposit and ready accessibility thereto by the public if a patent is granted. All restrictions on the availability to the public of the material so deposited will be irrevocably removed upon the granting of a patent. The material will be available during the pendency of the patent application to one determined by the Commissioner to be entitled thereto under 37 CFR 1.14 and 35 U.S.C. 122. The deposited material will be maintained with all the care necessary to keep it viable and uncontaminated for a period of at least five years after the most recent request for the furnishing of a sample of the deposited plasmid, and in any case, for a period of at least thirty (30) years after the date of deposit or for the enforceable life of the patent, whichever period is longer. Applicant acknowledges its duty to replace the deposit should the depository be unable to furnish a sample when requested due to the condition of the deposit.

In some embodiments, Oscillospiraceae bacteria from which mEVs are obtained are lyophilized. In some embodiments, Oscillospiraceae bacteria from which mEVs are obtained are gamma irradiated (e.g., at 17.5 or 25 kGy). In some embodiments, Oscillospiraceae bacteria from which mEVs are obtained are UV irradiated. In some embodiments, Oscillospiraceae bacteria from which mEVs are obtained are heat inactivated (e.g., at 50° C. for two hours or at 90° C. for two hours). In some embodiments, Oscillospiraceae bacteria from which mEVs are obtained are acid treated. In some embodiments, Oscillospiraceae bacteria from which mEVs are obtained are oxygen sparged (e.g., at 0.1 vvm for two hours).

In some embodiments, the mEVs are lyophilized. In some embodiments, the mEVs are gamma irradiated (e.g., at 17.5 or 25 kGy). In some embodiments, the mEVs are UV irradiated. In some embodiments, the mEVs are heat inactivated (e.g., at 50° C. for two hours or at 90° C. for two hours). In some embodiments, the mEVs are acid treated. In some embodiments, the mEVs are oxygen sparged (e.g., at 0.1 vvm for two hours).

The phase of growth can affect the amount or properties of Oscillospiraceae bacteria and/or smEVs produced by Oscillospiraceae bacteria. For example, in the methods of smEVs preparation provided herein, smEVs can be isolated, e.g., from a culture, at the start of the log phase of growth, midway through the log phase, and/or once stationary phase growth has been reached. As another example, in the methods of pmEV preparation provided herein, pmEVs can be prepared from a culture, at the start of the log phase of growth, midway through the log phase, and/or once stationary phase growth has been reached.

Modified mEVs

In some aspects, the mEVs (such as smEVs and/or pmEVs) described herein are modified such that they comprise, are linked to, and/or are bound by a therapeutic moiety.

In some embodiments, the therapeutic moiety is a cancer-specific moiety. In some embodiments, the cancer-specific moiety has binding specificity for a cancer cell (e.g., has binding specificity for a cancer-specific antigen). In some embodiments, the cancer-specific moiety comprises an antibody or antigen binding fragment thereof. In some embodiments, the cancer-specific moiety comprises a T cell receptor or a chimeric antigen receptor (CAR). In some embodiments, the cancer-specific moiety comprises a ligand for a receptor expressed on the surface of a cancer cell or a receptor-binding fragment thereof. In some embodiments, the cancer-specific moiety is a bipartite fusion protein that has two parts: a first part that binds to and/or is linked to the bacterium and a second part that is capable of binding to a cancer cell (e.g., by having binding specificity for a cancer-specific antigen). In some embodiments, the first part is a fragment of or a full-length peptidoglycan recognition protein, such as PGRP. In some embodiments the first part has binding specificity for the mEV (e.g., by having binding specificity for a bacterial antigen). In some embodiments, the first and/or second part comprises an antibody or antigen binding fragment thereof. In some embodiments, the first and/or second part comprises a T cell receptor or a chimeric antigen receptor (CAR). In some embodiments, the first and/or second part comprises a ligand for a receptor expressed on the surface of a cancer cell or a receptor-binding fragment thereof. In certain embodiments, co-administration of the cancer-specific moiety with the mEVs (either in combination or in separate administrations) increases the targeting of the mEVs to the cancer cells.

In some embodiments, the mEVs described herein are modified such that they comprise, are linked to, and/or are bound by a magnetic and/or paramagnetic moiety (e.g., a magnetic bead) In some embodiments, the magnetic and/or paramagnetic moiety is comprised by and/or directly linked to the bacteria. In some embodiments, the magnetic and/or paramagnetic moiety is linked to and/or a part of an mEV-binding moiety that that binds to the mEV. In some embodiments, the mEV-binding moiety is a fragment of or a full-length peptidoglycan recognition protein, such as PGRP. In some embodiments the mEV-binding moiety has binding specificity for the mEV (e.g., by having binding specificity for a bacterial antigen). In some embodiments, the mEV-binding moiety comprises an antibody or antigen binding fragment thereof. In some embodiments, the mEV-binding moiety comprises a T cell receptor or a chimeric antigen receptor (CAR). In some embodiments, the mEV-binding moiety comprises a ligand for a receptor expressed on the surface of a cancer cell or a receptor-binding fragment thereof. In certain embodiments, co-administration of the magnetic and/or paramagnetic moiety with the mEVs (either together or in separate administrations) can be used to increase the targeting of the mEVs (e.g., to cancer cells and/or a part of a subject where cancer cells are present.

Production of Processed Microbial Extracellular Vesicles (pmEVs)

In certain aspects, the pmEVs described herein can be prepared using any method known in the art.

In some embodiments, the pmEVs are prepared without a pmEV purification step. For example, in some embodiments, Oscillospiraceae bacteria from which the pmEVs described herein are released are killed using a method that leaves the Oscillospiraceae bacterial pmEVs intact, and the resulting Oscillospiraceae bacterial components, including the pmEVs, are used in the methods and compositions described herein. In some embodiments, the Oscillospiraceae bacteria are killed using an antibiotic (e.g., using an antibiotic described herein). In some embodiments, the Oscillospiraceae bacteria are killed using UV irradiation.

In some embodiments, the pmEVs described herein are purified from one or more other Oscillospiraceae bacterial components. Methods for purifying pmEVs from Oscillospiraceae bacteria (and optionally, other bacterial components) are known in the art. In some embodiments, pmEVs are prepared from Oscillospiraceae bacterial cultures using methods described in Thein, et al. (J. Proteome Res. 9(12):6135-6147 (2010)) or Sandrini, et al. (Bio-protocol 4(21): e1287 (2014)), each of which is hereby incorporated by reference in its entirety. In some embodiments, the bacteria are cultured to high optical density and then centrifuged to pellet bacteria (e.g., at 10,000-15,000×g for 10-15 min at room temperature or 4° C.) In some embodiments, the supernatants are discarded and cell pellets am frozen at −80° C. In some embodiments, cell pellets are thawed on ice and resuspended in 100 mM Tris-HCl, pH 7.5 supplemented with 1 mg/mL DNase I. In some embodiments, cells are lysed using an Emulsifier C-3 (Avestin, Inc.) under conditions recommended by the manufacturer. In some embodiments, debris and unlysed cells are pelleted by centrifugation at 10,000×g for 15 min at 4° C. In some embodiments, supernatants are then centrifuged at 120,000×g for 1 hour at 4° C. In some embodiments, pellets are resuspended in ice-cold 100 mM sodium carbonate. pH 11, incubated with agitation for 1 hr at 4° C. and then centrifuged at 120,000×g for 1 hour at 4° C. In some embodiments, pellets are resuspended in 100 mM Tris-HCl, pH 7.5, re-centrifuged at 120,000×g for 20 min at 4° C., and then resuspended in 0.1 M Tris-HCl, pH 7.5 or in PBS. In some embodiments, samples are stored at −2(0′C.

In certain aspects, pmEVs are obtained by methods adapted from Sandrini et al, 2014. In some embodiments, Oscillospiraceae bacterial cultures are centrifuged at 10,000-15,500×g for 10-15 min at room temp or at 4° C. In some embodiments, cell pellets are frozen at −80° C. and supernatants are discarded. In some embodiments, cell pellets are thawed on ice and resuspended in 10 mM Tris-HCl, pH 8.0, 1 mM EDTA supplemented with 0.1 mg/mL lysozyme. In some embodiments, samples are incubated with mixing at room temp or at 37° C. for 30 min. In some embodiments, samples are re-frozen at −80° C. and thawed again on ice. In some embodiments, DNase I is added to a final concentration of 1.6 mg/mL and MgCl2 to a final concentration of 100 mM. In some embodiments, samples are sonicated using a QSonica Q500 sonicator with 7 cycles of 30 sec on and 30 sec off. In some embodiments, debris and unlysed cells are pelleted by centrifugation at 10,000×g for 15 min. at 4° C. In some embodiments, supernatants are then centrifuged at 110,000×g for 15 min at 4° C. In some embodiments, pellets are resuspended in 10 mM Tris-HCl, pH 8.0, 2% Triton X-100 and incubated 30-60 min with mixing at room temperature. In some embodiments, samples are centrifuged at 110,000×g for 15 min at 4° C. In some embodiments, pellets are resuspended in PBS and stored at −20° C.

In certain aspects, a method of forming (e.g., preparing) isolated Oscillospiraceae bacterial pmEVs, described herein, comprises the steps of: (a) centrifuging a Oscillospiraceae bacterial culture, thereby forming a first pellet and a first supernatant, wherein the first pellet comprises cells; (b) discarding the first supernatant; (c) resuspending the first pellet in a solution; (d) lysing the cells; (e) centrifuging the lysed cells, thereby forming a second pellet and a second supernatant; (f) discarding the second pellet and centrifuging the second supernatant, thereby forming a third pellet and a third supernatant; (g) discarding the third supernatant and resuspending the third pellet in a second solution, thereby forming the isolated Oscillospiraceae bacterial pmEVs.

In some embodiments, the method further comprises the steps of: (h) centrifuging the solution of step (g), thereby forming a fourth pellet and a fourth supernatant; (i) discarding the fourth supernatant and resuspending the fourth pellet in a third solution. In some embodiments, the method further comprises the steps of: (j) centrifuging the solution of step (i), thereby forming a fifth pellet and a fifth supernatant; and (k) discarding the fifth supernatant and resuspending the fifth pellet in a fourth solution.

In some embodiments, the centrifugation of step (a) is at 10,000×g. In some embodiments the centrifugation of step (a) is for 10-15 minutes. In some embodiments, the centrifugation of step (a) is at 4° C. or room temperature. In some embodiments, step (b) further comprises freezing the first pellet at −80° C. In some embodiments, the solution in step (c) is 100 mM Tris-HCl, pH 7.5 supplemented with 1 mg/ml DNaseI. In some embodiments, the solution in step (c) is 10 mM Tris-HCl, pH 8.0, 1 mM EDTA, supplemented with 0.1 mg/ml lysozyme. In some embodiments, step (c) further comprises incubating for 30 minutes at 37° C. or room temperature. In some embodiments, step (c) further comprises freezing the first pellet at −80° C. In some embodiments, step (c) further comprises adding DNase I to a final concentration of 1.6 mg/ml. In some embodiments, step (c) further comprises adding MgCl₂ to a final concentration of 100 mM. In some embodiments, the cells are lysed in step (d) via homogenization. In some embodiments, the cells are lysed in step (d) via emulsiflex C3. In some embodiments, the cells are lysed in step (d) via sonication. In some embodiments, the cells are sonicated in 7 cycles, wherein each cycle comprises 30 seconds of sonication and 30 seconds without sonication. In some embodiments, the centrifugation of step (e) is at 10,000×g. In some embodiments, the centrifugation of step (e) is for 15 minutes. In some embodiments, the centrifugation of step (e) is at 4° C. or room temperature.

In some embodiments, the centrifugation of step (f) is at 120,000×g. In some embodiments, the centrifugation of step (f) is at 110,000×g. In some embodiments, the centrifugation of step (f) is for 1 hour. In some embodiments, the centrifugation of step (f) is for 15 minutes. In some embodiments, the centrifugation of step (f) is at 4° C. or room temperature. In some embodiments, the second solution in step (g) is 100 mM sodium carbonate, pH 11. In some embodiments, the second solution in step (g) is 10 mM Tris-HCl pH 8.0, 2% triton X-100. In some embodiments, step (g) further comprises incubating the solution for 1 hour at 4° C. In some embodiments, step (g) further comprises incubating the solution for 30-60 minutes at room temperature. In some embodiments, the centrifugation of step (h) is at 120,000×g. In some embodiments, the centrifugation of step (h) is at 110,000×g. In some embodiments, the centrifugation of step (h) is for 1 hour. In some embodiments, the centrifugation of step (h) is for 15 minutes. In some embodiments, the centrifugation of step (h) is at 4° C. or room temperature. In some embodiments, the third solution in step (i) is 100 mM Tris-HCl, pH 7.5. In some embodiments, the third solution in step (i) is PBS. In some embodiments, the centrifugation of step (j) is at 120,000×g. In some embodiments, the centrifugation of step (j) is for 20 minutes. In some embodiments, the centrifugation of step (j) is at 4° C. or room temperature. In some embodiments, the fourth solution in step (k) is 100 mM Tris-HCl, pH 7.5 or PBS.

pmEVs obtained by methods provided herein may be further purified by size based column chromatography, by affinity chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in 35% Optiprep in PBS. In some embodiments, if filtration was used to concentrate the filtered supernatant, the concentrate is diluted using 60% Optiprep to a final concentration of 35% Optiprep. Samples are applied to a 35-60% discontinuous sucrose gradient and centrifuged at 200,000×g for 3-24 hours at 4° C.

In some embodiments, to confirm sterility and isolation of the pmEV preparations, pmEVs are serially diluted onto agar medium used for routine culture of the bacteria being tested, and incubated using routine conditions. Non-sterile preparations are passed through a 0.22 μm filter to exclude intact cells. To further increase purity, isolated pmEVs may be DNase or proteinase K treated.

In some embodiments, the sterility of the pmEV preparations can be confirmed by plating a portion of the pmEVs onto agar medium used for standard culture of the bacteria used in the generation of the pmEVs and incubating using standard conditions.

In some embodiments select pmEVs are isolated and enriched by chromatography and binding surface moieties on pmEVs. In other embodiments, select pmEVs are isolated and/or enriched by fluorescent cell sorting by methods using affinity reagents, chemical dyes, recombinant proteins or other methods known to one skilled in the art.

The pmEVs can be analyzed. e.g., as described in Jeppesen, et al. Cell 177:428 (2019).

In some embodiments, pmEVs are lyophilized. In some embodiments, pmEVs are gamma irradiated (e.g., at 17.5 or 25 kGy). In some embodiments, pmEVs are UV irradiated. In some embodiments, pmEVs are heat inactivated (e.g., at 50° C. for two hours or at 90° C. for two hours). In some embodiments, pmEVs are acid treated. In some embodiments, pmEVs are oxygen sparged (e.g., at 0.1 vvm for two hours).

The phase of growth can affect the amount or properties of bacteria. In the methods of pmEV preparation provided herein, pmEVs can be isolated, e.g., from a culture, at the start of the log phase of growth, midway through the log phase, and/or once stationary phase growth has been reached.

Production of Secreted Microbial Extracellular Vesicles (smEVs)

In certain aspects, the smEVs described herein can be prepared using any method known in the art.

In some embodiments, the smEVs are prepared without an smEV purification step. For example, in some embodiments, bacteria described herein are killed using a method that leaves the smEVs intact and the resulting Oscillospiraceae bacterial components, including the smEVs, are used in the methods and compositions described herein. In some embodiments, the Oscillospiraceae bacteria are killed using an antibiotic (e.g., using an antibiotic described herein). In some embodiments, the Oscillospiraceae bacteria are killed using UV irradiation. In some embodiments, the Oscillospiraceae bacteria are heat-killed.

In some embodiments, the smEVs described herein are purified from one or more other Oscillospiraceae bacterial components. Methods for purifying smEVs from bacteria are known in the art. In some embodiments, smEVs are prepared from Oscillospiraceae bacterial cultures using methods described in S. Bin Park, et al. PLoS ONE. 6(3):e17629 (2011) or G. Norheim, et al. PLoS ONE. 10(9): e0134353 (2015) or Jeppesen, et al. Cell 177:428 (2019), each of which is hereby incorporated by reference in its entirety. In some embodiments, the Oscillospiraceae bacteria are cultured to high optical density and then centrifuged to pellet Oscillospiraceae bacteria (e.g., at 10,000×g for 30 min at 4° C., at 15,500×g for 15 min at 4° C.). In some embodiments, the culture supernatants are then passed through filters to exclude intact bacterial cells (e.g., a 0.22 μm filter). In some embodiments, the supernatants are then subjected to tangential flow filtration, during which the supernatant is concentrated, species smaller than 100 kDa are removed, and the media is partially exchanged with PBS. In some embodiments, filtered supernatants are centrifuged to pellet bacterial smEVs (e.g., at 100,000-150,000×g for 1-3 hours at 4° C., at 200,000×g for 1-3 hours at 4° C.). In some embodiments, the smEVs are further purified by resuspending the resulting smEV pellets (e.g., in PBS), and applying the resuspended smEVs to an Optiprep (iodixanol) gradient or gradient (e.g., a 30-60% discontinuous gradient, a 045% discontinuous gradient), followed by centrifugation (e.g., at 200,000×g for 4-20 hours at 4° C.). smEV bands can be collected, diluted with PBS, and centrifuged to pellet the smEVs (e.g., at 150,000×g for 3 hours at 4° C., at 200,000×g for 1 hour at 4° C.). The purified smEVs can be stored, for example, at −80° C. or −20° C. until use. In some embodiments, the smEVs are further purified by treatment with DNase and/or proteinase K.

For example, in some embodiments, cultures of Oscillospiraceae bacteria can be centrifuged at 11,000×g for 20-40 min at 4° C. to pellet bacteria. Culture supernatants may be passed through a 0.22 μm filter to exclude intact bacterial cells. Filtered supernatants may then be concentrated using methods that may include, but are not limited to, ammonium sulfate precipitation, ultracentrifugation, or filtration. For example, for ammonium sulfate precipitation, 1.5-3 M ammonium sulfate can be added to filtered supernatant slowly, while stirring at 4° C. Precipitations can be incubated at 4° C. for 8-48 hours and then centrifuged at 11,000×g for 20-40 min at 4° C. The resulting pellets contain bacteria smEVs and other debris. Using ultracentrifugation, filtered supernatants can be centrifuged at 100,000-200,000×g for 1-16 hours at 4° C. The pellet of this centrifugation contains bacteria smEVs and other debris such as large protein complexes. In some embodiments, using a filtration technique, such as through the use of an Amicon Ultra spin filter or by tangential flow filtration, supernatants can be filtered so as to retain species of molecular weight >50 or 100 kDa.

Alternatively, smEVs can be obtained from Oscillospiraceae bacteria cultures continuously during growth, or at selected time points during growth, for example, by connecting a bioreactor to an alternating tangential flow (ATF) system (e.g., XCell ATF from Repligen). The ATF system retains intact cells (>0.22 μm) in the bioreactor, and allows smaller components (e.g., smEVs, free proteins) to pass through a filter for collection. For example, the system may be configured so that the <0.22 μm filtrate is then passed through a second filter of 100 kDa, allowing species such as smEVs between 0.22 μm and 100 kDa to be collected, and species smaller than 100 kDa to be pumped back into the bioreactor Alternatively, the system may be configured to allow for medium in the bioreactor to be replenished and/or modified during growth of the culture. smEVs collected by this method may be further purified and/or concentrated by ultracentrifugation or filtration as described above for filtered supernatants.

smEVs obtained by methods provided herein may be further purified by size-based column chromatography, by affinity chromatography, by ion-exchange chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amnicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in PBS and 3 volumes of 60% Optiprep are added to the sample. In some embodiments, if filtration was used to concentrate the filtered supernatant, the concentrate is diluted using 60% Optiprep to a final concentration of 35% Optiprep. Samples are applied to a 0-45% discontinuous Optiprep gradient and centrifuged at 200,000×g for 3-24 hours at 4° C., e.g., 4-24 hours at 4° C.

In some embodiments, to confirm sterility and isolation of the smEV preparations, smEVs are serially diluted onto agar medium used for routine culture of the bacteria being tested, and incubated using routine conditions. Non-sterile preparations are passed through a 0.22 μm filter to exclude intact cells. To further increase purity, isolated smEVs may be DNase or proteinase K treated.

In some embodiments, for preparation of smEVs used for in vivo injections, purified smEVs are processed as described previously (G. Norheim, et al. PLoS ONE. 10(9): e0134353 (2015)). Briefly, after sucrose gradient centrifugation, bands containing smEVs are resuspended to a final concentration of 50 μg/mL in a solution containing 3% sucrose or other solution suitable for in vivo injection known to one skilled in the art. This solution may also contain adjuvant, for example aluminum hydroxide at a concentration of 0-0.5% (w/v). In some embodiments, for preparation of smEVs used for in vivo injections, smEVs in PBS are sterile-filtered to <0.22 μm.

In certain embodiments, to make samples compatible with further testing (e.g., to remove sucrose prior to TEM imaging or in vitro assays), samples are buffer exchanged into PBS or 30 mM Tris, pH 8.0 using filtration (e.g., Amicon Ultra columns), dialysis, or ultracentrifugation (200,000×g, ≥3 hours, 4° C.) and resuspension.

In some embodiments, the sterility of the smEV preparations can be confirmed by plating a portion of the smEVs onto agar medium used for standard culture of the bacteria used in the generation of the smEVs and incubating using standard conditions.

In some embodiments, select smEVs are isolated and enriched by chromatography and binding surface moieties on smEVs. In other embodiments, select smEVs are isolated and/or enriched by fluorescent cell sorting by methods using affinity reagents, chemical dyes, recombinant proteins or other methods known to one skilled in the art.

The smEVs can be analyzed, e.g., as described in Jeppesen, et al. Cell 177.428 (2019).

In some embodiments, smEVs are lyophilized. In some embodiments, smEVs are gamma irradiated (e.g., at 17.5 or 25 kGy) In some embodiments, smEVs are UV irradiated. In some embodiments, smEVs are heat inactivated (e.g., at 50° C. for two hours or at 90° C. for two hours). In some embodiments, smEVs s are acid treated. In some embodiments, smEVs are oxygen sparged (e.g., at 0.1 vvm for two hours).

The phase of growth can affect the amount or properties of Oscillospiraceae bacteria and/or smEVs produced by Oscillospiraceae bacteria. For example, in the methods of smEV preparation provided herein, smEVs can be isolated, e.g., from a culture, at the start of the log phase of growth, midway through the log phase, and/or once stationary phase growth has been reached.

The growth environment (e.g., culture conditions) can affect the amount of smEVs produced by Oscillospiraceae bacteria. For example, the yield of smEVs can be increased by an smEV inducer, as provided in Table 3.

TABLE 3
Culture Techniques to Increase smEV Production

| smEV inducement | smEV inducer | Acts on |
|---|---|---|
| Temperature | | |
| | Heat | stress response |
| | RT to 37° C. | simulates infection |
| | temp change | |
| | 37 to 40° C. | febrile infection |
| | temp change | |
| ROS | | |
| | Plumbagin | oxidative stress response |
| | Cumene hydroperoxide | oxidative stress response |
| | Hydrogen Peroxide | oxidative stress response |
| Antibiotics | | |
| | Ciprofloxacin | bacterial SOS response |
| | Gentamycin | protein synthesis |
| | Polymyxin B | outer membrane |
| | D-cylcloserine | cell wail |
| Osmolyte | | |
| | NaCl | osmotic stress |
| Metal | | |
| Ion Stress | | |
| | Iron Chelation | iron levels |
| | EDTA | removes divalent cations |
| | Low Hemin | iron levels |
| Media additives | | |
| or removal | | |
| | Lactate | growth |
| | Ammo acid deprivation | stress |
| | Hexadecane | stress |
| | Glucose | growth |
| | Sodium bicarbonate | ToxT induction |
| | PQS | vesiculator |
| | | (from bacteria) |
| | Diamines + DFMO | membrane anchoring |
| | | (negativicutes only) |
| | High nutrients | enhanced growth |
| | Low nutrients | |
| Other mechanisms | | |
| | Oxygen | oxygen stress in anaerobe |
| | No Cysteine | oxygen stress in anaerobe |
| | Inducing biofilm | |
| | or floculation | |
| | Diauxic Growth | |
| | Phage | |
| | Urea | |

In the methods of smEVs preparation provided herein, the method can optionally include exposing a culture of Oscillospiraceae bacteria to an smEV inducer prior to isolating smEVs from the bacterial culture. The culture of Oscillospiraceae bacteria can be exposed to an smEV inducer at the start of the log phase of growth, midway through the log phase, and/or once stationary phase growth has been reached.

### Pharmaceutical Compositions

In certain embodiments, provided herein are pharmaceutical compositions (e.g., bacterial formulations or bacterial compositions) comprising mEVs (such as smEVs and/or pmEVs) (e.g., an mEV composition (e.g., an smEV composition or a pmEV composition)) from Oscillospiraceae. In some embodiments, the mEV composition comprises mEVs (such as smEVs and/or pmEVs) and/or a combination of mEVs (such as smEVs and/or pmEVs) described herein and a pharmaceutically acceptable carrier. In some embodiments, the smEV composition comprises smEVs and/or a combination of smEVs described herein and a pharmaceutically acceptable carrier. In some embodiments, the pmEV composition comprises pmEVs and/or a combination of pmEVs described herein and a pharmaceutically acceptable carrier.

In some embodiments, the pharmaceutical compositions comprise mEVs (such as smEVs and/or pmEVs) substantially or entirely free of whole bacteria (e.g., live bacteria, killed bacteria, attenuated bacteria). In some embodiments, the pharmaceutical compositions comprise both mEVs and whole bacteria (e.g., live bacteria, killed bacteria, attenuated bacteria). In some embodiments, the pharmaceutical compositions comprise mEVs from one or more (e.g., 1, 2, 3, 4, 5, 6, 7, 8, 9, 10 or more) of Oscillospiraceae bacteria strains. In some embodiments, the pharmaceutical composition comprises lyophilized mEVs (such as smEVs and/or pmEVs). In some embodiments, the pharmaceutical composition comprises gamma irradiated mEVs (such as smEVs and/or pmEVs). The mEVs (such as smEVs and/or pmEVs) can be gamma irradiated after the mEVs are isolated (e.g., prepared). In some embodiments, Oscillospiraceae strain is Faecalibacterium prausnitzii (e.g., Faecalibacterium prausnitzii strain A), Fournierella massiliensis (e.g., Fournierella massiliensis strain A), Harryflintia acetispora (e.g., Harryflintia acetispora strain A), Agathobaculum sp. (e.g., Agathobaculum sp. strain A). Acutalibacter sp. (e.g., Acutalibacter sp. strain A). Anaerotruncus colihominis (Anaerotruncus colihominis strain A), or Subdoligranulum variable (e.g., Subdoligranulum variabile strain A).

In some embodiments, to quantify the numbers of mEVs (such as smEVs and/or pmEVs) and/or bacteria present in a bacterial sample, electron microscopy (e.g., EM of ultrathin frozen sections) can be used to visualize the mEVs (such as smEVs and/or pmEVs) and/or bacteria and count their relative numbers. Alternatively, nanoparticle tracking analysis (NTA), Coulter counting, or dynamic light scattering (DLS) or a combination of these techniques can be used. NTA and the Coulter counter count particles and show their sizes DLS gives the size distribution of particles, but not the concentration. Bacteria frequently have diameters of 1-2 μm (microns). The full range is 0.2-20 μm. Combined results from Coulter counting and NTA can reveal the numbers of bacteria and/or mEVs (such as smEVs and/or pmEVs) in a given sample Coulter counting reveals the numbers of particles with diameters of 0.7-10 μm. For most bacterial and/or mEV (such as smEV and/or pmEV) samples, the Coulter counter alone can reveal the number of bacteria and/or mEVs (such as smEVs and/or pmEVs) in a sample. pmEVs are 20-600 nm in diameter. For NTA, a Nanosight instrument can be obtained from Malvern Pananlytical. For example, the NS300 can visualize and measure particles in suspension in the size range 10-2000 nm. NTA allows for counting of the numbers of particles that are, for example, 50-1000 nm in diameter. DLS reveals the distribution of particles of different diameters within an approximate range of 1 nm-3 μm.

mEVs can be characterized by analytical methods known in the art (e.g., Jeppesen, et al. Cell 177:428 (2019)).

In some embodiments, the mEVs may be quantified based on particle count. For example, particle count of an mEV preparation can be measured using NTA.

In some embodiments, the mEVs may be quantified based on the amount of protein, lipid, or carbohydrate. For example, total protein content of an mEV preparation can be measured using the Bradford assay or the BCA assay.

In some embodiments, the mEVs are isolated away from one or more other bacterial components of the source bacteria. In some embodiments, the pharmaceutical composition further comprises other bacterial components.

In certain embodiments, the mEV preparation obtained from the source bacteria may be fractionated into subpopulations based on the physical properties (e.g., sized, density, protein content, binding affinity) of the subpopulations. One or more of the mEV subpopulations can then be incorporated into the pharmaceutical compositions of the invention.

In some embodiments, the pharmaceutical composition comprises at least 1 Oscillospiraceae bacterium for every 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷ 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹² Oscillospiraceae mEV particles.

In some embodiments, the bacterial and/or pharmaceutical composition comprises about 1 Oscillospiraceae bacterium for every 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹² Oscillospiraceae mEV particles.

In some embodiments, the bacterial and/or pharmaceutical composition comprises no more than 1 Oscillospiraceae bacterium for every 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴. 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10₈, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹² Oscillospiraceae mEV particles.

In some embodiments, the bacterial and/or pharmaceutical composition comprises at least 1 Oscillospiraceae mEV particle for every 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹² Oscillospiraceae bacteria.

In some embodiments, the bacterial and/or pharmaceutical composition comprises about 1 Oscillospiraceae mEV particle for every 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7 8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴. 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹² Oscillospiraceae bacteria.

In some embodiments, the bacterial and/or pharmaceutical composition comprises no mom than 1 Oscillospiraceae mEV particle for every 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10³, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹¹ Oscillospiraceae bacteria.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total particles in the pharmaceutical composition are Oscillospiraceae mEVs.

In some embodiments, no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of total the particles in the pharmaceutical composition are Oscillospiraceae mEVs.

In some embodiments, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total particles in the pharmaceutical composition are Oscillospiraceae mEVs.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total protein in the pharmaceutical composition is Oscillospiraceae mEV protein.

In some embodiments, no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total protein in the pharmaceutical composition is Oscillospiraceae mEV protein.

In some embodiments, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total protein in the pharmaceutical composition is Oscillospiraceae mEV protein.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Oscillospiraceae mEV lipids.

In some embodiments, no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Oscillospiraceae mEV lipids.

In some embodiments, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 800, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Oscillospiraceae mEV lipids.

In certain aspects, provided herein are pharmaceutical compositions comprising mEVs (such as smEVs and/or pmEVs) useful for the treatment and/or prevention of a disease or a health disorder (e.g., adverse health disorders) (e.g., a cancer, an autoimmune disease, an inflammatory disease, a dysbiosis, or a metabolic disease), as well as methods of making and/or identifying such mEVs, and methods of using such pharmaceutical compositions (e.g., for the treatment and/or prevention of a disease or a health disorder (e.g., adverse health disorders) (e.g., a cancer, an autoimmune disease, an inflammatory disease, a dysbiosis, or a metabolic disease), either alone or in combination with other therapeutics). In some embodiments, the pharmaceutical compositions comprise both mEVs (such as smEVs and/or pmEVs), and whole bacteria (e.g., live bacteria, killed bacteria, attenuated bacteria). In some embodiments, the pharmaceutical compositions comprise mEVs (such as smEVs and/or pmEVs) in the absence of bacteria. In some embodiments, the pharmaceutical compositions comprise mEVs (such as smEVs and/or pmEVs) and/or bacteria from a Oscillospiraceae bacteria strain. In some embodiments, Oscillospiraceae strain is Faecalibacterium prausnitzii (e.g., Faecalibacterium prausnitzii strain A), Fournierella massiliensis (e.g., Fournierella massiliensis strain A), Harryflintia acetispora (e.g., Harryflintia acetispora strain A), Agathobaculum sp. (e.g., Agathobaculum sp. strain A), Acutalibacter sp. (e.g., Acutalibacter sp. strain A), Anaerotruncus colihominis (Anaerotruncus colihominis strain A), or Subdoligranulum variabile (e.g., Subdoligranulum variable strain A).

In certain aspects, provided are pharmaceutical compositions for administration to a subject (e.g., human subject). In some embodiments, the pharmaceutical compositions are combined with additional active and/or inactive materials in order to produce a final product, which may be in single dosage unit or in a multi-dose format. In some embodiments, the pharmaceutical composition is combined with an adjuvant such as an immuno-adjuvant (e.g., a STING agonist, a TLR agonist, or a NOD agonist).

In some embodiments, the pharmaceutical composition comprises at least one carbohydrate.

In some embodiments, the pharmaceutical composition comprises at least one lipid. In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20-1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0).

In some embodiments, the pharmaceutical composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the pharmaceutical composition comprises at least one supplemental vitamin. The at least one vitamin can be fat-soluble or water soluble vitamins. Suitable vitamins include but are not limited to vitamin C, vitamin A, vitamin F, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the pharmaceutical composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments, the excipient is a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the pharmaceutical composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the pharmaceutical composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the pharmaceutical composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isomorphous silicate, and microcrystalline cellulose as high HLB emulsifier surfactants.

In some embodiments, the pharmaceutical composition comprises a disintegrant as an excipient. In some embodiments the disintegrant is a non-effervescent disintegrant Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the pharmaceutical composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments, the pharmaceutical composition is a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

### Dose Forms

A pharmaceutical composition comprising mEVs (such as smEVs and/or pmEVs) from Oscillospiraceae can be formulated as a solid dose form, e.g., for oral administration. The solid dose form can comprise one or more excipients, e.g., pharmaceutically acceptable excipients. The mEVs in the solid dose form can be isolated mEVs. Optionally, the mEVs in the solid dose form can be lyophilized. Optionally, the mEVs in the solid dose form are gamma irradiated. The solid dose form can comprise a tablet, a minitablet, a capsule, a pill, or a powder; or a combination of these forms (e.g., minitablets comprised in a capsule).

The solid dose form can comprise a tablet (e.g., >4 mm).

The solid dose form can comprise a mini tablet (e.g., 1-4 mm sized minitablet, e.g., a 2 mm minitablet or a 3 mm minitablet).

The solid dose form can comprise a capsule, e.g., a size 00, size 0, size 1, size 2, size 3, size 4, or size 5 capsule; e.g., a size 0 capsule.

The solid dose form can comprise a coating. The solid dose form can comprise a single layer coating, e.g., enteric coating. e.g., a Eudragit-based coating, e.g., EUDRAGIT L30 D-55, triethylcitrate, and talc. The solid dose form can comprise two layers of coating. For example, an inner coating can comprise, e.g., EUDRAGIT L30 D-55, triethylcitrate, talc, citric acid anhydrous, and sodium hydroxide, and an outer coating can comprise. e.g., EUDRAGIT L30 D-55, triethylcitrate, and talc. EUDRAGIT is the brand name for a diverse range of polymethacrylate-based copolymers. It includes anionic, cationic, and neutral copolymers based on methacrylic acid and methacrylic/acrylic esters or their derivatives. Eudragits are amorphous polymers having glass transition temperatures between 9 to >150° C. Eudragits are non-biodegradable, nonabsorbable, and nontoxic. Anionic Eudragit L dissolves at pH>6 and is used for enteric coating, while Eudragit S, soluble at pH>7 is used for colon targeting. Eudragit RL and RS, having quaternary ammonium groups, are water insoluble, but swellable/permeable polymers which are suitable for the sustained release film coating applications. Cationic Eudragit E, insoluble at pH≥5, can prevent drug release in saliva.

The solid dose form (e.g., a capsule) can comprise a single layer coating, e.g., a non-enteric coating such as HPMC (hydroxyl propyl methyl cellulose) or gelatin.

A pharmaceutical composition comprising mEVs (such as smEVs and/or pmEVs) from Oscillospiraceae can be formulated as a suspension, e.g., for oral administration or for injection. Administration by injection includes intravenous (IV), intramuscular (IM), intratumoral (IT) and subcutaneous (SC) administration. For a suspension, mEVs can be in a buffer, e.g., a pharmaceutically acceptable buffer, e.g., saline or PBS. The suspension can comprise one or more excipients, e.g., pharmaceutically acceptable excipients. The suspension can comprise, e.g., sucrose or glucose. The mEVs in the suspension can be isolated mEVs. Optionally, the mEVs in the suspension can be lyophilized. Optionally, the mEVs in the suspension can be gamma irradiated.

### Dosage

In some embodiments, the dose of mEVs from Oscillospiraceae bacteria is about 1×10¹¹ to about 1×10¹⁴ particles (e.g., wherein particle count is determined by NTA (nanoparticle tracking analysis)).

As a further example, mEVs from Oscillospiraceae bacteria can be administered at doses e.g., of about 1×10⁷ to about 1×10¹⁵ particles, e.g., as measured by NTA. In some embodiments, the dose of mEVs is about 1×10⁵ to about 7×10¹³ particles (e.g., wherein particle count is determined by NTA (nanoparticle tracking analysis)). In some embodiments, the dose of mEVs from Oscillospiraceae massiliensis bacteria is about 1×10¹⁰ to about 7×10¹³ particles (e.g., wherein particle count is determined by NTA (nanoparticle tracking analysis)).

As another example, mEVs from Oscillospiraceae bacteria can be administered at doses e.g., of about 5 mg to about 900 mg total protein, e.g., as measured by Bradford assay. As another example, mEVs from Oscillospiraceae massiliensis bacteria can be administered at doses e.g., of about 5 mg to about 900 mg total protein, e.g., as measured by BCA assay.

mEVs from Oscillospiraceae can be administered at doses e.g., of about 1×10⁷ to about 1×10¹⁵ particles, e.g., as measured by NTA. In some embodiments, the dose of mEVs is about 1×10⁵ to about 7×10¹³ particles (e.g., wherein particle count is determined by NTA (nanoparticle tracking analysis)). In some embodiments, the dose of mEVs from bacteria is about 1×10¹⁰ to about 7×10¹³ particles (e.g., wherein particle count is determined by NTA (nanoparticle tracking analysis)). In some embodiments, the dose of mEVs from bacteria is about 1×10¹¹ to about 1×10¹⁴ particles (e.g., wherein particle count is determined by NTA (nanoparticle tracking analysis)).

As another example, mEVs from Oscillospiraceae massiliensis can be administered at doses e.g., of about 5 mg to about 900 mg total protein, e.g., as measured by Bradford assay. As another example, mEVs from bacteria can be administered at doses e.g., of about 5 mg to about 900 mg total protein, e.g., as measured by BCA assay.

For oral administration to a human subject, the dose of mEVs (such as smEVs and/or pmEVs) from Oscillospiraceae can be, e.g., about 2×10⁶-about 2×10¹⁶ particles. The dose can be, e.g., about 1×10⁷-about 1×10¹⁵, about 1×10⁸-about 1×10¹⁴, about 1×10⁹-about 1×10¹³, about 1×10¹⁰-about 1×10¹⁴, or about 1×10⁸-about 1×10¹² particles. The dose can be, e.g., about 2×10⁶, about 2×10⁷, about 2×10⁸, about 2×10⁹, about 1×10¹⁰, about 2×10¹⁰, about 2×10¹¹, about 2×10¹², about 2×10¹³, about 2×10¹⁴, or about 1×10¹⁵ particles. The dose can be, e.g., about 2×10¹⁴ particles. The dose can be, e.g., about 2×10¹² particles. The dose can be, e.g., about 2×10¹⁰ particles. The dose can be, e.g., about 1×10¹⁰ particles. Particle count can be determined, e.g., by NTA.

For oral administration to a human subject, the dose of mEVs (such as smEVs and/or pmEVs) can be, e.g., based on total protein. The dose can be, e.g., about 5 mg to about 900 mg total protein. The dose can be, e.g., about 20 mg to about 800 mg, about 50 mg to about 700 mg, about 75 mg to about 600 mg, about 100 mg to about 500 mg, about 250 mg to about 750 mg, or about 200 mg to about 500 mg total protein. The dose can be, e.g., about 10 mg, about 25 mg, about 50 mg, about 75 mg, about 100 mg, about 150 mg, about 200 mg, about 250 mg, about 300 mg, about 400 mg, about 500 mg, about 600 mg, or about 750 mg total protein. Total protein can be determined, e.g., by Bradford assay or BCA assay.

For administration by injection (e.g., intravenous administration) to a human subject, the dose of mEVs (such as smEVs and/or pmEVs) can be, e.g., about 1×10⁶-about 1×10¹⁶ particles. The dose can be, e.g., about 1×10⁷-about 1×10¹⁵, about 1×10⁸-about 1×10¹⁴, about 1×10⁹-about 1×10¹³, about 1×10¹⁰-about 1×10¹⁴, or about 1×10⁸-about 1×10¹² particles. The dose can be, e.g., about 2×10⁶, about 2×10⁷, about 2×10⁸, about 2×10⁹, about 1×10¹⁰, about 2×10¹⁰, about 2×10¹¹, about 2×10¹², about 2×10¹³, about 2×10¹⁴, or about 1×10¹⁵ particles. The dose can be, e.g., about 1×10¹⁵ particles. The dose can be, e.g., about 2×10¹⁵ particles. The dose can be, e.g., about 2×10¹³ particles. The dose can be, e.g., about 1×10¹¹ to about 1×10¹⁴ particles. The dose can be, e.g., about 1×10¹¹ particles. The dose can be, e.g., about 1×10¹² particles. The dose can be, e.g., about 1×10¹³ particles. The dose can be, e.g., about 1×10¹⁴ particles. Particle count can be determined, e.g., by NTA.

For administration by injection (e.g., intravenous administration), the dose of mEVs (such as smEVs and/or pmEVs) can be, e.g., about 5 mg to about 900 mg total protein. The dose can be, e.g., about 20 mg to about 800 mg, about 50 mg to about 700 mg, about 75 mg to about 600 mg, about 100 mg to about 500 mg, about 250 mg to about 750 mg, or about 200 mg to about 500 mg total protein. The dose can be, e.g., about 10 mg, about 25 mg, about 50 mg, about 75 mg, about 100 mg, about 150 mg, about 200 mg, about 250 mg, about 300 mg, about 400 mg, about 500 mg, about 600 mg, or about 750 mg total protein. The dose can be, e.g., about 700 mg total protein. The dose can be, e.g., about 350 mg total protein. The dose can be, e.g., about 175 mg total protein. Total protein can be determined, e.g., by Bradford assay or BCA assay.

### Gamma-Irradiation

Powders (e.g., of mEVs (such as smEVs and/or pmEVs)) can be gamma-irradiated at 17.5 kGy radiation unit at ambient temperature.

Frozen biomasses (e.g., of mEVs (such as smEVs and/or pmEVs)) can be gamma-irradiated at 25 kGy radiation unit in the presence of dry ice.

### Additional Therapeutic Agents

In certain aspects, the methods provided herein include the administration to a subject of a pharmaceutical composition described herein either alone or in combination with an additional therapeutic agent. In some embodiments, the additional therapeutic agent is a cancer therapeutic.

In some embodiments, the pharmaceutical composition comprising mEVs (such as smEVs and/or pmEVs) from Oscillospiraceae is administered to the subject before the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, the pharmaceutical composition comprising mEVs (such as smEVs and/or pmEVs) is administered to the subject after the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the pharmaceutical composition comprising mEVs (such as smEVs and/or pmEVs) and the additional therapeutic agent are administered to the subject simultaneously or nearly simultaneously (e.g., administrations occur within an hour of each other).

In some embodiments, an antibiotic is administered to the subject before the pharmaceutical composition comprising mEVs (such as smEVs and/or pmEVs) is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before) In some embodiments, an antibiotic is administered to the subject after pharmaceutical composition comprising mEVs (such as smEVs and/or pmEVs) is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the pharmaceutical composition comprising mEVs (such as smEVs and/or pmEVs) and the antibiotic are administered to the subject simultaneously or nearly simultaneously (e.g., administrations occur within an hour of each other).

In some embodiments, the additional therapeutic agent is a cancer therapeutic. In some embodiments, the cancer therapeutic is a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxonrubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin, losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the cancer therapeutic is a cancer immunotherapy agent. Immunotherapy refers to a treatment that uses a subject's immune system to treat cancer, e.g., checkpoint inhibitors, cancer vaccines, cytokines, cell therapy, CAR-T cells, and dendritic cell therapy. Non-limiting examples of immunotherapies are checkpoint inhibitors include Nivolumab (BMS, anti-PD-1), Pembrolizumab (Merck, anti-PD-1), Ipilimumab (BMS, anti-CTLA-4), MEDI4736 (AstraZeneca, anti-PD-L1), and MPDL3280A (Roche, anti-PD-L1), Other immunotherapies may be tumor vaccines, such as Gardail, Cervarix, BCG, sipulencel-T, Gp100:209-217, AGS-003, DCVax-L, Algenpantucel-L, Tergenpantucel-L, TG4010, ProstAtak, Prostvac-V/R-TRICOM, Rindopepimul, E75 peptide acetate, IMA901, POL-103A, Belagenpumatucel-L, GSK1572932A, MDX-1279, GV 1001, and Tecemotide. The immunotherapy agent may be administered via injection (e.g., intravenously, intratumorally, subcutaneously, or into lymph nodes), but may also be administered orally, topically, or via aerosol. Immunotherapies may comprise adjuvants such as cytokines.

In some embodiments, the immunotherapy agent is an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0010718C (avelumab), AUR-012 and STI-A1010. In some embodiments, the immune checkpoint inhibitor is a CTLA-4 inhibitor. In some embodiments, the immune checkpoint inhibitor is a PD-1 inhibitor. In some embodiments, the immune checkpoint inhibitor is a PD-L1 inhibitor. In some embodiments, the immune checkpoint inhibitor is an antibody.

In some embodiments, the methods provided herein include the administration of a pharmaceutical composition described herein in combination with one or more additional therapeutic agents. In some embodiments, the methods disclosed herein include the administration of two immunotherapy agents (e.g., immune checkpoint inhibitor). For example, the methods provided herein include the administration of a pharmaceutical composition described herein in combination with a PD-1 inhibitor (such as pemrolizumab or nivolumab or pidilizumab) or a CLTA-4 inhibitor (such as ipilimumab) or a PD-L1 inhibitor.

In some embodiments, the immunotherapy agent is an antibody or antigen binding fragment thereof that, for example, binds to a cancer-associated antigen. Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML 1 fusion protein, EZH2, FGF5, FLT3-ITD, FN1, G250/MN/CAIX, GAGE-1,2,8, GAGE-3,4,5,6,7, GA S7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein. Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class 1, N-raw, NA88-A, nco-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secemin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-1b/GAGED2a. In some embodiments, the antigen is a neo-antigen.

In some embodiments, the immunotherapy agent is a cancer vaccine and/or a component of a cancer vaccine (e.g., an antigenic peptide and/or protein). The cancer vaccine can be a protein vaccine, a nucleic acid vaccine or a combination thereof. For example, in some embodiments, the cancer vaccine comprises a polypeptide comprising an epitope of a cancer-associated antigen. In some embodiments, the cancer vaccine comprises a nucleic acid (e.g., DNA or RNA, such as mRNA) that encodes an epitope of a cancer-associated antigen. Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1. Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML 1 fusion protein, EZLH2, FGF5, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1. KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, scccmin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-1b/GAGED2a. In some embodiments, the antigen is a neo-antigen. In some embodiments, the cancer vaccine is administered with an adjuvant. Examples of adjuvants include, but are not limited to, an immune modulatory protein, Adjuvant 65, α-GalCer, aluminum phosphate, aluminum hydroxide, calcium phosphate, β-Glucan Peptide, CpG ODN DNA, GPI-0100, lipid A, lipopolysaccharide, Lipovant, Montanide, N-acetyl-muramyl-L-alanyl-D-isoglutamine, Pam3CSK4, quil A, cholera toxin (CT) and heat-labile toxin from enterotoxigenic Escherichia coli (LT) including derivatives of these (CTB, mmCT, CTA1-DD, LTB, LTK63, LTR72, dmLT) and trehalose dimycolate.

In some embodiments, the immunotherapy agent is an immune modulating protein to the subject. In some embodiments, the immune modulatory protein is a cytokine or chemokine. Examples of immune modulating proteins include, but are not limited to, B lymphocyte chemoattractant (“BLC”), C-C motif chemokine 11 (“Eotaxin-1”), Easinophil chemotactic protein 2 (“Eotaxin-2”). Granulocyte colony-stimulating factor (“G-CSF”), Granulocyte macrophage colony-stimulating factor (“GM-CSF”), 1-309, Intercellular Adhesion Molecule 1 (“ICAM-1”), Interferon alpha (“IFN-alpha”), Interferon beta (“IFN-beta”) Interferon gamma (“IFN-gamma”). Interlukin-1 alpha (“IL-1 alpha”). Interlukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”). Interleukin-10 (“IL-10”). Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17A-F (“IL-17A-F”). Interleukin-18 (“IL-18”), Interleukin-21 (“IL-21”). Interleukin-22 (“IL-22”), Interleukin-23 (“IL-23”), Interleukin-33 (“IL-33”), Chemokine (C-C motif) Ligand 2 (“MCP-1”), Macrophage colony-stimulating factor (“M-CSF”), Monokine induced by gamma interferon (“MIG”). Chemokine (C-C motif) ligand 2 (“MIP-1 alpha”), Chemokine (C-C motif) ligand 4 (“MIP-1 beta”), Macrophage inflammatory protein-1-delta (“MIP-1 delta”), Platelet-derived growth factor subunit B (“PDGF-BB”), Chemokine (C-C motif) ligand 5. Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFR1”), sTNFRIIAR. Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone. Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing comples (“SCF”). Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“V EGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”). Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“1-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”). Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”). Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRG1-beta1, Beta-type platelet-derived growth factor receptor (“PDGF Rbeta”), Platelet endothelial cell adhesion molecule (“PECAM-1”). RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDARActivin A, Agouti-related protein (“AgRP”), Ribonuclease 5 (“Angiogenin”), Angiopoietin 1, Angiostatin, Catheprin S. CD40, Cryptic family protein IB (“Cripto-1”), DAN, Dickkopf-related protein 1 (“DKK-1”), E-Cadlierin, Epithehal cell adhesion molecule (“EpCAM”). Fas Ligand (FasL or CD95L), Fcg RIIB/C, Follistatin, Galectin-7, Intercellular adhesion molecule 2 (“ICAM-2”), IL-13 RI, IL-13R2, IL-17B, TL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgpl30, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”). Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”). Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-garnmalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuranoyl-L-alanine amidase (“PGRP-5”), Serpin A4, Secreted frizzled related protein 3 (“sFRP-3”), Thrombomodulin, Tolllike receptor 2 (“TLR2”), Tumor necrosis factor receptor superfamily member 10A (“TRAIL R1”), Transferrin (“TRF”), WIF-1ACE-2, Albumin. AMICA. Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL 1”), Fetuin A. Heparin-binding growth factor 1 (“aFGF”), Folate receptor alpha (“FOLR1”). Furin, GPCR-associated sorting protein 1 (“GASP-1”). GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNTI-inducible-signaling pathway protein 1 (“WISP-1”), and Receptor Activator of Nuclear Factor is B (“RANK”).

In some embodiments, the cancer therapeutic is an anti-cancer compound. Exemplary anti-cancer compounds include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzuniab (Perjeta®), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 131I-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®).

Exemplary anti-cancer compounds that modify the function of proteins that regulate gene expression and other cellular functions (e.g., HDAC inhibitors, retinoid receptor ligants) are Vorinostat (Zolinza®), Bexarotene (Targretin®) and Romidepsin (Istodax®), Alitretinoin (Panretin®), and Tretinoin (Vesanoid®).

Exemplary anti-cancer compounds that induce apoptosis (e.g., proteasome inhibitors, antifolates) are Bortezomib (Velcade®). Carfilzomib (Kyprolis™), and Pralatrexate (Folotyn®).

Exemplary anti-cancer compounds that increase anti-tumor immune response (e.g., anti CD20, anti CD52; anti-cytotoxic T-lymphocyte-associated antigen-4) are Rituximab (Rituxan®), Alemtuzumab (Campath®), Ofatumumab (Arzerra®), and Ipilimumab (Yervoy™).

Exemplary anti-cancer compounds that deliver toxic agents to cancer cells (e.g., anti-CD20-radionuclide fusions; IL-2-diphtheria toxin fusions; anti-CD30-monomethylauristatin E (MMAE)-fusions) are Tositumomab and 131I-tositumomab (Bexxar®) and Ibritumomab tiuxetan (Zevalin®), Denileukin diftitox (Ontak®), and Brentuximab vedotin (Adcetris®).

Other exemplary anti-cancer compounds are small molecule inhibitors and conjugates thereof of, e.g., Janus kinase, ALK, Bcl-2, PARP, PI3K, VEGF receptor, Braf, MEK, CDK, and HSP90.

Exemplary platinum-based anti-cancer compounds include, for example, cisplatin, carboplatin, oxaliplatin, satraplatin, picoplatin. Nedaplatin, Triplatin, and Lipoplatin. Other metal-based drugs suitable for treatment include, but are not limited to ruthenium-based compounds, ferrocene derivatives, titanium-based compounds, and gallium-based compounds.

In some embodiments, the cancer therapeutic is a radioactive moiety that comprises a radionuclide. Exemplary radionuclides include, but are not limited to Cr-51, Cs-131, Ce-134, Se-75, Ru-97, I-125, Eu-149, Os-189m, Sb-119, I-123, Ho-161, Sb-117, Ce-139, In-111, Rh-103m, Ga-67, Tl-201, Pd-103, Au-195, Hg-197, Sr-87m, Pt-191, P-33, Er-169, Ru-103, Yb-169, Au-199, Sn-121, Tm-167, Yb-175, In-113m, Sn-113, Lu-177, Rh-105, Sn-117m, Cu-67, Sc-47, Pt-195m, Ce-141, I-131, Tb-161, As-77, Pt-197, Sin-153, Gd-159, Tm-173, Pr-143, Au-198. Tm-170, Re-186, Ag-111, Pd-109, Ga-73, Dy-165, Pm-149, Sn-123, Sr-89, Ho-166, P-32, Re-188, Pr-142, Ir-194, In-114m/In-114, and Y-90.

In some embodiments, the cancer therapeutic is an antibiotic. For example, if the presence of a cancer-associated bacteria and/or a cancer-associated microbiome profile is detected according to the methods provided herein, antibiotics can be administered to eliminate the cancer-associated bacteria from the subject. “Antibiotics” broadly refers to compounds capable of inhibiting or preventing a bacterial infection. Antibiotics can be classified in a number of ways, including their use for specific infections, their mechanism of action, their bioavailability, or their spectrum of target microbe (e.g., Gram-negative vs. Gram-positive bacteria, aerobic vs. anaerobic bacteria, etc.) and these may be used to kill specific bacteria in specific areas of the host (“niches”) (Leekha, et al 2011. General Principles of Antimicrobial Therapy. Mayo Clin Proc. 86(2): 156-167). In certain embodiments, antibiotics can be used to selectively target bacteria of a specific niche. In some embodiments, antibiotics known to treat a particular infection that includes a cancer niche may be used to target cancer-associated microbes, including cancer-associated bacteria in that niche. In other embodiments, antibiotics are administered after the pharmaceutical composition comprising mEVs (such as smEVs and/or pmEVs). In some embodiments, antibiotics are administered before pharmaceutical composition comprising mEVs (such as smEVs and/or pmEVs).

In some aspects, antibiotics can be selected based on their bactericidal or bacteriostatic properties. Bactericidal antibiotics include mechanisms of action that disrupt the cell wall (e.g., β-lactams), the cell membrane (e.g., daptomycin), or bacterial DNA (e.g., fluoroquinolones). Bacteriostatic agents inhibit bacterial replication and include sulfonamides, tetracyclines, and macrolides, and act by inhibiting protein synthesis. Furthermore, while some dogs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to Amikacin, Gentamicin, Kanamycin, Neomycin, Netilmicin, Tobramycin, Paromomycin, and Spectinomycin, Aminoglycosides are effective, e.g., against Grain-negative bacteria, such as Escherichia coli, Klebsiella, Pseudomonas acruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, Geldanamycin, Herbimycin, Rifamycin, and Streptovaricin. Geldanamycin and Herbimycin are believed to inhibit or alter the function of Heat Shock Protein 90.

Carbacephems include, but are not limited to, Loracarbef. Carbacephems are believed to inhibit bacterial cell wall synthesis.

Carbapenems include, but are not limited to, Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to, Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, and Ceftobiprole. Selected Cephalosporins are effective, e.g., against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, e.g., against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin, Lincosamides are effective, e.g., against anaerobic bacteria, as well as Staphylococcus, and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, e.g., against Gram-positive bacteria Lipopeptides are believed to bind to the bacterial membrane and cause rapid depolarization.

Macrolides include, but are not limited to, Azithromycin, Clarithromycin, Dirithromycin, Erythromycin, Rorithromycin, Troleandomycin, Telithromycin. and Spiramycin. Macrolides are effective, e.g., against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective, e.g., against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to, Linezolid, Posizolid, Radezolid, and Torezolid. Oxazolidonones are believed to be protein synthesis inhibitors.

Penicillins include, but are not limited to. Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin and Ticarcillin. Penicillins are effective. e.g., against Gram-positive bacteria, facultative anaerobes, e.g., Streptococcus, Borrelia, and Treponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to. Bacitracin, Colistin, and Polymyxin B and E. Polypeptide Antibiotics are effective, e.g., against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, e.g., against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, e.g., against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide, Isoniazid, Pyrazinamide, Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillin/clavulanate, ampicillin/sulbactam, amphomycin ristocetin, azithromycin, bacitracin, buforin II, carbomycin, cecropin PI, clarithromycin, erythromycins, furazolidone, fusidic acid. Nafusidate, gramicidin, imipenem, indolicidin, josamycin, magainan II, metronidazole, nitroimidazoles, mikamycin, mutacin B-Ny266, mutacin B-JHI 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

### Administration

In certain aspects, provided herein is a method of delivering a pharmaceutical composition described herein (e.g., a pharmaceutical composition comprising mEVs (such as smEVs and/or pmEVs from Oscillospiraceae) to a subject. In some embodiments of the methods provided herein, the pharmaceutical composition is administered in conjunction with the administration of an additional therapeutic agent. In some embodiments, the pharmaceutical composition comprises mEVs (such as smEVs and/or pmEVs) co-formulated with the additional therapeutic agent. In some embodiments, the pharmaceutical composition comprising mEVs (such as smEVs and/or pmEVs) is co-administered with the additional therapeutic agent. In some embodiments, the additional therapeutic agent is administered to the subject before administration of the pharmaceutical composition that comprises mEVs (such as smEVs and/or pmEVs) (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic agent is administered to the subject after administration of the pharmaceutical composition that comprises mEVs (such as smEVs and/or pmEVs) (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments, the same mode of delivery is used to deliver both the pharmaceutical composition that comprises mEVs (such as smEVs and/or pmEVs) and the additional therapeutic agent. In some embodiments, different modes of delivery are used to administer the pharmaceutical composition that comprises mEVs (such as smEVs and/or pmEVs) and the additional therapeutic agent. For example, in some embodiments the pharmaceutical composition that comprises mEVs (such as smEVs and/or pmEVs) is administered orally while the additional therapeutic agent is administered via injection (e.g., an intravenous, intramuscular and/or intratumoral injection). In some embodiments, the pharmaceutical composition described herein is administered once a day. In some embodiments, the pharmaceutical composition described herein is administered twice a day. In some embodiments, the pharmaceutical composition described herein is formulated for a daily dose. In some embodiments, the pharmaceutical composition described herein is formulated for twice a day dose, wherein each dose is half of the daily dose.

In certain embodiments, the pharmaceutical compositions and dosage forms described herein can be administered in conjunction with any other conventional anti-cancer treatment, such as, for example, radiation therapy and surgical resection of the tumor. These treatments may be applied as necessary and/or as indicated and may occur before, concurrent with or after administration of the pharmaceutical composition that comprises mEVs (such as smEVs and/or pmEVs) or dosage forms described herein.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, for example, tumor size, and other compounds such as drugs being administered concurrently or near-concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of a pharmaceutical composition that comprises mEVs (such as smEVs and/or pmEVs) described herein may be appropriately set or adjusted in accordance with the dosage form, the route of administration, the degree or stage of a target disease, and the like. For example, the general effective dose of the agents may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to prevent disease (e.g., a cancer, an autoimmune disease, an inflammatory disease, a dysbiosis, or a metabolic disease), delay its onset, or slow or stop its progression, or relieve one or more symptoms of the disease. One skilled in the art will recognize that dosage will depend upon a variety of factors including the strength of the particular agent (e.g., therapeutic agent) employed, as well as the age, species, condition, and body weight of the subject. The size of the dose will also be determined by the route, timing, and frequency of administration as well as the existence, nature, and extent of any adverse side-effects that might accompany the administration of a particular therapeutic agent and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the therapeutic agents used in accordance with the invention vary depending on the active agent, the age, weight, and clinical condition of the recipient patient, and the experience and judgment of the clinician or practitioner administering the therapy, among other factors affecting the selected dosage. For example, for cancer treatment, the dose should be sufficient to result in slowing, and preferably regressing, the growth of a tumor and most preferably causing complete regression of the cancer, or reduction in the size or number of metastases. As another example, the dose should be sufficient to result in slowing of progression of the disease for which the subject is being treated, and preferably amelioration of one or more symptoms of the disease for which the subject is being treated.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a pharmaceutical composition, where the number of administrations can be determined by monitoring the subject, and, based on the results of the monitoring, determining whether or not to provide one or more additional administrations Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response. In one example, the time period can be a function of the time period for a subject to mount an immune response: for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month.

In some embodiments, the delivery of an additional therapeutic agent in combination with the pharmaceutical composition described herein reduces the adverse effects and/or improves the efficacy of the additional therapeutic agent.

The effective dose of an additional therapeutic agent described herein is the amount of the additional therapeutic agent that is effective to achieve the desired therapeutic response for a particular subject, composition, and mode of administration, with the least toxicity to the subject. The effective dosage level can be identified using the methods described herein and will depend upon a variety of pharmacokinetic factors including the activity of the particular compositions or agents administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts. In general, an effective dose of an additional therapeutic agent will be the amount of the additional therapeutic agent which is the lowest dose effective to produce a therapeutic effect. Such an effective dose will generally depend upon the factors described above.

The toxicity of an additional therapeutic agent is the level of adverse effects experienced by the subject during and following treatment. Adverse events associated with additional therapy toxicity can include, but are not limited to, abdominal pain, acid indigestion, acid reflux, allergic reactions, alopecia, anaphylasix, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypematremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myclosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heart beat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

### Metabolic Disorders

In some embodiments, the methods and pharmaceutical compositions described herein relate to the treatment or prevention of a metabolic disease or disorder a, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), Nonalcoholic Steatohepatitis (NASH) or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema. In some embodiments, the methods and pharmaceutical compositions described herein relate to the treatment of Nonalcoholic Fatty Liver Disease (NAFLD) and Nonalcoholic Steatohepatitis (NASH).

The methods described herein can be used to treat any subject in need thereof. As used herein, a “subject in need thereof” includes any subject that has a metabolic disease or disorder, as well as any subject with an increased likelihood of acquiring a such a disease or disorder.

The pharmaceutical compositions described herein can be used, for example, for preventing or treating (reducing, partially or completely, the adverse effects of) a metabolic disease, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), Nonalcoholic Steatohepatitis (NASH), or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema.

### Immune Disorders

In some embodiments, the methods and pharmaceutical compositions described herein relate to the treatment or prevention of a disease or disorder associated a pathological immune response, such as an autoimmune disease, an allergic reaction and/or an inflammatory disease. In some embodiments, the disease or disorder is an inflammatory bowel disease (e.g., Crohn's disease or ulcerative colitis). In some embodiments, the disease or disorder is psoriasis. In some embodiments, the disease or disorder is atopic dermatitis.

The methods described herein can be used to treat any subject in need thereof. As used herein, a “subject in need thereof” includes any subject that has a disease or disorder associated with a pathological immune response (e.g., an inflammatory bowel disease), as well as any subject with an increased likelihood of acquiring a such a disease or disorder.

The pharmaceutical compositions described herein can be used, for example, as a pharmaceutical composition for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto's disease; an allergic disease, such as a food allergy, pollenosis, or asthma; an infectious disease, such as an infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur; a supplement, food, or beverage for improving immune functions; or a reagent for suppressing the proliferation or function of immune cells.

In some embodiments, the methods provided herein are useful for the treatment of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular immune disorders refers to a immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis.

Examples of nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and pharmaceutical compositions described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the methods and pharmaceutical compositions described herein include, but are not limited to, cervicitis, chorioamnionitis, endometritis, epididymitis, omphalitis, oophoritis, orchitis, salpingitis, tubo-ovarian abscess, urcthritis, vaginitis, vulvitis, and vulvodynia.

The methods and pharmaceutical compositions described herein may be used to treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, goodpasture's syndrome. Grave's disease. Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and pharmaceutical compositions described heroin may be used to treat T-cell mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dustmite allergy) and gluten-sensitive enteropathy (Celiac disease).

Other immune disorders which may be treated with the methods and pharmaceutical compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### Cancer

In some embodiments, the methods and pharmaceutical compositions described herein relate to the treatment of cancer. In some embodiments, any cancer can be treated using the methods described herein. Examples of cancers that may treated by methods and pharmaceutical compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma, papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp, adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma, oligodendroblastoma; primitive neuroectodermal, cerebellar sarcoma, ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma, mast cell sarcoma, immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the methods and pharmaceutical compositions provided herein relate to the treatment of a leukemia. The term “leukemia” includes broadly progressive, malignant diseases of the hematopoietic organs/systems and is generally characterized by a distorted proliferation and development of leukocytes and their precursors in the blood and bone marrow. Non-limiting examples of leukemia diseases include, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

In some embodiments, the methods and pharmaceutical compositions provided herein relate to the treatment of a carcinoma. The term “carcinoma” refers to a malignant growth made up of epithelial cells tending to infiltrate the surrounding tissues, and/or resist physiological and non-physiological cell death signals and gives rise to metastases. Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

In some embodiments, the methods and pharmaceutical compositions provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the methods and pharmaceutical compositions described herein include Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, plasmacytoma, colorectal cancer, rectal cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

In some embodiments, the cancer comprises breast cancer (e.g., triple negative breast cancer).

In some embodiments, the cancer comprises colorectal cancer (e.g., microsatellite stable (MSS) colorectal cancer).

In some embodiments, the cancer comprises renal cell carcinoma.

In some embodiments, the cancer comprises lung cancer (e.g., non small cell lung cancer).

In some embodiments, the cancer comprises bladder cancer.

In some embodiments, the cancer comprises gastroesophageal cancer.

Particular categories of tumors that can be treated using methods and pharmaceutical compositions described herein include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, plasmacytoma, colorectal cancer, and rectal cancer.

Cancers treated in certain embodiments also include precancerous lesions, e.g., actinic keratosis (solar keratosis), moles (dysplastic nevi), acitinic chelitis (farmer's lip), cutaneous horns. Barrett's esophagus, atrophic gastritis, dyskeratosis congenita, sideropenic dysphagia, lichen planus, oral submucous fibrosis, actinic (solar) elastosis and cervical dysplasia.

Cancers treated in some embodiments include non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

In some embodiments, the cancer comprises a solid tumor.

### Dysbiosis

In recent years, it has become increasingly clear that the gut microbiome (also called the “gut microbiota”) can have a significant impact on an individual's health through microbial activity and influence (local and/or distal) on immune and other cells of the host (Walker, W. A., Dysbiosis. The Microbiota in Gastrointestinal Pathophysiology. Chapter 25, 2017; Weiss and Thierry, Mechanisms and consequences of intestinal dysbiosis. Cellular and Molecular Life Sciences. (2017) 74(16):2959-2977. Zurich Open Repository and Archive).

A healthy host-gut microbiome homeostasis is sometimes referred to as a “cubiosis” or “normobiosis,” whereas a detrimental change in the host microbiome composition and/or its diversity can lead to an unhealthy imbalance in the microbiome, or a “dysbiosis” (Hooks and O'Malley. Dysbiosis and its discontents. American Society for Microbiology. October 2017. Vol. 8. Issue 5, mBio 8:e01492-17). Dysbiosis, and associated local or distal host inflammatory or immune effects, may occur where microbiome homeostasis is lost or diminished, resulting in: increased susceptibility to pathogens; altered host bacterial metabolic activity; induction of host proinflammatory activity and/or reduction of host anti-inflammatory activity. Such effects are mediated in part by interactions between host immune cells (e.g., T cells, dendritic cells, mast cells, NK cells, intestinal epithelial lymphocytes (IEC), macrophages and phagocytes) and cytokines, and other substances released by such cells and other host cells.

A dysbiosis may occur within the gastrointestinal tract (a “gastrointestinal dysbiosis” or “gut dysbiosis”) or may occur outside the lumen of the gastrointestinal tract (a “distal dysbiosis”). Gastrointestinal dysbiosis is often associated with a reduction in integrity of the intestinal epithelial barrier, reduced tight junction integrity and increased intestinal permeability. Citi, S. Intestinal Barriers protect against disease, Science 359:1098-99 (2018); Srinivasan et al., TEER measurement techniques for in vitro barrier model systems. J. Lab. Autom. 20:107-126 (2015). A gastrointestinal dysbiosis can have physiological and immune effects within and outside the gastrointestinal tract.

The presence of a dysbiosis has been associated with a wide variety of diseases and conditions including: infection, cancer, autoimmune disorders (e.g., systemic lupus erythematosus (SLE)) or inflammatory disorders (e.g., functional gastrointestinal disorders such as inflammatory bowel disease (IBD), ulcerative colitis, and Crohn's disease), neuroinflammatory diseases (e.g., multiple sclerosis), transplant disorders (e.g., graft-versus-host disease), fatty liver disease, type I diabetes, rheumatoid arthritis, Sjögren's syndrome, celiac disease, cystic fibrosis, chronic obstructive pulmonary disorder (COPD), and other diseases and conditions associated with immune dysfunction. Lynch et al., The Human Microbiome in Health and Disease, N. Engl. J. Med 375:2369-79 (2016), Carding et al., Dysbiosis of the gut microbiota in disease. Microb. Ecol. Health Dis. (2015): 26: 10: 3402/mehd.v26.2619; Levy et al, Dysbiosis and the Immune System, Nature Reviews Immunology 17:219 (April 2017).

Exemplary pharmaceutical compositions disclosed herein can treat a dysbiosis and its effects by modifying the immune activity present at the site of dysbiosis. As described herein, such compositions can modify a dysbiosis via effects on host immune cells, resulting in. e.g., an increase in secretion of anti-inflammatory cytokines and/or a decrease in secretion of pro-inflammatory cytokines, reducing inflammation in the subject recipient or via changes in metabolite production.

Exemplary pharmaceutical compositions disclosed herein that are useful for treatment of disorders associated with a dysbiosis contain one or more types of immunomodulatory bacteria (e.g., anti-inflammatory bacteria) and/or mEVs (microbial extracellular vesicles) derived from such bacteria. Such compositions are capable of affecting the recipient host's immune function, in the gastrointestinal tract, and/or a systemic effect at distal sites outside the subject's gastrointestinal tract.

Exemplary pharmaceutical compositions disclosed herein that are useful for treatment of disorders associated with a dysbiosis contain a population of Oscillospiraceae bacteria of a single bacterial species (e.g., a single strain) (e.g., anti-inflammatory bacteria) and/or mEVs derived from such bacteria. Such compositions are capable of affecting the recipient host's immune function, in the gastrointestinal tract, and/or a systemic effect at distal sites outside the subject's gastrointestinal tract.

In some embodiments, pharmaceutical compositions containing an isolated population of Oscillospiraceae bacteria (e.g., anti-inflammatory bacterial cells) or mEVs derived from such bacteria are administered (e.g., orally) to a mammalian recipient in an amount effective to treat a dysbiosis and one or more of its effects in the recipient. The dysbiosis may be a gastrointestinal tract dysbiosis or a distal dysbiosis.

In another embodiment, pharmaceutical compositions of the instant invention can treat a gastrointestinal dysbiosis and one or more of its effects on host immune cells, resulting in an increase in secretion of anti-inflammatory cytokines and/or a decrease in secretion of pro-inflammatory cytokines, reducing inflammation in the subject recipient.

In another embodiment, the pharmaceutical compositions can treat a gastrointestinal dysbiosis and one or more of its effects by modulating the recipient immune response via cellular and cytokine modulation to reduce gut permeability by increasing the integrity of the intestinal epithelial barrier.

In another embodiment, the pharmaceutical compositions can treat a distal dysbiosis and one or more of its effects by modulating the recipient immune response at the site of dysbiosis via modulation of host immune cells.

Other exemplary pharmaceutical compositions are useful for treatment of disorders associated with a dysbiosis, which compositions contain one or more types of Oscillospiraceae bacteria or mEVs capable of altering the relative proportions of host immune cell subpopulations, e.g., subpopulations of T cells, immune lymphoid cells, dendritic cells, NK cells and other immune cells, or the function thereof, in the recipient.

Other exemplary pharmaceutical compositions are useful for treatment of disorders associated with a dysbiosis, which compositions contain a population of Oscillospiraceae bacteria or mEVs of a single bacterial species e.g., a single strain) capable of altering the relative proportions of immune cell subpopulations, e.g., T cell subpopulations, immune lymphoid cells, NK cells and other immune cells, or the function thereof, in the recipient subject. In some embodiments, Oscillospiraceae strain is Faecalibacterium prausnitzii (e.g., Faecalibacterium prausnitzii strain A), Fournierella massiliensis (e.g., Fournierella massiliensis strain A), Harryflintia acetispora (e.g., Harryflintia acetispora strain A), Agathobaculum sp. (e.g., Agathobaculum sp. strain A), Acutalibacter sp. (e.g., Acutalibacter sp. strain A), Anaerotruncus colihominis (Anaerotruncus colihominis strain A), or Subdoligranulum variabile (e.g., Subdoligranulum variabile strain A).

In some embodiments, the invention provides methods of treating a gastrointestinal dysbiosis and one or more of its effects by orally administering to a subject in need thereof a pharmaceutical composition described herein which alters the microbiome population existing at the site of the dysbiosis. The pharmaceutical composition can contain one or more types of Oscillospiraceae bacteria or mEVs or a population of Oscillospiraceae bacteria or mEVs of a single bacterial species (e.g., a single strain). In some embodiments. Oscillospiraceae strain is Faecalibacterium prausnitzii (e.g., Faecalibacterium prausnitzii strain A), Fournierella massiliensis (e.g., Fournierella massiliensis strain A). Harryflintia acetispora (e.g., Harryflintia acetispora strain A), Agathobaculum sp. (e.g., Agathobaculum sp. strain A), Acutalibacter sp. (e.g., Acutalibacter sp. strain A), Anaerotruncus colihominis (Anaerotruncus colihominis strain A), or Subdoligranulum variabile (e.g., Subdoligranulum variabile strain A).

In some embodiments, the invention provides methods of treating a distal dysbiosis and one or more of its effects by orally administering to a subject in need thereof a pharmaceutical composition described herein which alters the subject's immune response outside the gastrointestinal tract. The pharmaceutical composition can contain one or more types of Oscillospiraceae bacteria or mEVs or a population of Oscillospiraceae bacteria or mEVs of a single bacterial species (e.g., a single strain). In some embodiments, Oscillospiraceae strain is Faecalibacterium prausnitzii (e.g., Faecalibacterium prausnitzii strain A), Fournierella massiliensis (e.g., Fournierella massiliensis strain A), Harryflintia acetispora (e.g., Harryflintia acetispora strain A), Agathobaculum sp. (e.g., Agathobaculum sp. strain A), Acutalibacter sp. (e.g., Acutalibacter sp. strain A), Anaerotruncus colihominis (Anaerotruncus colihominis strain A), or Subdoligranulum variabile (e.g., Subdoligranulum variabile strain A).

In exemplary embodiments, pharmaceutical compositions useful for treatment of disorders associated with a dysbiosis stimulate secretion of one or more anti-inflammatory cytokines by host immune cells. Anti-inflammatory cytokines include, but are not limited to, IL-10, IL-13, IL-9, IL-4, IL-5, TGFβ, and combinations thereof. In other exemplary embodiments, pharmaceutical compositions useful for treatment of disorders associated with a dysbiosis that decrease (e.g., inhibit) secretion of one or more pro-inflammatory cytokines by host immune cells. Pro-inflammatory cytokines include, but are not limited to: IFNγ, IL-12p70, IL-1α, IL-6, IL-8, MCP1, MIP1α, MIP1β, TNFα, and combinations thereof. Other exemplary cytokines are known in the art and are described herein.

In another aspect, the invention provides a method of treating or preventing a disorder associated with a dysbiosis in a subject in need thereof, comprising administering (e.g., orally administering) to the subject a therapeutic composition in the form of a probiotic or medical food comprising bacteria or mEVs in an amount sufficient to alter the microbiome at a site of the dysbiosis, such that the disorder associated with the dysbiosis is treated.

In another embodiment, a therapeutic composition of the instant invention in the form of a probiotic or medical food may be used to prevent or delay the onset of a dysbiosis in a subject at risk for developing a dysbiosis.

### Other Diseases and Disorders

In some embodiments, the methods and pharmaceutical compositions described herein relate to the treatment of liver diseases. Such diseases include, but are not limited to, Alagille Syndrome, Alcohol-Related Liver Disease, Alpha-1 Antitrypsin Deficiency, Autoimmune Hepatitis, Benign Liver Tumors, Biliary Atresia, Cirrhosis, Galactosemia, Gilbert Syndrome, Hemochromatosis, Hepatitis A, Hepatitis B, Hepatitis C, Hepatic Encephalopathy, Intrahepatic Cholestasis of Pregnancy (ICP), Lysosomal Acid Lipase Deficiency (LAL-D), Liver Cysts. Liver Cancer, Newborn Jaundice, Primary Biliary Cholangitis (PBC), Primary Sclerosing Cholangitis (PSC), Rcyc Syndrome, Type I Glycogen Storage Disease, and Wilson Disease.

The methods and pharmaceutical compositions described herein may be used to treat neurodegenerative and neurological diseases. In certain embodiments, the neurodegenerative and/or neurological disease is Parkinson's disease, Alzheimer's disease, prion disease, Huntington's disease, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy or post-operative cognitive dysfunction.

### Methods of Making Enhanced Bacteria

In certain aspects, provided herein are methods of making engineered bacteria for the production of the mEVs (such as smEVs and/or pmEVs) described herein. In some embodiments, the engineered bacteria are modified to enhance certain desirable properties. For example, in some embodiments, the engineered bacteria are modified to enhance the immunomodulatory and/or therapeutic effect of the mEVs (such as smEVs and/or pmEVs) (e.g., either alone or in combination with another therapeutic agent), to reduce toxicity and/or to improve bacterial and/or mEV (such as smEV and/or pmEV) manufacturing (e.g., higher oxygen tolerance, improved freeze-thaw tolerance, shorter generation times). The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, CRISPR/Cas9, or any combination thereof.

In some embodiments of the methods provided herein, the bacterium is modified by directed evolution. In some embodiments, the directed evolution comprises exposure of the bacterium to an environmental condition and selection of bacterium with improved survival and/or growth under the environmental condition. In some embodiments, the method comprises a screen of mutagenized bacteria using an assay that identifies enhanced bacterium. In some embodiments, the method further comprises mutagenizing the bacteria (e.g., by exposure to chemical mutagens and/or UV radiation) or exposing them to a therapeutic agent (e.g., antibiotic) followed by an assay to detect bacteria having the desired phenotype (e.g., an in vivo assay, an ex vivo assay, or an in vitro assay).

### EXAMPLES

### Example 1: Cultivation and Storage Conditions for Oscillospiraceae Strains

Anaerobic Tryptic Soy Broth (TSB) medium supplemented with Hemoglobin.

Composition (g/L):

| | | | |
|---|---|---|---|
| 1. | Pancreatic Digest of Casein | 17.0 | g |
| 2. | Peptic Digest of Soybean Meal | 3.0 | g |
| 3. | Dipotassium Phosphate K2HPO4 | 2.5 | g |
| 4. | Yeast Extract | 5.0 | g |
| 5. | Dextrose | 2.5 | g |
| 6. | Sodium Chloride | 5.0 | g |
| 7. | L-Cysteine-HCL | 0.5 | g |
| 8. | FeCl₂ | 0.05 | g |
| 9. | Hemoglobin | 0.02 | g |

Final pH 6.8+/−0.3 at 25 degrees C.

Reducing agent 100× preparation:
a. Dissolve 5 g of L-Cysteine-HCl in 100 ml of H20
b. Add 0.5 g of FcCl2
c. Store solution under anaerobic conditions

Hemoglobin solution 100× preparation:
a. Dissolve 2 g of Hemoglobin in 100 ml of 0.01N NaOH
b. Autoclave the solution
c. Store the solution protected from direct light at 4 C
d. Add the solution to sterilized medium
   Oscillospiraceae strains (e.g., Fournierella massiliensis strain A) is able to grow without hemoglobin in liquid TSB medium if single colony is transferred from Brucella blood plate.
   Anaerobic Brucella Blood Alar Plates. Anaerobe Systems #AS-141.

Anaerobic glycerol (GSS) as cryoprotectant, Anaerobe Systems #AS-9045:

Composition (g/L):
1. Glycerol 400 g
2. Magnesium Sulfate. Heptahydrate 0.1 g
3. Potassium Phosphate, Monobasic 0.2 g
4. Potassium Chloride 0.2 g
5. Sodium Phosphate, Dibasic 1.15 g
6. Sodium Chloride 3.0 g
7. Sodium Thioglycollate 1.0 g
8. L-Cysteine (25.0% solution) 2.0 mL
Mix anaerobic glycerol with bacterial suspension 1:1 by volume, snap-freeze and store at −80° C.; Incubation at 37° C. for 1-3 days; Anaerobic conditions: a) for agar plates in Mitsubishi anaerobic jars with anaerobic gas pack; b) for liquid culture in closed anaerobic Hungate or Balch test tubes.

### Example 2: Oral Delivery of F. massiliensis smEVs Strain A in CT26 Tumor Studies

### First Representative Oncology Study

Female 8 week old BALB/c mice were acquired from Taconic Biosciences and allowed to acclimate at a vivarium for 3 weeks. On Day 0, mice were anesthetized with isoflurane, and inoculated subcutaneously on the left flank with 1.0e5 CT-26 cells (0.1 mL) prepared in PBS and Corning (GFR) Phenol Red-Free Matrigel (1:1). Mice were allowed to rest for 9 days post CT-26 inoculation to allow formation of palpable tumors. On Day 9, tumors were measured using a sliding digital caliper to collect length and width in measurements (in millimeters) to calculate estimated tumor volume ((L×W×W)/2)=TVmm3)). Mice were randomized into different treatment groups with a total of 9 or 10 mice per group. Randomization was done to balance all treatment groups, allowing each group to begin treatment with a similar average tumor volume and standard deviation. Dosing began on Day 10, and ended on Day 22 for 13 consecutive days of dosing. Mice were orally dosed BID with F. massiliensis strain A smEVs, or Q4D intraperitoneally with 200 ug anti-mouse PD-1 antibody. Body weight and tumor measurements were collected on a MWF (Monday-Wednesday-Friday) schedule. Dose of smEVs was determined by particle count by NTA.

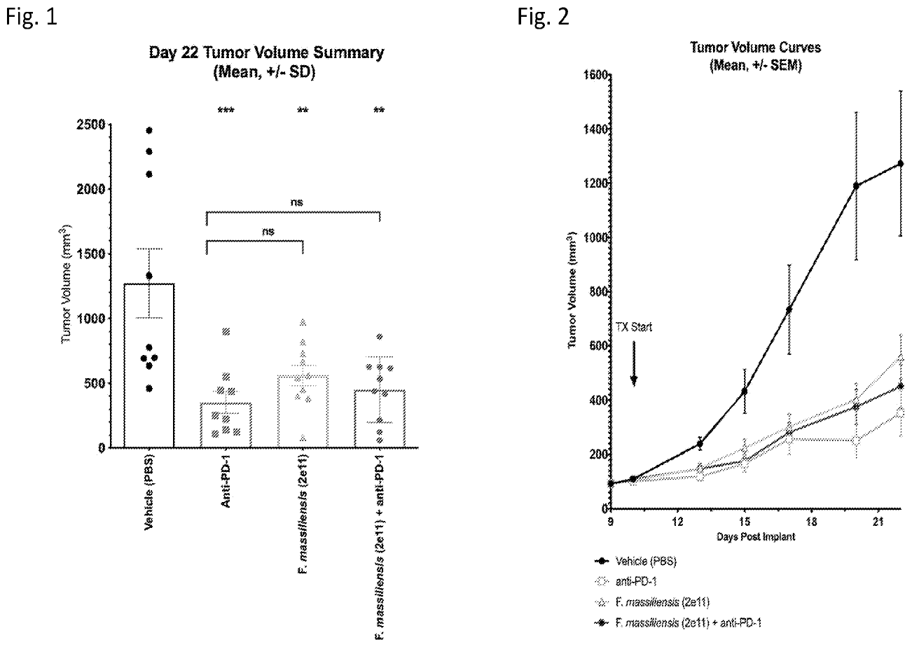

The Day 22 Tumor Volume Summary in FIG. 1 compares F. massiliensis smEV (2e11) and F. massiliensis smEV (2e11)+anti-PD-1 against a negative control (Vehicle PBS), and positive control (anti-PD-1). Both F. massiliensis smEV treatment groups compared to Vehicle PBS showed statistically significant efficacy and are not significantly different than anti-PD-1. The Tumor Volume Curves in FIG. 2 show similar growth trends for both F. massiliensis smEV groups and anti-PD-1, along with sustained efficacy after 13 days of treatment.

### Second Representative Oncology Study

Female 8 week old BALB/c mice were acquired from Taconic Biosciences and allowed to acclimate at a vivarium for 1 week. On Day 0, mice were anesthetized with isoflurane, and inoculated subcutaneously on the left flank with 1.0e CT-26 cells (0.1 mL) prepared in PBS and Corning (GFR) Phenol Red-Free Matrigel (1:1). Mice were allowed to rest for 9 days post CT-26 inoculation to allow formation of palpable tumors. On Day 9, tumors were measured using a sliding digital caliper to collect length and width in measurements (in millimeters) to calculate estimated tumor volume ((L×W×W)/2)=TVmm3)). Mice were randomized into different treatment groups with a total of 9 mice per group. Randomization was done to balance all treatment groups, allowing each group to begin treatment with a similar average tumor volume and standard deviation. Dosing began on Day 10, and ended on Day 23 for 14 consecutive days of dosing. Mice were orally dosed BID with F. massiliensis strain A smEVs, or Q4D intraperitoneally with 200 ug anti-mouse PD-1 antibody. Body weight and tumor measurements were collected on a MWF schedule. Dose of smEVs was determined by particle count by NTA.

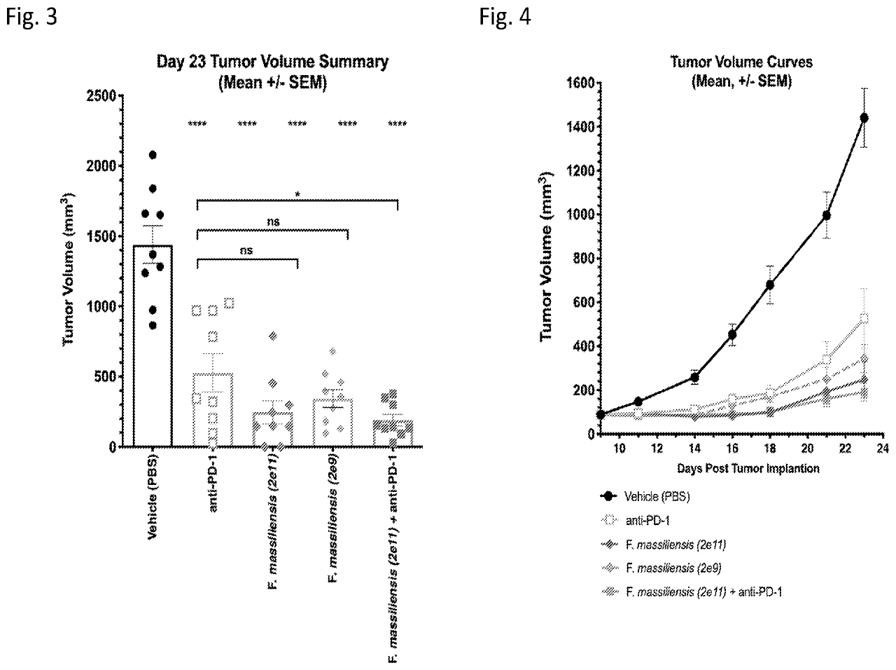

The Day 23 Tumor Volume Summary in FIG. 3 compares F. massiliensis smEV (2e11), F. massiliensis smEV (2e9), and F. massiliensis smEV (2e11)+anti-PD-1 against a negative control (Vehicle PBS), and positive control (anti-PD-1). All F. massiliensis smEV treatment groups compared to Vehicle PBS show statistically significant efficacy compared to Vehicle (PBS) Both doses of F. massiliensis smEV (2e9 & 2e11) are not significantly different than anti-PD-1. F. massiliensis smEV (2e11)+anti-PD-1 shows statistically improved efficacy over anti-PD-1 treatment alone. The Tumor Growth Curve in FIG. 4 shows sustained efficacy of F. massiliensis smEV treatment groups over 14 days of treatment similar to anti-PD-1.

### Third Representative Oncology Study

Female 8 week old BALB/c mice were acquired from Taconic Biosciences and allowed to acclimate at a vivarium for 1 week. On Day 0, mice were anesthetized with isoflurane, and inoculated subcutaneously on the left flank with 1.0e5 CT-26 cells (0.1 mL) prepared in PBS and Corning (GFR) Phenol Red-Free Matrigel (1:1). Mice were allowed to rest for 9 days post CT-26 inoculation to allow formation of palpable tumors. On Day 9, tumors were measured using a sliding digital caliper to collect length and width in measurements (in millimeters) to calculate estimated tumor volume ((L×W×W)/2)=TVmm3)). Mice were randomized into different treatment groups with a total of 9 mice per group. Randomization was done to balance all treatment groups, allowing each group to begin treatment with a similar average tumor volume and standard deviation. Dosing began on Day 10, and ended on Day 21 for 12 consecutive days of dosing. Mice were orally dosed QD and BID respectively with F. massiliensis strain A smEVs, or Q4D intraperitoneally with 200 ug anti-mouse PD-1 antibody. Body weight and tumor measurements were collected on a MWF schedule. Dose of smEVs was determined by particle count by NTA.

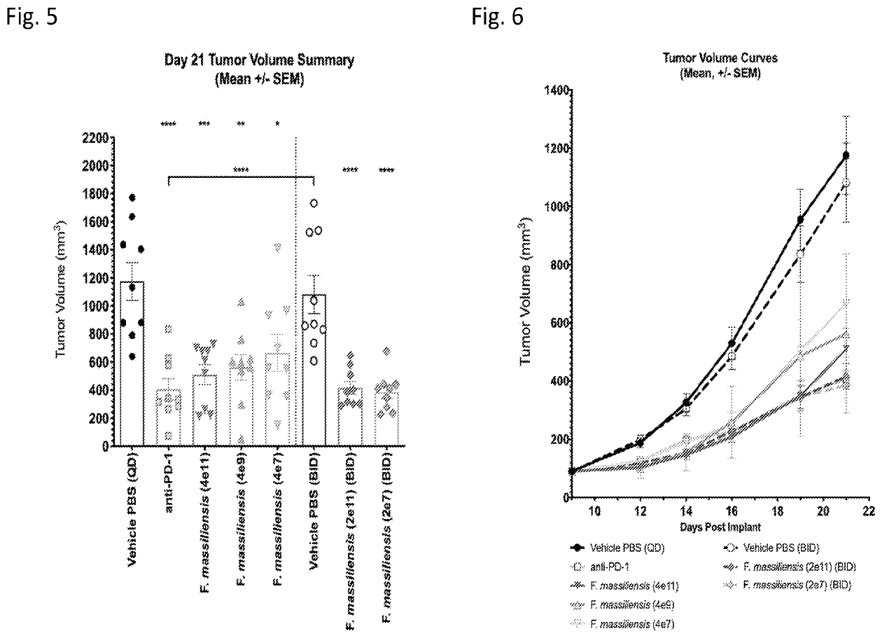

The Day 21 Tumor Volume Summary in FIG. 5 on the left, compares F. massiliensis smEVs at 3 doses (4e11, 4e9, & 4e7), administered QD for 12 days against respective negative control (Vehicle PBS) and positive control (anti-PD-1). The Tumor Volume Summary in FIG. 5 also compares F. massiliensis smEVs at 2 doses (2e11 & 2e7) administered BID for a total daily dose of 4e11 & 4e7 respectively against their corresponding negative control (Vehicle PBS BID) and positive control (anti-PD-1).

The QD F. massiliensis smEV treatment arms compared to Vehicle PBS show an evident dose effect with increased significance and efficacy trending towards the high dose of F. massiliensis smEV at (4e11). The BID F. massiliensis smEV treatment arms compared to Vehicle PBS BID show significant efficacy with no evidence of a dose effect. This result indicates both doses BID 2e11 & 2e7 are comparable and show an efficacious advantage dosing BID versus QD. The Tumor Growth Curve in FIG. 6 shows a sustained dose effect for the QD arm after 12 days of dosing, as well as comparable growth trends for the BID treatment arms compared to F. massiliensis smEV (4e11) QD.

### Example 3: Oral Delivery of H. acetispora smEVs Strain A in CT26 Tumor Studies

### First Representative Oncology Study

Female 8 week old BALB/c mice were acquired from Taconic Biosciences and allowed to acclimate at a vivarium for 3 weeks. On Day 0, mice were anesthetized with isoflurane, and inoculated subcutaneously on the left flank with 1.0e5 CT-26 cells (0.1 mL) prepared in PBS and Corning (GFR) Phenol Red-Free Matrigel (1.1). Mice were allowed to rest for 9 days post CT-26 inoculation to allow formation of palpable tumors. On Day 9, tumors were measured using a sliding digital caliper to collect length and width in measurements (in millimeters) to calculate estimated tumor volume ((L×W×W)/2)=TVmm3)). Mice were randomized into different treatment groups with a total of 9 or 10 mice per group. Randomization was done to balance all treatment groups, allowing each group to begin treatment with a similar average tumor volume and standard deviation. Dosing began on Day 10, and ended on Day 22 for 13 consecutive days of dosing. Mice were orally dosed BID with H. acetispora strain A smEVs, or Q4D intraperitoneally with 200 ug anti-mouse PD-1 antibody. Body weight and tumor measurements were collected on a MWF (Monday-Wednesday-Friday) schedule. Dose of smEVs was determined by particle count by NTA.

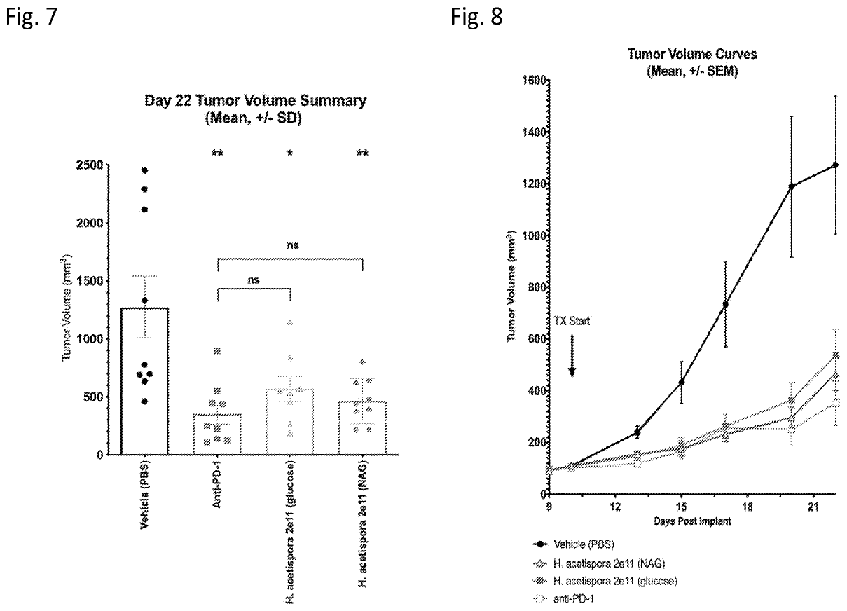

The Day 22 Tumor Volume Summary in FIG. 7 compares H. acetispora (2e11) smEV (growth media containing glucose) and H. acetispora (2e11) smEV (growth media 2 containing NAG) against a negative control (Vehicle PBS), and positive control (anti-PD-1). Both H. acetispora treatment groups compared to Vehicle PBS showed statistically significant efficacy and are not significantly different than anti-PD-1. The Tumor Volume Curves in FIG. 8 show similar growth trends for both H. acetispora groups and anti-PD-1, along with sustained efficacy after 13 days of treatment.

### Second Representative Oncology Study

Female 8 week old BALB/c mice were acquired from Taconic Biosciences and allowed to acclimate at a vivarium for 1 week. On Day 0, mice were anesthetized with isoflurane, and inoculated subcutaneously on the left flank with 1.0e5 CT-26 cells (0.1 mL) prepared in PBS and Corning (GFR) Phenol Red-Free Matrigel (1:1). Mice were allowed to rest for 9 days post CT-26 inoculation to allow formation of palpable tumors. On Day 9, tumors were measured using a sliding digital caliper to collect length and width in measurements (in millimeters) to calculate estimated tumor volume ((L×W×W)/2)=TVmm3)). Mice were randomized into different treatment groups with a total of 9 mice per group. Randomization was done to balance all treatment groups, allowing each group to begin treatment with a similar average tumor volume and standard deviation. Dosing began on Day 10, and ended on Day 23 for 14 consecutive days of dosing. Mice were orally dosed BID with H. acetispora Strain A smEVs, or Q4D intraperitoneally with 200 ug anti-mouse PD-1 antibody. Body weight and tumor measurements were collected on a MWF schedule. Dose of smEVs was determined by particle count by NTA.

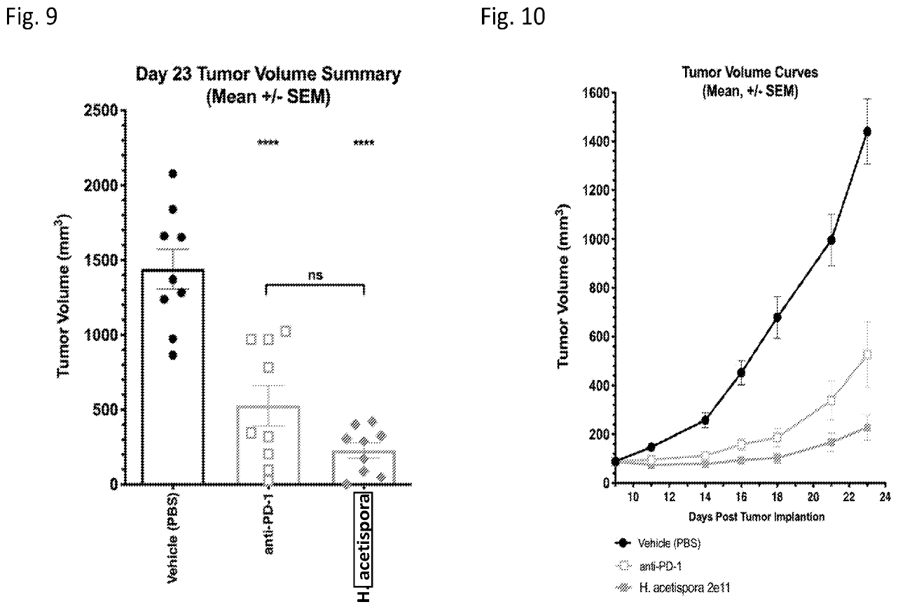

The Day 23 Tumor Volume Summary in FIG. 9 compares H. acetispora smEV (2e11) against a negative control (Vehicle PBS), and positive control (anti-PD-1). The H. acetispora smEV (2e11) treatment groups compared to Vehicle PBS shows statistically significant efficacy compared to Vehicle (PBS), and is not significantly different than anti-PD-1. Although H. acetispora is not significantly different than anti-PD-1, the data distribution is much tighter, and average tumor volume is smaller for H. acetispora smEV. The Tumor Growth Curve in FIG. 10 shows sustained efficacy of H. acetispora smEV treatment groups over 14 days of treatment similar to anti-PD-1.

### Third Representative Oncology Study

Female 8 week old BALB/c mice were acquired from Taconic Biosciences and allowed to acclimate at a vivarium for 1 week. On Day 0, mice were anesthetized with isoflurane, and inoculated subcutaneously on the left flank with 1.0e5 CT-26 cells (0.1 mL) prepared in PBS and Corning (GFR) Phenol Red-Free Matrigel (1:1). Mice were allowed to rest for 9 days post CT-26 inoculation to allow formation of palpable tumors. On Day 9, tumors were measured using a sliding digital caliper to collect length and width in measurements (in millimeters) to calculate estimated tumor volume ((L×W×W)/2)=TVmm3)). Mice were randomized into different treatment groups with a total of 9 mice per group. Randomization was done to balance all treatment groups, allowing each group to begin treatment with a similar average tumor volume and standard deviation. Dosing began on Day 10, and ended on Day 21 for 12 consecutive days of dosing. Mice were orally dosed QD and BID respectively with H. acetispora Strain A smEVs, or Q4D intraperitoneally with 200 ug anti-mouse PD-1 antibody. Body weight and tumor measurements were collected on a MWF schedule. Dose of smEVs was determined by particle count by NTA.

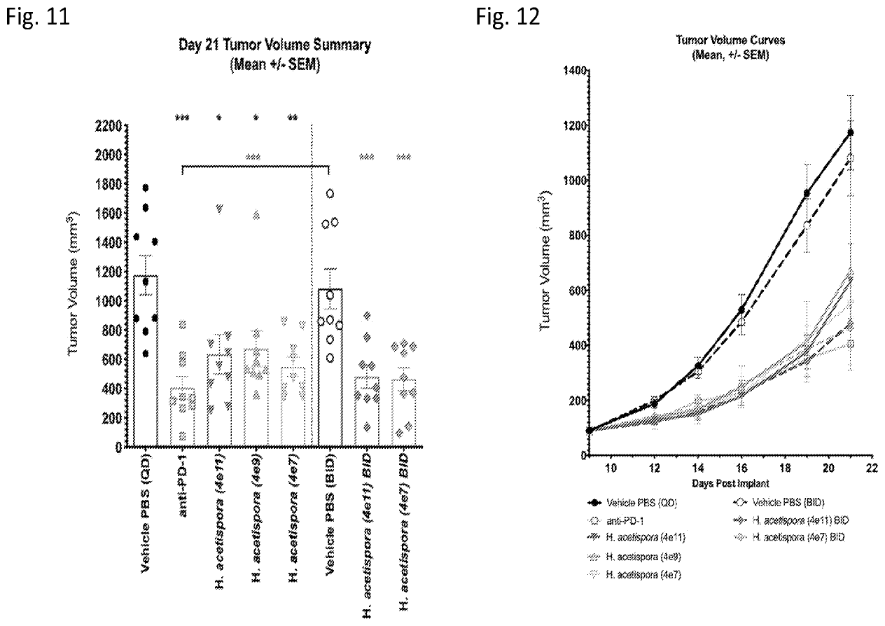

The Day 21 Tumor Volume Summary in FIG. 11 on the left, compares H. acetispora smEVs at 3 doses (4e11, 4e9, & 4e7), administered QD for 12 days against respective negative control (Vehicle PBS) and positive control (anti-PD-1). The Tumor Volume Summary in FIG. 11 also compares H. acetispora smEVs at 2 doses (2e11 & 2e7) administered BID for a total daily dose of 4e1 & 4e7 respectively against their corresponding negative control (Vehicle PBS BID) and positive control (anti-PD-1). The QD H. acetispora smEVs treatment arms compared to Vehicle PBS show statistically significant efficacy across all (3) doses, without an evident dose effect. The BID H. acetispora smEVs treatment arms compared to Vehicle PBS BID show significant efficacy, also no evidence of a dose effect. This result indicates both doses BID 2e11 & 2e7 are comparable dosed BID and QD. The Tumor Growth Curve in FIG. 12 shows a sustained efficacy after 12 days of dosing similar to anti-PD-1.

### Example 4: Fournierella Massillensis Strain A smEVs in a Mouse Model of Delayed-Type Hypersensitivity (DTH)

Delayed-type hypersensitivity (DTH) is an animal model of atopic dermatitis (or allergic contact dermatitis), as reviewed by Petersen et al. (In vivo pharmacological disease models for psoriasis and atopic dermatitis in drug discovery. Basic & Clinical Pharm & Toxicology. 2006, 99(2): 104-115; see also Irving C. Allen (ed.) Mouse Models of Innate Immunity: Methods and Protocols, Methods in Molecular Biology, 2013, vol. 1031, DOI 10.1007/978-1-62703-481-4_13). Several variations of the DTH model have been used and are well known in the art (Irving C. Allen (ed.). Mouse Models of Innate Immunity Methods and Protocols, Methods in Molecular Biology. Vol. 1031, DOI 10.1007/978-1-62703-481-4_13, Springer Science+Business Media, LLC 2013).

### First KLH DTH Study:

Female 5 week old C57BL/6 mice were purchased from Taconic Biosciences and acclimated at a vivarium for one week. Mice were primed with an emulsion of Keyhole Limpet Hemocyanin (KLH) and Complete Freund's Adjuvant (CFA) (1:1) by subcutaneous immunization on day 0. Mice were orally gavaged daily with smEVs or dosed intraperitoneally with dexamethasone at 1 mg/kg from days 1-8. After dosing on day 8, mice were anaesthetized with isoflurane, left ears were measured for baseline measurements with Fowler calipers and the mice were challenged intradermally with KLH in saline (10 μl) in the left ear and ear thickness measurements were taken at 24 hours. Dose was determined by particle count by NTA.

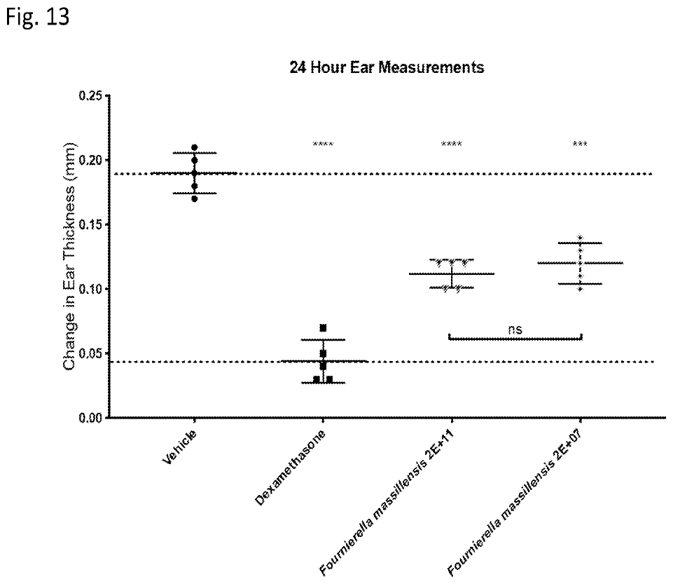

The 24 hour ear measurement results are shown in FIG. 13. smEVs made from Fournierella massillensis Strain A were compared at two doses, 2E+11 and 2E+07 (based on particles per dose). The smEVs were efficacious, showing decreased ear inflammation 24 hours after challenge.

### Example 5: Harryflintia Acetispora Strain A smEVs in a Mouse Model of Delayed-Type Hypersensitivity (DTH)

Delayed-type hypersensitivity (DTH) is an animal model of atopic dermatitis (or allergic contact dermatitis), as reviewed by Petersen et al. (In vivo pharmacological disease models for psoriasis and atopic dermatitis in drug discovery. Basic & Clinical Pharm & Toxicology. 2006, 99(2): 104-115; see also Irving C. Allen (ed.) Mouse Models of Innate Immunity: Methods and Protocols, Methods in Molecular Biology, 2013, vol. 1031, DOI 10.1007/978-1-62703-481-4_13). Several variations of the DTH model have been used and are well known in the art (Irving C. Allen (ed.). Mouse Models of Innate Immunity: Methods and Protocols, Methods in Molecular Biology. Vol. 1031, DOI 10.1007/978-1-62703-481-4_13, Springer Science+Business Media, LLC 2013).

### First KLH DTH Study:

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were orally gavaged daily with smEVs or dosed intraperitoneally with dexamethasone at 1 mg/kg from days 1-8. After dosing on day 8, mice were anaesthetized with isoflurane, left ears were measured for baseline measurements with Fowler calipers and the mice were challenged intradermally with KLH in saline (10 μl) in the left ear and ear thickness measurements were taken at 24 hours. Dose was determined by particle count by NTA.

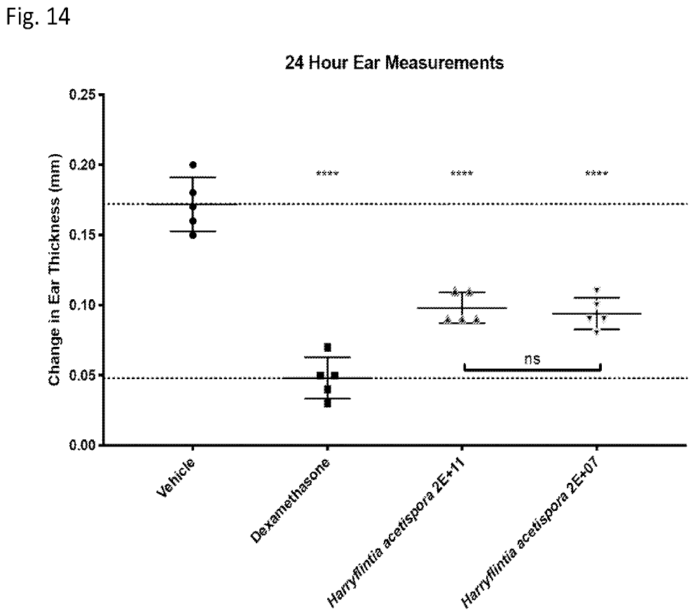

The 24 hour ear measurement results are shown in FIG. 14. smEVs made from Harryflintia acetispora Strain A were tested at two doses, 2E+11 and 2E+07, based on particles per dose. The smEVs were efficacious, showing decreased levels of ear inflammation 24 hours after challenge. Harryflintia acetispora Strain A was as efficacious at the lower dose as the higher dose.

### Example 6: Harryflintia acetispora Strain A smEVs in a Mouse Model of Delayed-Type Hypersensitivity (DTH)

Delayed-type hypersensitivity (DTH) is an animal model of atopic dermatitis (or allergic contact dermatitis), as reviewed by Petersen et al. (In vivo pharmacological disease models for psoriasis and atopic dermatitis in drug discovery. Basic & Clinical Pharm & Toxicology. 2006, 99(2): 104-115; see also Irving C. Allen (ed.) Mouse Models of Innate Immunity: Methods and Protocols. Methods in Molecular Biology, 2013, vol. 1031, DOI 10.1007/978-1-62703-481-4_13). Several variations of the DTH model have been used and are well known in the art (Irving C. Allen (ed.) Mouse Models of Innate Immunity: Methods and Protocols, Methods in Molecular Biology. Vol. 1031. DO1 10.1007/978-1-62703-481-4_13, Springer Science+Business Media, LLC 2013).

### KLH DTH Study:

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were orally gavaged daily with Harryflintia acetispora Strain A smEVs or dosed intraperitoneally with dexamethasone at 1 mg/kg from days 5-8. After dosing on day 8, mice were anaesthetized with isoflurane, left ears were measured for baseline measurements with Fowler calipers and the mice were challenged intradermally with KLH in saline (10 μl) in the left car and ear thickness measurements were taken at 24 hours.

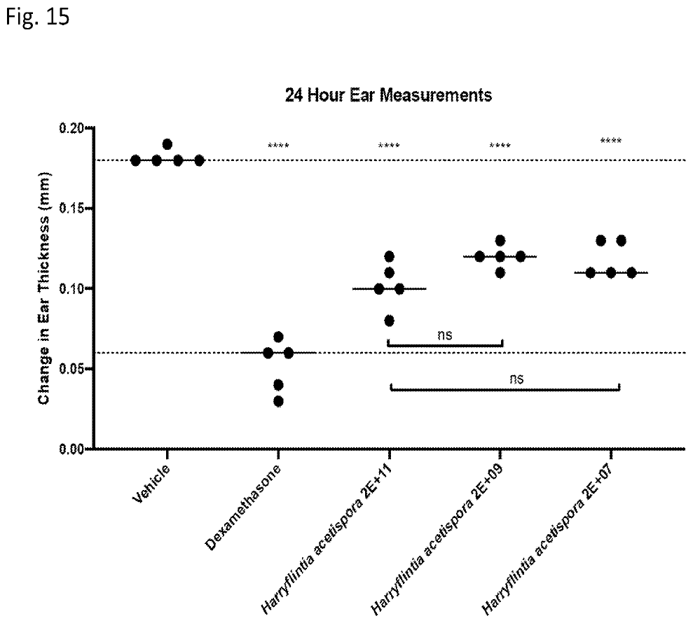

The 24 hour ear measurement results are shown in FIG. 15. smEVs made from Harryflintia acetispora Strain A were compared at three doses, 2E+11, 2E+09 and 2E+07 (based on particles per dose). The smEVs were efficacious at all three doses, showing decreased ear inflammation 24 hours after challenge.

### Example 7: Faecalibacterium prausnitzii Strain A smEVs in a Mouse Model of Delayed-Type Hypersensitivity (DTH)

Delayed-type hypersensitivity (DTH) is an animal model of atopic dermatitis (or allergic contact dermatitis), as reviewed by Petersen et al. (In vivo pharmacological disease models for psoriasis and atopic dermatitis in drug discovery. Basic & Clinical Pharm & Toxicology. 2006, 99(2): 104-115; see also Irving C. Allen (ed.) Mouse Models of Innate Immunity: Methods and Protocols. Methods in Molecular Biology, 2013, vol. 1031. DOI 10.1007/978-1-62703-481-4_13). Several variations of the DTH model have been used and are well known in the art (Irving C. Allen (ed.). Mouse Models of Innate Immunity: Methods and Protocols, Methods in Molecular Biology. Vol. 1031, DOI 10.1007/978-1-62703-481-4_13, Springer Science+Business Media, LLC 2013).

### First KLH DTH Study:

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were orally gavaged daily with smEVs or dosed intraperitoneally with dexamethasone at 1 mg/kg from days 1-8. After dosing on day 8, mice were anaesthetized with isoflurane, left ears were measured for baseline measurements with Fowler calipers and the mice were challenged intradermally with KLH in saline (10 μl) in the left ear and ear thickness measurements were taken at 24 hours. Dose was determined by particle count by NTA.

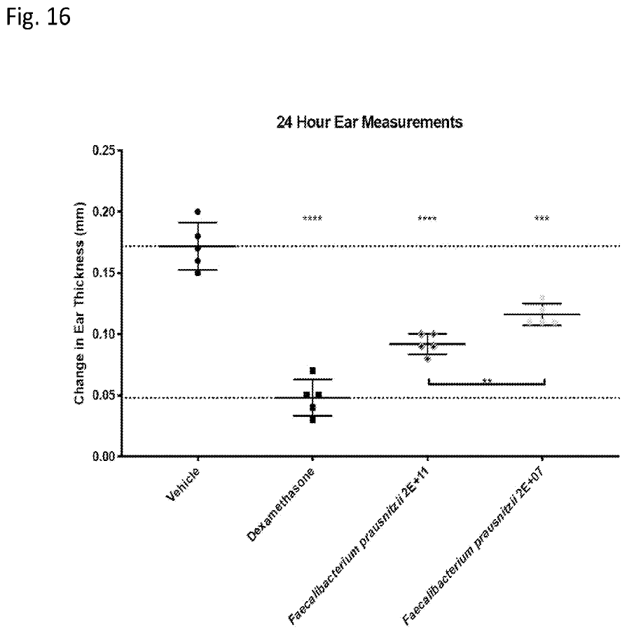

The 24 hour ear measurement results are shown in FIG. 16. smEVs made from Faecalibacterium prausnitzii Strain A were tested at two doses, 2E+11 and 2E+07, based on particles per dose. Both of these smEVs were efficacious, showing decreased levels of ear inflammation 24 hours after challenge. Faecalibacterium prausnitzii Strain A shows a dose response between the high and low doses.

### Example 8: Faecalibacterium prausnitzii Strain A smEVs in a Mouse Model of Delayed-Type Hypersensitivity (DTH)

Delayed-type hypersensitivity (DTH) is an animal model of atopic dermatitis (or allergic contact dermatitis), as reviewed by Petersen et al. (In vivo pharmacological disease models for psoriasis and atopic dermatitis in drug discovery. Basic & Clinical Pharm & Toxicology. 2006, 99(2): 104-115; see also Irving C. Allen (ed.) Mouse Models of Innate Immunity: Methods and Protocols. Methods in Molecular Biology, 2013, vol. 1031. DOI 10.1007/978-1-62703-481-4_13). Several variations of the DTH model have been used and are well known in the art (Irving C Allen (ed.). Mouse Models of Innate Immunity: Methods and Protocols, Methods in Molecular Biology. Vol. 1031, DOI 10.1007/978-1-62703-481-4_13, Springer Science+Business Media, LLC 2013).

### KLH DTH Study:

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were orally gavaged daily with Faecalibacterium prausnitzii Strain A smEVs or dosed intraperitoneally with dexamethasone at 1 mg/kg from days 5-8. After dosing on day 8, mice were anaesthetized with isoflurane, left ears were measured for baseline measurements with Fowler calipers and the mice were challenged intradermally with KLH in saline (10 μl) in the left ear and ear thickness measurements were taken at 24 hours.

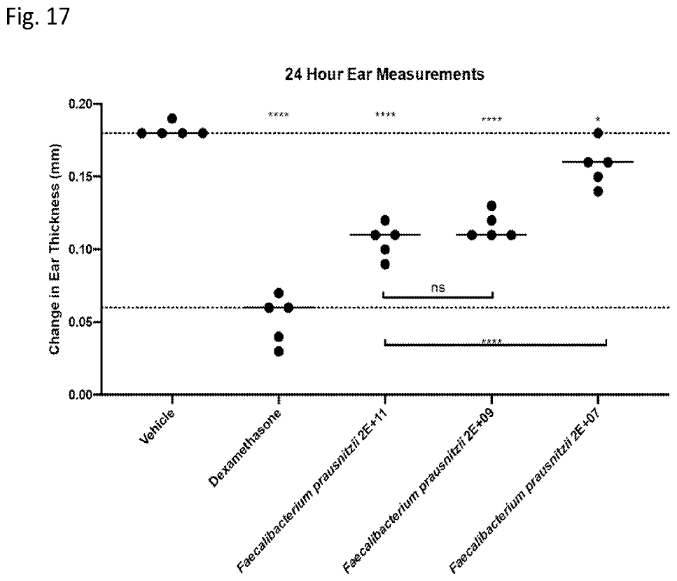

The 24 hour car measurement results are shown in FIG. 17. smEVs made from Faecalibacterium prausnitzii Strain A were compared at three doses, 2E+11, 2E+09 and 2E+07 (based on particles per dose). The smEVs were efficacious, showing decreased car inflammation 24 hours after challenge. The smEVs made from Faecalibacterium prausnitzii showed similar efficacy at the two highest doses and then lost some efficacy in the lowest dose.

### Example 9: smEV Isolation and Enumeration

### Equipment Required:

Sorvall RC-5C centrifuge with SLA-3000 rotor
Optima XE-90 Ultracentrifuge by Beckman-Coulter
   45Ti rotor
Sorvall wX+ Ultra Series Centrifuge by Thermo Scientific
   Fiberlite F37L-8×100 rotor
1. Microbial Supernatant Collection and Filtration:
   Microbes must be pelleted and filtered away from supernatant in order to recover smEVs and not microbes.
   a. Pellet Microbial culture
      i. Use Sorvall RC-5C centrifuge with the SLA-3000 rotor and centrifuge culture for a minimum of 15 min at a minimum of 7,000 rpm.
      ii. Decant supernatant into new and sterile container.
   b Supernatant Filtration
      i. Filter supernatant through 0.2 um filter.
      ii. For supernatants with poor filterability (less than 300 ml of supernatant pass through filter) attach a 0.45 um capsule filter ahead of the 0.2 um vacuum filter.
      iii. Store ‘filtered’ supernatant at 4° C.
      iv. Filtered supernatant can then be concentrated using TFF.
2. Isolation of smEVs using Ultracentrifugation
   Centrifuging concentrated supernatant in the ultracentrifuge will pellet smEVs isolating the smEVs from smaller biomolecules.
      i. Set speed for 200,000×g, time for 1 hour, and temperature at 4° C.
      ii. When rotor has stopped, remove tubes from ultracentrifuge and gently pour off the supernatant
      iii. Add more supernatant, balance, and centrifuge tubes again.
      iv. After all concentrated supernatant has been centrifuged, the pellets generated are referred to as ‘crude’ smEV pellets.
      v. Add sterile 1×PBS to pellets and place in container. Place on shaker, speed 70, in 4° C. fridge overnight or longer.
      vi. Resuspend the smEV pellets with additional sterile 1×PBS.
         1. Store resuspended crude smEV samples at 4° C. or at −80° C.
3. smEV Purification using Density Gradients

Density gradients are used for smEV purification. During ultracentrifugation, particles in the sample will move, and separate, within the graded density medium based on their ‘buoyant’ densities. In this way smEVs are separated from other particles, such as sugars, lipids, or other proteins, in the sample.
a. Preparation of Density Medium
   i. For smEV purification, four different percentages of the density medium (60% Optiprep) are used, a 45% layer, a 35% layer, a 25%, and a 15% layer. This will create the graded layers. A 0% layer is added at the top consisting of sterile 1×PBS.
   ii. The 45% gradient layer should contain the crude smEV sample. 5 ml of sample is added to 15 ml of Optiprep. If crude smEV sample is less than 5 ml, bring up to volume using sterile 1×PBS.
b Density Gradient Assembly
   i. Using a serological pipette, gently pipette the 45% gradient mixture up and down to mix. Then pipette the sample into a labeled clean and sterile ultracentrifuge tube.
   ii. Next, using a 10 ml serological pipette, slowly add 13 ml of 35% gradient mixture.
   iii. Continue with 13 ml of the 25% gradient mixture followed by 13 ml of the 15% mixture and finally 6 ml of sterile 1×PBS.
   iv. Balance ultracentrifuge tubes with sterile 1×PBS.
   v. Carefully place gradients in rotor and set the ultracentrifuge for 200.000×g and the temperature for 4° C. Centrifuge a minimum of 16 hours.
c. Removing Purified smEVs from Density Gradients
   i. Using a clean pipette, removed fraction(s) of interest and add to 15 ml conical tube.
   ii. Keep ‘purified’ smEV samples at 4° C.
d. Removing Optiprep Material from Purified smEVs
   i. In order to clean and remove residual optiprep from smEVs, Ox volume of PBS should be added to purified smEVs.
   ii. Set the ultracentrifuge for 200,000×g and the temperature for 4° C. Centrifuge for 1 hour.
   iii. Carefully remove tubes from ultracentrifuge and decant supernatant.
   iv. Continue ‘washing’ purified smEVs until all sample has been pelleted.
   v. Add sterile 1×PBS to purified pellets and place in container. Place on shaker, speed 70, in 4° C. fridge overnight or longer.
   vi. Resuspend the ‘purified’ smEV pellets with additional sterile 1×PBS.
      1. Store resuspended purified smEV samples at 4° C. or at −80° C.

### Example 10: Fournierella massiliensis Strain A smEV U937 Testing Protocol

### Cell Line Preparation

1. The U937 Monocyte cell line (ATCC) was propagated in RPMI medium with added FBS HEPES, sodium pyruvate, and antibiotic, at 37° C. with 5% CO₂.
2. Cells were enumerated using a cellometer with live/dead staining to determine viability.
3. Cells were diluted to a concentration of 5×10⁵ cells per ml in RPMI medium with 20 nM phorbol-12-myristate-13-acetate (PMA) to differentiate the monocytes into macrophage-like cells.
4. 200 microliters of cell suspension was added to each well of a 96-well plate and incubated 37° C. with 5% CO₂ for 72 hrs.
5. The adherent, differentiated cells were washed and incubated in fresh medium without PMA for 24 hrs before experimentation.

### Experimental Setup

1. smEVs were diluted to the appropriate concentration in RPMI medium without antibiotics (typically 1×10⁵-1×10¹⁰).
2. Treatment-free and TLR 2 and 4 agonist control samples are also prepared
3. The 96-well plate containing the differentiated U937 cells was washed with fresh RPMI medium without antibiotics, to remove residual antibiotics.
4. The suspension of smEVs was added to the washed plate.
5. The plate was incubated for 24 hrs at 37° C. with 5% CO₂.

### Experimental Endpoints

1. After 24 hrs of coincubation the supernatants were removed from the U937 cells into a separate 96-well plate. The cells were observed for any obvious lysis (plaques) in the wells.
2. Two treatment-free wells did not have the supernatants removed and Lysis buffer was added to the wells and incubated at 37° C. for 30 minutes to lyse cells (maximum lysis control).
3. 50 microliters of each supernatant or maximum lysis control was added to a new 96-well plate and cell lysis was determined (CytoTox 96® Non-Radioactive Cytotoxicity Assay, Promega) per manufacturer's instructions.
4. Cytokines were measured from the supernatants using U-plex MSD plates (Meso Scale Discovery) per manufacturer's instructions.

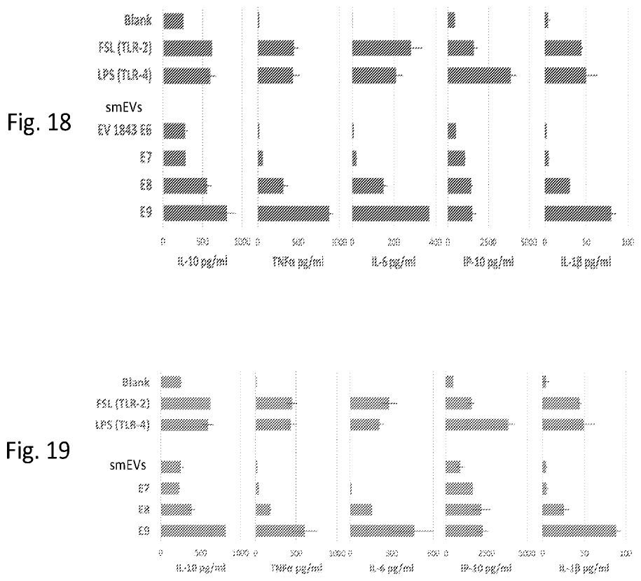

smEVs from Fournierella massiliensis Strain A induce cytokine production from PMA-differentiated 1937 cells (FIG. 18). U937 cells were treated with Fournierella massiliensis Strain A smEV at 1×10⁶-1×10⁹ concentrations as well as TLR2 (FSL) and TLR4 (LPS) agonist controls for 24 hrs and cytokine production was measured. Note the strong effect seen at low concentrations of smEVs from this strain. “Blank” indicates the medium control.

### Example 11: Harryflintia acetispora Strain A smEV U937 Testing Protocol

### Cell Line Preparation

1. The U937 Monocyte cell line (ATCC) was propagated in RPMI medium with added FBS HEPES, sodium pyruvate, and antibiotic, at 37° C. with 5% CO₂.
2. Cells were enumerated using a cellometer with live/dead staining to determine viability.
3. Cells were diluted to a concentration of 5×10⁵ cells per ml in RPMI medium with 20 nM phorbol-12-myristate-13-acetate (PMA) to differentiate the monocytes into macrophage-like cells.
4. 200 microliters of cell suspension was added to each well of a 96-well plate and incubated 37° C. with 5% CO₂ for 72 hrs.
5. The adherent, differentiated cells were washed and incubated in fresh medium without PMA for 24 hrs before experimentation.

### Experimental Setup

1. smEVs were diluted to the appropriate concentration in RPMI medium without antibiotics (typically 1×10⁵-1×10¹⁰).
2. Treatment-free and TLR 2 and 4 agonist control samples are also prepared
3. The 96-well plate containing the differentiated U937 cells was washed with fresh RPMI medium without antibiotics, to remove residual antibiotics.
4. The suspension of smEVs was added to the washed plate.
5. The plate was incubated for 24 hrs at 37° C. with 5% CO₂.

### Experimental Endpoints

1. After 24 hrs of coincubation the supernatants were removed from the U937 cells into a separate 96-well plate. The cells were observed for any obvious lysis (plaques) in the wells.
2 Two treatment-free wells did not have the supernatants removed and Lysis buffer was added to the wells and incubated at 37° C. for 30 minutes to lyse cells (maximum lysis control).
3. 50 microliters of each supernatant or maximum lysis control was added to a new 96-well plate and cell lysis was determined (CytoTox 96® Non-Radioactive Cytotoxicity Assay, Promega) per manufacturer's instructions.
4. Cytokines were measured from the supernatants using U-plex MSD plates (Meso Scale Discovery) per manufacturer's instructions

smEVs from Harryflintia acetispora Strain A induce cytokine production from PMA-differentiated U937 cells (FIG. 19). U937 cells were treated with Harryflintia acetispora Strain A smEV at 1×10⁶-1×10⁹ concentrations as well as TLR2 (FSL) and TLR4 (LPS) agonist controls for 24 hrs and cytokine production was measured. Note the stepwise increase in cytokine production. “Blank” indicates the medium control.

### Example 12: Faecalibacterium prausnitzii Strain A smEV U937 Testing Protocol

### Cell Line Preparation

1. The U937 Monocyte cell line (ATCC) was propagated in RPMI medium with added FBS HEPES, sodium pyruvate, and antibiotic, at 37° C. with 5% CO₂.
2. Cells were enumerated using a cellometer with live/dead staining to determine viability.
3. Cells were diluted to a concentration of 5×10⁵ cells per ml in RPMI medium with 20 nM phorbol-12-myristate-13-acetate (PMA) to differentiate the monocytes into macrophage-like cells.
4. 200 microliters of cell suspension was added to each well of a 96-well plate and incubated 37° C. with 5% CO₂ for 72 hrs.
5. The adherent, differentiated cells were washed and incubated in fresh medium without PMA for 24 hrs before experimentation.

### Experimental Setup

1. smEVs were diluted to the appropriate concentration in RPMI medium without antibiotics (typically 1×10⁵-1×10¹⁰).
2. Treatment-free and TLR 2 and 4 agonist control samples are also prepared
3. The 96-well plate containing the differentiated U937 cells was washed with fresh RPMI medium without antibiotics, to remove residual antibiotics
4. The suspension of smEVs was added to the washed plate.
5. The plate was incubated for 24 hrs at 37° C. with 5% CO₂.

### Experimental Endpoints

1. After 24 hrs of coincubation the supernatants were removed from the U937 cells into a separate 96-well plate. The cells were observed for any obvious lysis (plaques) in the wells.
2. Two treatment-free wells did not have the supernatants removed and Lysis buffer was added to the wells and incubated at 37° C. for 30 minutes to lyse cells (maximum lysis control).
3. 50 microliters of each supernatant or maximum lysis control was added to a new 96-well plate and cell lysis was determined (CytoTox 96® Non-Radioactive Cytotoxicity Assay, Promega) per manufacturer's instructions.
4. Cytokines were measured from the supernatants using U-plex MSD plates (Meso Scale Discovery) per manufacturer's instructions.

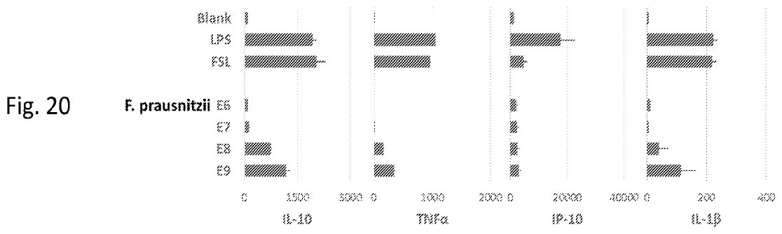

smEVs from Faecalibacterium prausnitzii Strain A induce cytokine production from PMA-differentiated U937 cells (FIG. 20). U937 cells were treated with Faecalibacterium prausnitzii Strain A smEVs at 1×10⁶-1×10⁹ concentrations as well as TLR2 (FSL) and TLR4 (LPS) agonist controls for 24 hrs and cytokine production was measured. Note the strong effect seen at low concentrations of smEVs from this strain. “Blank” indicates the medium control.

### Example 13: Administering pmEV Compositions to Treat Mouse Tumor Models

As described in Example 2, a mouse model of cancer is generated by subcutaneously injecting a tumor cell line or patient-derived tumor sample and allowing it to engraft into healthy mice. The methods provided herein may be performed using one of several different tumor cell lines including, but not limited to: B6-F10 or B16-F10-S1Y cells as an orthotopic model of melanoma, Panc02 cells as an orthotopic model of pancreatic cancer (Maletzki et al., 2008, Gut 57:483-491), LLC1 cells as an orthotopic model of lung cancer, and RM-1 as an orthotopic model of prostate cancer. As an example, but without limitation, methods for studying the efficacy of pmEVs in the B16-F10 model are provided in depth herein.

A syngeneic mouse model of spontaneous melanoma with a very high metastatic frequency is used to test the ability of bacteria to reduce tumor growth and the spread of metastases. The pmEVs chosen for this assay are compositions that may display enhanced activation of immune cell subsets and stimulate enhanced killing of tumor cells in vitro. The mouse melanoma cell line B16-F10 is obtained from ATCC. The cells are cultured in vitro as a monolayer in RPMI medium, supplemented with 10% heat-inactivated fetal bovine serum and 1% penicillin/streptomycin at 37° C. in an atmosphere of 5% C02 in air. The exponentially growing tumor cells are harvested by trypsinization, washed three times with cold 1×PBS, and a suspension of 5E6 cells/ml is prepared for administration. Female C57BL/6 mice are used for this experiment. The mice are 6-8 weeks old and weigh approximately 16-20 g. For tumor development, each mouse is injected SC into the flank with 100 μl of the B16-F10 cell suspension. The mice are anesthetized by ketamine and xylazine prior to the cell transplantation. The animals used in the experiment may be started on an antibiotic treatment via instillation of a cocktail of kanamycin (0.4 mg/ml), gentamicin, (0.035 mg/ml), colistin (850 U/ml), metronidazole (0.215 mg/ml) and vancomycin (0.045 mg/ml) in the drinking water from day 2 to 5 and an intraperitoneal injection of clindamycin (10 mg/kg) on day 7 after tumor injection.

The size of the primary flank tumor is measured with a caliper every 2-3 days and the tumor volume is calculated using the following formula: tumor volume=the tumor width×tumor length−0.5. After the primary tumor reaches approximately 100 mm3, the animals are sorted into several groups based on their body weight. The mice are then randomly taken from each group and assigned to a treatment group. pmEV compositions are prepared as previously described. The mice are orally inoculated by gavage with approximately 7.0e+09 to 3.0e+12 pmEV particles. Alternatively, pmEVs are administered intravenously. Mice receive pmEVs daily, weekly, bi-weekly, monthly, bi-monthly, or on any other dosing schedule throughout the treatment period. Mice may be IV injected with pmEVs in the tail vein, or directly injected into the tumor. Mice can be injected with pmEVs, with or without live bacteria, and/or pmEVs with or without inactivated/weakened or killed bacteria. Mice can be injected or orally gavaged weekly or once a month. Mice may receive combinations of purified pmEVs and live bacteria to maximize tumor-killing potential. All mice are housed under specific pathogen-free conditions following approved protocols. Tumor size, mouse weight, and body temperature are monitored every 3-4 days and the mice are humanely sacrificed 6 weeks after the B16-F10 mouse melanoma cell injection or when the volume of the primary tumor reaches 1000 mm3. Blood draws are taken weekly and a full necropsy under sterile conditions is performed at the termination of the protocol.

Cancer cells can be easily visualized in the mouse B16-F10 melanoma model due to their melanin production. Following standard protocols, tissue samples from lymph nodes and organs from the neck and chest region are collected and the presence of micro- and macro-metastases is analyzed using the following classification rule. An organ is classified as positive for metastasis if at least two micro-metastatic and one macro-metastatic lesion per lymph node or organ are found. Micro-metastases are detected by staining the paraffin-embedded lymphoid tissue sections with hematoxylin-eosin following standard protocols known to one skilled in the art. The total number of metastases is correlated to the volume of the primary tumor and it is found that the tumor volume correlates significantly with tumor growth time and the number of macro- and micro-metastases in lymph nodes and visceral organs and also with the sum of all observed metastases. Twenty-five different metastatic sites are identified as previously described (Bobek V., et al., Syngeneic lymph-node-targeting model of green fluorescent protein-expressing Lewis lung carcinoma, Clin. Exp. Metastasis, 2004; 21(8):705-8).

The tumor tissue samples are further analyzed for tumor infiltrating lymphocytes. The CD8+ cytotoxic T cells can be isolated by FACS and can then be further analyzed using customized p/MHC class I microarrays to reveal their antigen specificity (see e.g., Deviren G., et al., Detection of antigen-specific T cells on p/MHC microarrays, J. Mol. Recognit., 2007 January-February; 20(1):32-8). CD4+ T cells can be analyzed using customized p/MHC class II microarrays.

At various timepoints, mice are sacrificed and tumors, lymph nodes, or other tissues may be removed for ex vivo flow cytometric analysis using methods known in the art. For example, tumors are dissociated using a Miltenyi tumor dissociation enzyme cocktail according to the manufacturer's instructions. Tumor weights are recorded and tumors are chopped then placed in 15 ml tubes containing the enzyme cocktail and placed on ice. Samples are then placed on a gentle shaker at 37° C. for 45 minutes and quenched with up to 15 ml complete RPMI. Each cell suspension is strained through a 70 μm filter into a 50 ml falcon tube and centrifuged at 1000 rpm for 10 minutes. Cells are resuspended in FACS buffer and washed to remove remaining debris. If necessary, samples are strained again through a second 70 μm filter into a new tube. Cells are stained for analysis by flow cytometry using techniques known in the art. Staining antibodies can include anti-CD11c (dendritic cells), anti-CD80, anti-CD86, anti-CD40, anti-MHCII, anti-CD8a, anti-CD4, and anti-CD103. Other markers that may be analyzed include pan-immune cell marker CD45, T cell markers (CD3, CD4, CD8, CD25, Foxp3, T-bet, Gata3, Rorγt, Granzyme B, CD69, PD-1. CTLA-4), and macrophage/mycloid markers (CD11b, MHCII, CD206, CD40, CSF1R, PD-L1, Gr-1). In addition to immunophenotyping, serum cytokines can be analyzed including, but not limited to, TNFa, IL-17, IL-13, IL-12p70, IL12p40, IL-10, IL-6, IL-5, IL-4, IL-2, IL-1b, IFNy, GM-CSF, G-CSF, M-CSF, MIG, IP10, MIP1b, RANTES, and MCP-1. Cytokine analysis may be carried out immune cells obtained from lymph nodes or other tissue, and/or on purified CD45+ tumor-infiltrated immune cells obtained ex vivo. Finally, immunohistochemistry is carried out on tumor sections to measure T cells, macrophages, dendritic cells, and checkpoint molecule protein expression.

The same experiment is also performed with a mouse model of multiple pulmonary melanoma metastases. The mouse melanoma cell line B16-BL6 is obtained from ATCC and the cells are cultured in vitro as described above. Female C57BL/6 mice are used for this experiment. The mice are 6-8 weeks old and weigh approximately 16-20 g. For tumor development, each mouse is injected into the tail vein with 100 μl of a 2E6 cells/nil suspension of B16-BL6 cells. The tumor cells that engraft upon IV injection end up in the lungs.

The mice are humanely killed after 9 days. The lungs are weighed and analyzed for the presence of pulmonary nodules on the lung surface. The extracted lungs are bleached with Fekete's solution, which does not bleach the tumor nodules because of the melanin in the B16 cells though a small fraction of the nodules is amelanotic (i.e. white). The number of tumor nodules is carefully counted to determine the tumor burden in the mice. Typically, 200-250 pulmonary nodules are found on the lungs of the control group mice (i.e. PBS gavage).

The percentage tumor burden is calculated for the three treatment groups. Percentage tumor burden is defined as the mean number of pulmonary nodules on the lung surfaces of mice that belong to a treatment group divided by the mean number of pulmonary nodules on the lung surfaces of the control group mice.

The tumor biopsies and blood samples are submitted for metabolic analysis via LCMS techniques or other methods known in the art. Differential levels of amino acids, sugars, lactate, among other metabolites, between test groups demonstrate the ability of the microbial composition to disrupt the tumor metabolic state.

### RNA Seq to Determine Mechanism of Action

Dendritic cells are purified from tumors, Peyers patches, and mesenteric lymph nodes. RNAseq analysis is carried out and analyzed according to standard techniques known to one skilled in the art (Z. Hou. Scientific Reports. 5(9570):doi:10.1038/srep09570 (2015)). In the analysis, specific attention is placed on innate inflammatory pathway genes including TLRs, CLRs, NLRs, and STING, cytokines, chemokines, antigen processing and presentation pathways, cross presentation, and T cell co-stimulation.

Rather than being sacrificed, some mice may be rechallenged with tumor cell injection into the contralateral flank (or other area) to determine the impact of the immune system's memory response on tumor growth.

### Example 14: Administering pmEVs to Treat Mouse Tumor Models in Combination with PD-1 or PD-L1 Inhibition

To determine the efficacy of pmEVs in tumor mouse models, in combination with PD-1 or PD-L1 inhibition, a mouse tumor model may be used as described above.

pmEVs are tested for their efficacy in the mouse tumor model, either alone or in combination with whole bacterial cells and with or without anti-PD-1 or anti-PD-L1. pmEVs, bacterial cells, and/or anti-PD-1 or anti-PD-L1 are administered at varied time points and at varied doses. For example, on day 10 after tumor injection, or after the tumor volume reaches 100 mm³, the mice are treated with pmEVs alone or in combination with anti-PD-1 or anti-PD-L1.

Mice may be administered pmEVs orally, intravenously, or intratumorally. For example, some mice are intravenously injected with anywhere between 7.0c+09 to 3.0c+12 pmEV particles. While some mice receive pmEVs through i.v. injection, other mice may receive pmEVs through intraperitoneal (i.p.) injection, subcutaneous (s.c.) injection, nasal route administration, oral gavage, or other means of administration. Some mice may receive pmEVs every day (e.g., starting on day 1), while others may receive pmEVs at alternative intervals (e.g., every other day, or once every three days). Groups of mice may be administered a pharmaceutical composition of the invention comprising a mixture of pmEVs and bacterial cells. For example, the composition may comprise pmEV particles and whole bacteria in a ratio from 1:1 (pmEVs: bacterial cells) to 1×100¹²:1 (pmEVs: bacterial cells).

Alternatively, some groups of mice may receive between 1×10⁴ and 5×10⁹ bacterial cells in an administration separate from, or comingled with, the pmEV administration. As with the pmEVs, bacterial cell administration may be varied by route of administration, dose, and schedule. The bacterial cells may be live, dead, or weakened. The bacterial cells may be harvested fresh (or frozen) and administered, or they may be irradiated or heat-killed prior to administration with the pmEVs. Some groups of mice are also injected with effective doses of checkpoint inhibitor For example, mice receive 100 μg anti-PD-L1 mAB (clone 10f.9g2, BioXCell) or another anti-PD-1 or anti-PD-L1 mAB in 100 μl PBS, and some mice receive vehicle and/or other appropriate control (e.g., control antibody). Mice are injected with mABs 3, 6, and 9 days after the initial injection. To assess whether checkpoint inhibition and pmEV immunotherapy have an additive anti-tumor effect, control mice receiving anti-PD-1 or anti-PD-L1 mABs are included to the standard control panel. Primary (tumor size) and secondary (tumor infiltrating lymphocytes and cytokine analysis) endpoints are assessed, and some groups of mice may be rechallenged with a subsequent tumor cell inoculation to assess the effect of treatment on memory response.

### Example 15: Labeling Bacterial pmEVs

pmEVs may be labeled in order to track their biodistribution in vivo and to quantify and track cellular localization in various preparations and in assays conducted with mammalian cells. For example, pmEVs may be radio-labeled, incubated with dyes, fluorescently labeled, luminescently labeled, or labeled with conjugates containing metals and isotopes of metals.

For example, pmEVs may be incubated with dyes conjugated to functional groups such as NHS-ester, click-chemistry groups, streptavidin or biotin. The labeling reaction may occur at a variety of temperatures for minutes or hours, and with or without agitation or rotation. The reaction may then be stopped by adding a reagent such as bovine serum albumin (BSA), or similar agent, depending on the protocol, and free or unbound dye molecule removed by ultra-centrifugation, filtration, centrifugal filtration, column affinity purification or dialysis. Additional washing steps involving wash buffers and vortexing or agitation may be employed to ensure complete removal of free dyes molecules such as described in Su Chul Jang et al, Small. 11, No. 4, 456-461(2017).

Optionally, pmEVs may be concentrated to 5.0 E12 particle/ml (300 ug) and diluted up to 1.8 mo using 2× concentrated PBS buffer pH 8.2 and pelleted by centrifugation at 165,000×g at 4 C using a benchtop ultracentrifuge. The pellet is resuspended in 300 ul 2×PBS pH 8.2 and an NIS-ester fluorescent dye is added at a final concentration of 0.2 mM from a 10 mM dye stock (dissolved in DMSO). The sample is gently agitated at 24° C. for 1.5 hours, and then incubated overnight at 4° C. Free non-reacted dye is removed by 2 repeated steps of dilution/pelleting as described above, using 1×PBS buffer, and resuspending in 300 ul final volume.

Fluorescently labeled pmEVs are detected in cells or organs, or in in vitro and/or ex vivo samples by confocal microscopy, nanoparticle tracking analysis, flow cytometry, fluorescence activated cell sorting (FACs) or fluorescent imaging system such as the Odyssey CLx LICOR (see e.g., Wiklander et al. 2015. J. Extracellular Vesicles. 4:10 3402/jev.v4.26316). Additionally, fluorescently labeled pmEVs are detected in whole animals and/or dissected organs and tissues using an instrument such as the IVIS spectrum CT (Perkin Elmer) or Pearl Imager, as in H-I. Choi, et al. Experimental & Molecular Medicine. 49: e330 (2017).

pmEVs may be labeled with conjugates containing metals and isotopes of metals using the protocols described above. Metal-conjugated pmEVs may be administered in vivo to animals. Cells may then be harvested from organs at various time-points, and analyzed ex vivo. Alternatively, cells derived from animals, humans, or immortalized cell lines may be treated with metal-labelled pmEVs in vitro and cells subsequently labelled with metal-conjugated antibodies and phenotyped using a Cytometry by Time of Flight (CyTOF) instrument such as the Helios CyTOF (Fluidigm) or imaged and analyzed using and Imaging Mass Cytometry instrument such as the Hyperion Imaging System (Fluidigm). Additionally, pmEVs may be labelled with a radioisotope to track the pmEVs biodistribution (see, e.g., Miller et al., Nanoscale. 2014 May 7; 6(9):4928-35).

### Example 16: Transmission Electron Microscopy to Visualize Bacterial pmEVs

pmEVs are prepared from bacteria batch cultures. Transmission electron microscopy (TEM) may be used to visualize purified bacterial pmEVs (S. Bin Park, et al. PLoS ONE. 6(3):e17629 (2011). pmEVs are mounted onto 300- or 400-mesh-size carbon-coated copper grids (Electron Microscopy Sciences, USA) for 2 minutes and washed with deionized water. pmEVs are negatively stained using 2% (w/v) uranyl acetate for 20 sec-1 min. Copper grids are washed with sterile water and dried. Images are acquired using a transmission electron microscope with 100-120 kV acceleration voltage. Stained pmEVs appear between 20-600 nm in diameter and are electron dense. 10-50 fields on each grid are screened.

### Example 17: Profiling pmEV Composition and Content

pmEVs may be characterized by any one of various methods including, but not limited to, NanoSight characterization, SDS-PAGE gel electrophoresis, Western blot, ELISA, liquid chromatography-mass spectrometry and mass spectrometry, dynamic light scattering, lipid levels, total protein, lipid to protein ratios, nucleic acid analysis and/or zeta potential.

NanoSight Characterization of pmEVs

Nanoparticle tracking analysis (NTA) is used to characterize the size distribution of purified bacterial pmEVs. Purified pmEV preparations are run on a NanoSight machine (Malvern Instruments) to assess pmEV size and concentration.

### SDS-PAGE Gel Electrophoresis

To identify the protein components of purified pmEVs, samples are run on a gel, for example a Bolt Bis-Tris Plus 4-12% gel (Thermo-Fisher Scientific), using standard techniques. Samples are boiled in 1×SDS sample buffer for 10 minutes, cooled to 4° C., and then centrifuged at 16,000×g for 1 min. Samples are then run on a SDS-PAGE gel and stained using one of several standard techniques (e.g., Silver staining, Coomassie Blue, Gel Code Blue) for visualization of bands.

### Western Blot Analysis

To identify and quantify specific protein components of purified pmEVs, pmEV proteins are separated by SDS-PAGE as described above and subjected to Western blot analysis (Cvjetkovic et al., Sci. Rep. 6, 36338 (2016)) and are quantified via ELISA.

pmEV proteomics and Liquid Chromatography-Mass Spectrometry (LC-MS/MS) and Mass Spectrometry (MS)

Proteins present in pmEVs are identified and quantified by Mass Spectrometry techniques. pmEV proteins may be prepared for LC-MS/MS using standard techniques including protein reduction using dithiotreitol solution (DTT) and protein digestion using enzymes such as LysC and trypsin as described in Erickson et al, 2017 (Molecular Cell, VOLUME 65, ISSUE 2, P361-370, Jan. 19, 2017). Alternatively, peptides are prepared as described by Liu et al. 2010 (JOURNAL OF BACTERIOLOGY, June 2010, p. 2852-2860 Vol. 192, No 11), Kicselbach and Oscarsson 2017 (Data Brief. 2017 February; 10: 426-431), Vildhede et al, 2018 (Drug Metabolism and Disposition Feb. 8, 2018). Following digestion, peptide preparations are run directly on liquid chromatography and mass spectrometry devices for protein identification within a single sample. For relative quantitation of proteins between samples, peptide digests from different samples are labeled with isobaric tags using the iTRAQ Reagent-8plex Multiplex Kit (Applied Biosystems, Foster City, Calif.) or TMT 10plex and 11plex Label Reagents (Thermo Fischer Scientific, San Jose, Calif., USA). Each peptide digest is labeled with a different isobaric tag and then the labeled digests are combined into one sample mixture. The combined peptide mixture is analyzed by LC-MS/MS for both identification and quantification. A database search is performed using the LC-MS/MS data to identify the labeled peptides and the corresponding proteins. In the case of isobaric labeling, the fragmentation of the attached tag generates a low molecular mass reporter ion that is used to obtain a relative quantitation of the peptides and proteins present in each pmEV.

Additionally, metabolic content is ascertained using liquid chromatography techniques combined with mass spectrometry A variety of techniques exist to determine metabolomic content of various samples and are known to one skilled in the art involving solvent extraction, chromatographic separation and a variety of ionization techniques coupled to mass determination (Roberts et al 2012 Targeted Metabolomics. Curr Protoc Mol Biol. 30: 1-24: Dettmer et al 2007, Mass spectrometry-based metabolomics. Mass Spectrom Rev. 26(1):51-78). As a non-limiting example, a LC-MS system includes a 4000 QTRAP triple quadrupole mass spectrometer (AB SCIEX) combined with 1100 Series pump (Agilent) and an HTS PAL autosampler (Lcap Technologies). Media samples or other complex metabolic mixtures (˜10 μL) are extracted using nine volumes of 74.9:24.9:0.2 (v/v/v) acetonitrile/methanol/formic acid containing stable isotope-labeled internal standards (valine-d8, Isotec; and phenylalanine-d8, Cambridge Isotope Laboratories). Standards may be adjusted or modified depending on the metabolites of interest. The samples are centrifuged (10 minutes, 9,000×g, 4° C.), and the supernatants (10 μL) are submitted to LCMS by injecting the solution onto the HLIC column (150×2.1 mm, 3 μm particle size). The column is eluted by flowing a 5% mobile phase [10 mM ammonium formate, 0.1% formic acid in water] for 1 minute at a rate of 250 uL/minute followed by a linear gradient over 10 minutes to a solution of 40% mobile phase [acetonitrile with 0.1% formic acid]. The ion spray voltage is set to 4.5 kV and the source temperature is 450° C.

The data are analyzed using commercially available software like Multiquant 1.2 from AB SCIEX for mass spectrum peak integration. Peaks of interest should be manually curated and compared to standards to confirm the identity of the peak. Quantitation with appropriate standards is performed to determine the number of metabolites present in the initial media, after bacterial conditioning and after tumor cell growth A non-targeted metabolomics approach may also be used using metabolite databases, such as but not limited to the NIST database, for peak identification.

### Dynamic Light Scattering (DLS)

DLS measurements, including the distribution of particles of different sizes in different pmEV preparations are taken using instruments such as the DynaPro NanoStar (Wyatt Technology) and the Zetasizer Nano ZS (Malvern Instruments).

### Lipid Levels

Lipid levels are quantified using FM4-64 (Life Technologies), by methods similar to those described by A. J. McBroom et al. J Bacteriol 188:5385-5392, and A. Frias, et al. Microb Ecol. 59-476-486 (2010). Samples are incubated with FM4-64 (3.3 μg/mL in PBS for 10 minutes at 37° C. in the dark). After excitation at 515 nm, emission at 635 nm is measured using a Spectramax M5 plate reader (Molecular Devices). Absolute concentrations are determined by comparison of unknown samples to standards (such as palmitoyloleoylphosphatidylglycerol (POPG) vesicles) of known concentrations. Lipidomics can be used to identify the lipids present in the pmEVs.

### Total Protein

Protein levels are quantified by standard assays such as the Bradford and BCA assays. The Bradford assays are run using Quick Start Bradford 1× Dye Reagent (Bio-Rad), according to manufacturer's protocols. BCA assays are run using the Pierce BCA Protein Assay Kit (Thermo-Fisher Scientific). Absolute concentrations are determined by comparison to a standard curve generated from BSA of known concentrations. Alternatively, protein concentration can be calculated using the Beer-Lambert equation using the sample absorbance at 280 nm (A280) as measured on a Nanodrop spectrophotometer (Thermo-Fisher Scientific). In addition, proteomics may be used to identify proteins in the sample.

### Lipid:Protein Ratios

Lipid:protein ratios are generated by dividing lipid concentrations by protein concentrations. These provide a measure of the purity of vesicles as compared to free protein in each preparation.

### Nucleic Acid Analysis

Nucleic acids are extracted from pmEVs and quantified using a Qubit fluorometer. Size distribution is assessed using a BioAnalyzer and the material is sequenced.

### Zeta Potential

The zeta potential of different preparations are measured using instruments such as the Zetasizer ZS (Malvern Instruments).

### Example 18: In Vitro Screening of pmEVs for Enhanced Activation of CD8+ T Cell Killing when Incubated with Tumor Cells

In vitro methods for screening pmEVs that can activate CD8+ T cell killing of tumor cells are described. Briefly, DCs are isolated from human PBMCs or mouse spleens, using techniques known in the art, and incubated in vitro with single-strain pmEVs, mixtures of pmEVs, and/or appropriate controls. In addition, CD84+ T cells are obtained from human PBMCs or mouse spleens using techniques known in the art, for example the magnetic bead-based Mouse CD8a+ T Cell Isolation Kit and the magnetic bead-based Human CD8+ T Cell Isolation Kit (both from Miltenyi Biotech, Cambridge, Mass.). After incubation of DCs with pmEVs for some time (e.g., for 24-hours), or incubation of DCs with pmEV-stimulated epithelial cells, pmEVs are removed from the cell culture with PBS washes and 100 ul of fresh media with antibiotics is added to each well, and 200,000 T cells are added to each experimental well in the 96-well plate. Anti-CD3 antibody is added at a final concentration of 2 ug/ml. Co-cultures are then allowed to incubate at 37° C. for 96 hours under normal oxygen conditions.

For example, approximately 72 hours into the coculture incubation, tumor cells are plated for use in the assay using techniques known in the art. For example, 50,000 tumor cells/well are plated per well in new 96-well plates. Mouse tumor cell lines used may include B16.F10, SIY+B16.F10, and others. Human tumor cell lines are HLA-matched to donor, and can include PANC-1, UNKPC960/961, UNKC, and HELA cell lines. After completion of the 96-hour co-culture, 100 μl of the CD8+ T cell and DC mixture is transferred to wells containing tumor cells. Plates are incubated for 24 hours at 37° C. under normal oxygen conditions. Staurospaurine may be used as negative control to account for cell death.

Following this incubation, flow cytometry is used to measure tumor cell death and characterize immune cell phenotype. Briefly, tumor cells are stained with viability dye. FACS analysis is used to gate specifically on tumor cells and measure the percentage of dead (killed) tumor cells. Data are also displayed as the absolute number of dead tumor cells per well. Cytotoxic CD8+ T cell phenotype may be characterized by the following methods: a) concentration of supernatant granzyme B, IFNy and TNFa in the culture supernatant as described below. b) CD8+ T cell surface expression of activation markers such as DC69, CD25, CD154, PD-1, gamma/delta TCR, Foxp3, T-bet, granzyme B. c) intracellular cytokine staining of IFNy, granzyme B. TNFa in CD8+ T cells. CD4+ T cell phenotype may also be assessed by intracellular cytokine staining in addition to supernatant cytokine concentration including INFy, TNFa, IL-12, IL-4, IL-5, IL-17, IL-10, chemokines etc.

As an additional measure of CD8+ T cell activation, 100 μl of culture supernatant is removed from wells following the 96-hour incubation of T cells with DCs and is analyzed for secreted cytokines, chemokines, and growth factors using the multiplexed Luminex Magpix. Kit (EMD Millipore, Darmstadt, Germany). Briefly, the wells are pie-wet with buffer, and 25 μl of 1× antibody-coated magnetic beads are added and 2×200 μl of wash buffer are performed in every well using the magnet. 50 μl of Incubation buffer, 50 μl of diluent and 50 μl of samples am added and mixed via shaking for 2 hrs at room temperature in the dark. The beads are then washed twice with 200 μl wash buffer. 100 μl of 1× biotinylated detector antibody is added and the suspension is incubated for 1 hour with shaking in the dark. Two, 200 μl washes are then performed with wash buffer. 100 μl of 1×SAV-RPE reagent is added to each well and is incubated for 30 min at RI in the dark. Three 200 μl washes are performed and 125 μl of wash buffer is added with 2-3 min shaking occurs. The wells are then submitted for analysis in the Luminex xMAP system.

Standards allow for careful quantitation of the cytokines including GM-CSF, IFN-g, IFN-a, IFN-B IL-1a, IL-1B, IL-2, IL-4, IL-5, IL-6, IL-8, IL-10, IL-13, IL-12 (p40/p70), IL-17, IL-23, IP-10, KC, MCP-1, MIG, MIP1a, TNFa, and VEGF. These cytokines are assessed in samples of both mouse and human origin. Increases in these cytokines in the bacterial treated samples indicate enhanced production of proteins and cytokines from the host. Other variations on this assay examining specific cell types ability to release cytokines are assessed by acquiring these cells through sorting methods and are recognized by one of ordinary skill in the art. Furthermore, cytokine mRNA is also assessed to address cytokine release in response to an pmEV composition. These changes in the cells of the host stimulate an immune response similarly to in vivo response in a cancer microenvironment.

This CD8+ T cell stimulation protocol may be repeated using combinations of purified pmEVs and live bacterial strains to maximize immune stimulation potential.

### Example 19: In Vitro Screening of pmEVs for Enhanced Tumor Cell killing by PBMCs

Various methods may be used to screen pmEVs for the ability to stimulate PBMCs, which in turn activate CD8+ T cells to kill tumor cells. For example, PBMCs are isolated from heparinized venous blood from healthy human donors by ficoll-paque gradient centrifugation for mouse or human blood, or with Lympholyte Cell Separation Media (Cedarlane Labs, Ontario, Canada) from mouse blood. PBMCs are incubated with single-strain pmEVs, mixtures of pmEVs, and appropriate controls. In addition, CD8+ T cells are obtained from human PBMCs or mouse spleens. After the 24-hour incubation of PBMCs with pmEVs, pmEVs are removed from the cells using PBS washes. 100 μl of fresh media with antibiotics is added to each well. An appropriate number of T cells (e.g., 200,000 T cells) are added to each experimental well in the 96-well plate. Anti-CD3 antibody is added at a final concentration of 2 μg/ml. Co-cultures are then allowed to incubate at 37° C. for 96 hours under normal oxygen conditions.

For example, 72 hours into the coculture incubation, 50,000 tumor cells/well are plated per well in new 96-well plates. Mouse tumor cell lines used include B16.F10, SIY+B16.F10, and others. Human tumor cell lines are HLA-matched to donor, and can include PANC-1, UNKPC960/961, UNKC, and HELA cell lines. After completion of the 96-hour co-culture, 100 μl of the CD8+ T cell and PBMC mixture is transferred to wells containing tumor cells. Plates are incubated for 24 hours at 37° C. under normal oxygen conditions. Staurospaurine is used as negative control to account for cell death.

Following this incubation, flow cytometry is used to measure tumor cell death and characterize immune cell phenotype. Briefly, tumor cells are stained with viability dye. FACS analysis is used to gate specifically on tumor cells and measure the percentage of dead (killed) tumor cells. Data are also displayed as the absolute number of dead tumor cells per well. Cytotoxic CD8+ T cell phenotype may be characterized by the following methods: a) concentration of supernatant granzyme B, IFNy and TNFa in the culture supernatant as described below, b) CD8+ T cell surface expression of activation markers such as DC69, CD25, CD154, PD-1, gamma/delta TCR, Foxp3, T-bet, granzyme B, c) intracellular cytokine staining of IFNγ, granzyme B, TNFa in CD8++ T cells. CD4++ T cell phenotype may also be assessed by intracellular cytokine staining in addition to supernatant cytokine concentration including INFy, TNFa, IL-12, IL-4, IL-5, IL-17, IL-10, chemokines etc.

As an additional measure of CD8+ T cell activation, 100 μl of culture supernatant is removed from wells following the 96-hour incubation of T cells with DCs and is analyzed for secreted cytokines, chemokines, and growth factors using the multiplexed Luminex Magpix. Kit (EMD Millipore, Darmstadt, Germany). Briefly, the wells are pre-wet with buffer, and 25 μl of 1× antibody-coated magnetic beads are added and 2×200 μl of wash buffer are performed in every well using the magnet. 50 μl of Incubation buffer, 50 μl of diluent and 50 μl of samples are added and mixed via shaking for 2 hrs at room temperature in the dark. The beads are then washed twice with 200 μl wash buffer. 100 μl of 1× biotinylated detector antibody is added and the suspension is incubated for 1 hour with shaking in the dark. Two, 200 μl washes are then performed with wash buffer. 100 μl of 1×SAV-RPE reagent is added to each well and is incubated for 30 min at RT in the dark. Three 200 μl washes are performed and 125 μl of wash buffer is added with 2-3 min shaking occurs. The wells are then submitted for analysis in the Luminex xMAP system.

Standards allow for careful quantitation of the cytokines including GM-CSF, IFN-g, IFN-a, iFN-B IL-1a, IL-1B, IL-2, IL-4, IL-5, IL-6, IL-8, IL-10, IL-13, IL-12 (p40/p70), IL-17, IL-23, IP-10, KC, MCP-1, MIG, MIP1a, TNFα, and VEGF. These cytokines are assessed in samples of both mouse and human origin. Increases in these cytokines in the bacterial treated samples indicate enhanced production of proteins and cytokines from the host. Other variations on this assay examining specific cell types ability to release cytokines are assessed by acquiring these cells through sorting methods and are recognized by one of ordinary skill in the art. Furthermore, cytokine mRNA is also assessed to address cytokine release in response to an pmEV composition. These changes in the cells of the host stimulate an immune response similarly to in vivo response in a cancer microenvironment.

This PBMC stimulation protocol may be repeated using combinations of purified pmEVs with or without combinations of live, dead, or inactivated/weakened bacterial strains to maximize immune stimulation potential.

### Example 20: In Vitro Detection of pmEVs in Antigen-Presenting Cells

Dendritic cells in the lamina propria constantly sample live bacteria, dead bacteria, and microbial products in the gut lumen by extending their dendrites across the gut epithelium, which is one way that pmEVs produced by bacteria in the intestinal lumen may directly stimulate dendritic cells. The following methods represent a way to assess the differential uptake of pmEVs by antigen-presenting cells. Optionally, these methods may be applied to assess immunomodulatory behavior of pmEVs administered to a patient.

Dendritic cells (DCs) are isolated from human or mouse bone marrow, blood, or spleens according to standard methods or kit protocols (e.g., Inaba K, Swiggard W J, Steinman R M, Romani N, Schuler G, 2001. Isolation of dendritic cells. Current Protocols in Immunology. Chapter 3: Unit3.7).

To evaluate pmEV entrance into and/or presence in DCs, 250,000 DCs are seeded on a round cover slip in complete RPMI-1640 medium and are then incubated with pmEVs from single bacterial strains or combinations pmEVs at various ratios. Purified pmEVs may be labeled with fluorochromes or fluorescent proteins. After incubation for various timepoints (e.g., 1 hour, 2 hours), the cells are washed twice with ice-cold PBS and detached from the plate using trypsin. Cells are either allowed to remain intact or are lysed. Samples are then processed for flow cytometry. Total internalized pmEVs are quantified from lysed samples, and percentage of cells that uptake pmEVs is measured by counting fluorescent cells. The methods described above may also be performed in substantially the same manner using macrophages or epithelial cell lines (obtained from the ATCC) in place of DCs.

### Example 21: In Vitro Screening of pmEVs with an Enhanced Ability to Activate NK Cell Killing when Incubated with Target Cells

To demonstrate the ability of the selected pmEV compositions to elicit potent NK cell cytotoxicity to tumor cells, the following in vitro assay is used. Briefly, mononuclear cells from heparinized blood are obtained from healthy human donors. Optionally, an expansion step to increase the numbers of NK cells is performed as previously described (e.g., see Somanschi et al., J Vis Exp. 2011; (48):2540). The cells may be adjusted to a concentration of, cells/ml in RPMI-1640 medium containing 5% human serum. The PMNC cells are then labeled with appropriate antibodies and NK cells are isolated through FACS as CD3−/CD56+ cells and are ready for the subsequent cytotoxicity assay. Alternatively, NK cells are isolated using the autoMACs instrument and NK cell isolation kit following manufacturer's instructions (Miltenyl Biotec).

NK cells are counted and plated in a 96 well format with 20,000 or more cells per well, and incubated with single-strain pmEVs, with or without addition of antigen presenting cells (e.g., monocytes derived from the same donor), pmEVs from mixtures of bacterial strains, and appropriate controls. After 5-24 hours incubation of NK cells with pmEVs, pmEVs are removed from cells with PBS washes, NK cells are resuspended in 10 mL fresh media with antibiotics and are added to 96-well plates containing 20.000 target tumor cells/well. Mouse tumor cell lines used include B16.F10, SIY+B16.F10, and others. Human tumor cell lines are HLA-matched to donor, and can include PANC-1, UNKPC960/961, UNKC, and HELA cell lines. Plates are incubated for 2-24 hours at 37° C. under normal oxygen conditions. Staurospaurine is used as negative control to account for cell death.

Following this incubation, flow cytometry is used to measure tumor cell death using methods known in the art. Briefly, tumor cells are stained with viability dye. FACS analysis is used to gate specifically on tumor cells and measure the percentage of dead (killed) tumor cells. Data are also displayed as the absolute number of dead tumor cells per well.

This NK cell stimulation protocol may be repeated using combinations of purified pmEVs and live bacterial strains to maximize immune stimulation potential.

### Example 22: Using In Vitro Immune Activation Assays to Predict In Vivo Cancer Immunotherapy Efficacy of pmEV Compositions

In vitro immune activation assays identify pmEVs that are able to stimulate dendritic cells, which in turn activate CD8+ T cell killing. Therefore, the in vitro assays described above are used as a predictive screen of a large number of candidate pmEVs for potential immunotherapy activity. pmEVs that display enhanced stimulation of dendritic cells, enhanced stimulation of CD8+ T cell killing, enhanced stimulation of PBMC killing, and/or enhanced stimulation of NK cell killing, are preferentially chosen for in vivo cancer immunotherapy efficacy studies.

### Example 23: Determining the Biodistribution of pmEVs when Delivered Orally to Mice

Wild-type mice (e.g., C57BL/6 or BALB/c) are orally inoculated with the pmEV composition of interest to determine the in vivo biodistribution profile of purified pmEVs. pmEVs are labeled to aide in downstream analyses. Alternatively, tumor-bearing mice or mice with some immune disorder (e.g., systemic lupus erythematosus, experimental autoimmune encephalomyelitis, NASH) may be studied for in vivo distribution of pmEVs over a given time-course.

Mice can receive a single dose of the pmEV (e.g., 25-100 μg) or several doses over a defined time course (25-100 μg). Alternatively, pmEVs dosages may be administered based on particle count (e.g., 7e+08 to 6e+11 particles). Mice are housed under specific pathogen-free conditions following approved protocols. Alternatively, mice may be bred and maintained under sterile, germ-free conditions. Blood, stool, and other tissue samples can be taken at appropriate time points.

The mice are humanely sacrificed at various time points (i.e., hours to days) post administration of the pmEV compositions, and a full necropsy under sterile conditions is performed. Following standard protocols, lymph nodes, adrenal glands, liver, colon, small intestine, cecum, stomach, spleen, kidneys, bladder, pancreas, heart, skin, lungs, brain, and other tissue of interest are harvested and are used directly or snap frozen for further testing. The tissue samples are dissected and homogenized to prepare single-cell suspensions following standard protocols known to one skilled in the art. The number of pmEVs present in the sample is then quantified through flow cytometry. Quantification may also proceed with use of fluorescence microscopy after appropriate processing of whole mouse tissue (Vankelecom H., Fixation and paraffin-embedding of mouse tissues for GFP visualization, Cold Spring Harb. Protoc., 2009). Alternatively, the animals may be analyzed using live-imaging according to the pmEV labeling technique.

Biodistribution may be performed in mouse models of cancer such as but not limited to CT-26 and B16 (see, e.g., Kim et al., Nature Communications vol. 8, no. 626 (2017)) or autoimmunity such as but not limited to EAE and DTH (see, e.g., Turjeman et al., PLoS One 10(7): c0130442 (20105).

### Example 24: Purification and Preparation of Secreted Microbial Extracellular Vesicles (smEVs) from Bacteria

### Purification

Secreted microbial extracellular vesicles (smEVs) are purified and prepared from bacterial cultures (e.g., bacteria from Table 1 and/or Table 2) using methods known to those skilled in the art (S. Bin Park, et al. PLoS ONE. 6(3):e17629 (2011)).

For example, bacterial cultures are centrifuged at 10,000-15,500×g for 10-40 min at 4° C. or room temperature to pellet bacteria. Culture supernatants are then filtered to include material ≥0.22 μm (for example, via a 0.22 μm or 0.45 μm filter) and to exclude intact bacterial cells. Filtered supernatants are concentrated using methods that may include, but are not limited to, ammonium sulfate precipitation, ultracentrifugation, or filtration. Briefly, for ammonium sulfate precipitation, 1.5-3 M ammonium sulfate is added to filtered supernatant slowly, while stirring at 4° C. Precipitations are incubated at 4° C. for 8-48 hours and then centrifuged at 11,000×g for 20-40 min at 4° C. The pellets contain smEVs and other debris. Briefly, using ultracentrifugation, filtered supernatants are centrifuged at 100,000-200,000×g for 1-16 hours at 4° C. The pellet of this centrifugation contains smEVs and other debris. Briefly, using a filtration technique, using an Amicon Ultra spin filter or by tangential flow filtration, supernatants are filtered so as to retain species of molecular weight >50, 100, 300, or 500 kDa.

Alternatively, smEVs are obtained from bacterial cultures continuously during growth, or at selected time points during growth, by connecting a bioreactor to an alternating tangential flow (ATF) system (e.g., XCell ATF from Repligen) according to manufacturer's instructions. The ATF system retains intact cells (>0.22 μm) in the bioreactor, and allows smaller components (e.g., smEVs, free proteins) to pass through a filter for collection. For example, the system may be configured so that the <0.22 μm filtrate is then passed through a second filter of 100 kDa, allowing species such as smEVs between 0.22 μm and 100 kDa to be collected, and species smaller than 100 kDa to be pumped back into the bioreactor. Alternatively, the system may be configured to allow for medium in the bioreactor to be replenished and/or modified during growth of the culture. smEVs collected by this method may be further purified and/or concentrated by ultracentrifugation or filtration as described above for filtered supernatants.

smEVs obtained by methods described above may be further purified by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in 45% Optiprep in PBS. If filtration was used to concentrate the filtered supernatant, the concentrate is diluted using 60% Optiprep to a final concentration of 45% Optiprep. Samples are applied to a 0-45% discontinuous sucrose gradient and centrifuged at 200,000×g for 3-24 hours at 4° C. Alternatively, high resolution density gradient fractionation could be used to separate smEVs based on density.

### Preparation

To confirm sterility and isolation of the smEV preparations, smEVs are serially diluted onto agar medium used for routine culture of the bacteria being tested and incubated using routine conditions. Non-sterile preparations are passed through a 0.22 μm filter to exclude intact cells. To further increase purity, isolated smEVs may be DNase or proteinase K treated.

Alternatively, for preparation of smEVs used for in vivo injections, purified smEVs are processed as described previously (G. Norheim, et al. PLoS ONE. 10(9): e0134353 (2015)). Briefly, after sucrose gradient centrifugation, bands containing smEVs are resuspended to a final concentration of 50 μg/mL in a solution containing 3% sucrose or other solution suitable for in vivo injection known to one skilled in the art. This solution may also contain adjuvant, for example aluminum hydroxide at a concentration of 0-0.5% (w/v).

To make samples compatible with further testing (e.g., to remove sucrose prior to TEM imaging or in vitro assays), samples are buffer exchanged into PBS or 30 mM Tris, pH 8.0 using filtration (e.g., Amicon Ultra columns), dialysis, or ultracentrifugation (following 15-fold or greater dilution in PBS, 200,000×g, 1-3 hours, 4° C.) and resuspension in PBS.

For all of these studies, smEVs may be heated, irradiated, and/or lyophilized prior to administration (as described in Example 49).

### Example 25: Manipulating Bacteria Through Stress to Produce Various Amounts of smEVs and/or to Vary Content of smEVs

Stress, and in particular envelope stress, has been shown to increase production of smEVs by some bacterial strains (1. MacDonald. M. Kuehn. J Bacteriol 195(13): doi: 10/1128/JB.02267-12). In order to vary production of smEVs by bacteria, bacteria are stressed using various methods.

Bacteria may be subjected to single stressors or stressors in combination. The effects of different stressors on different bacteria is determined empirically by varying the stress condition and determining the IC50 value (the conditions required to inhibit cell growth by 50%). smEV purification, quantification, and characterization occurs. smEV production is quantified (1) in complex samples of bacteria and smEVs by nanoparticle tracking analysis (NTA) or transmission electron microscopy (TEM); or (2) following smEV purification by NTA, lipid quantification, or protein quantification. smEV content is assessed following purification by methods described above.

### Antibiotic Stress

Bacteria are cultivated under standard growth conditions with the addition of sublethal concentrations of antibiotics. This may include 0.1-1 μg/mL chloramphenicol, or 0.1-0.3 μg/mL gentamicin, or similar concentrations of other antibiotics (e.g., ampicillin, polymyxin B). Host antimicrobial products such as lysozyme, defensins, and Reg proteins may be used in place of antibiotics. Bacterially-produced antimicrobial peptides, including bacteriocins and microcins may also be used.

### Temperature Stress

Bacteria are cultivated under standard growth conditions, but at higher or lower temperatures than are typical for their growth. Alternatively, bacteria are grown under standard conditions, and then subjected to cold shock or heat shock by incubation for a short period of time at low or high temperatures respectively. For example, bacteria grown at 37° C. are incubated for 1 hour at 4° C.-18° C. for cold shock or 42° C.-5(0′C for heat shock.

### Starvation and Nutrient Limitation

To induce nutritional stress, bacteria are cultivated under conditions where one or more nutrients are limited. Bacteria may be subjected to nutritional stress throughout growth or shifted from a rich medium to a poor medium. Some examples of media components that are limited are carbon, nitrogen, iron, and sulfur. An example medium is M9 minimal medium (Sigma-Aldrich), which contains low glucose as the sole carbon source. Media components are also manipulated by the addition of chelators such as EDTA and deferoxamine.

### Saturation

Bacteria are grown to saturation and incubated past the saturation point for various periods of time. Alternatively, conditioned media is used to mimic saturating environments during exponential growth. Conditioned media is prepared by removing intact cells from saturated cultures by centrifugation and filtration, and conditioned media may be further treated to concentrate or remove specific components.

### Salt Stress

Bacteria are cultivated in or exposed for brief periods to medium containing NaCl, bile salts, or other salts.

### UV Stress

UV stress is achieved by cultivating bacteria under a UV lamp or by exposing bacteria to UV using an instrument such as a Stratalinker (Agilent). UV may be administered throughout the entire cultivation period, in short bursts, or for a single defined period following growth.

### Reactive Oxygen Stress

Bacteria are cultivated in the presence of sublethal concentrations of hydrogen peroxide (250-1,000 μM) to induce stress in the form of reactive oxygen species. Anaerobic bacteria are cultivated in or exposed to concentrations of oxygen that are toxic to them.

### Detergent Stress

Bacteria are cultivated in or exposed to detergent, such as sodium dodecyl sulfate (SDS) or deoxycholate, pH stress.

Bacteria are cultivated in or exposed for limited times to media of different pH.

### Example 26: Preparation of smEV-Free Bacteria

Bacterial samples containing minimal amounts of smEVs are prepared. smEV production is quantified (1) in complex samples of bacteria and extracellular components by NTA or TEM: or (2) following smEV purification from bacterial samples, by NTA, lipid quantification, or protein quantification.
a. Centrifugation and washing: Bacterial cultures are centrifuged at 11,000×g to separate intact cells from supernatant (including free proteins and vesicles). The pellet is washed with buffer, such as PBS, and stored in a stable way (e.g., mixed with glycerol, flash frozen, and stored at −80° C.).
b. ATF: Bacteria and smEVs are separated by connection of a bioreactor to an ATF system. smEV-free bacteria are retained within the bioreactor, and may be further separated from residual smEVs by centrifugation and washing, as described above.
c. Bacteria are grown under conditions that are found to limit production of smEVs. Conditions that may be varied.

### Example 27: A Colorectal Carcinoma Model

To study the efficacy of smEVs in a tumor model, one of many cancer cell lines may be used according to rodent tumor models known in the art.

For example, female 6-8 week old Balb/c mice are obtained from Taconic (Germantown, N.Y.) or other vendor. 100,000 CT-26 colorectal tumor cells (ATCC CRL-2638) are resuspended in sterile PBS and inoculated in the presence of 50% Matrigel. CT-26 tumor cells are subcutaneously injected into one hind flank of each mouse. When tumor volumes reach an average of 100 mm³ (approximately 10-12 days following tumor cell inoculation), animals are distributed into various treatment groups (e.g., Vehicle; smEVs, with or without anti-PD-1 antibody). Antibodies are administered intraperitoneally (i.p.) at 200 μg/mouse (100 μl final volume) every four days, starting on day 1, for a total of 3 times (Q4D×3), and smEVs are administered orally or intravenously and at varied doses and varied times. For example, smEVs (5 μg) are intravenously (i.v.) injected every third day, starting on day 1 for a total of 4 times (Q3D×4) and mice are assessed for tumor growth. Some mice may be intravenously injected with smEVs at 10, 15, or 20 μg smEVs/mouse. Other mice may receive 25, 50, or 100 mg of smEVs per mouse. Alternatively, some mice receive between 7.0e+09 to 3.0e+12 smEV particles per dose.

Alternatively, when tumor volumes reach an average of 100 mm³ (approximately 10-12 days following tumor cell inoculation), animals are distributed into the following groups: 1) Vehicle; 2) smEVs; and 3) anti-PD-1 antibody. Antibodies are administered intraperitoneally (i.p.) at 200 ug/mouse (100 ul final volume) every four days, starting on day 1, and smEVs are administered intraperitoneally (i.p.) daily, starting on day 1 until the conclusion of the study.

When tumor volumes reached an average of 100 mm³ (approximately 10-12 days following tumor cell inoculation), animals were distributed into the following groups: 1) Vehicle; 2) anti-PD-1 antibody; and 3) smEV (7.0 e+10 particle count). Antibodies were administered intraperitoneally (i.p.) at 200 μg/mouse (100 μl final volume) every four days, starting on day 1, and smEVs were intravenously (i.v.) injected daily, starting on day 1 until the conclusion of the study and tumors measured for growth. At day 11, the smEV group exhibited tumor growth inhibition that was significantly better than that seen in the anti-PD-1 group. Welch's test is performed for treatment vs vehicle. In a study looking at dose-response of smEVs, the highest dose of smEVs demonstrated the greatest efficacy, although in a study with smEVs, higher doses do not necessarily correspond to greater efficacy.

### Example 28: Administering smEV Compositions to Treat Mouse Tumor Models

As described herein a mouse model of cancer is generated by subcutaneously injecting a tumor cell line or patient-derived tumor sample and allowing it to engraft into healthy mice. The methods provided herein may be performed using one of several different tumor cell lines including, but not limited to: B16-F10 or B16-F10-SIY cells as an orthotopic model of melanoma. Panc02 cells as an orthotopic model of pancreatic cancer (Maletzki et al., 2008, Gut 57:483-491), LLC1 cells as an orthotopic model of lung cancer, and RM-1 as an orthotopic model of prostate cancer. As an example, but without limitation, methods for studying the efficacy of smEVs in the B16-F10 model are provided in depth herein.

A syngeneic mouse model of spontaneous melanoma with a very high metastatic frequency is used to test the ability of bacteria to reduce tumor growth and the spread of metastases. The smEVs chosen for this assay are compositions that may display enhanced activation of immune cell subsets and stimulate enhanced killing of tumor cells in vitro. The mouse melanoma cell line B16-F10 is obtained from ATCC. The cells are cultured in vitro as a monolayer in RPMI medium, supplemented with 10% heat-inactivated fetal bovine serum and 1% penicillin/streptomycin at 37° C. in an atmosphere of 5% CO2 in air. The exponentially growing tumor cells are harvested by trypsinization, washed three times with cold 1×PBS, and a suspension of 5E6 cells/mi is prepared for administration. Female C57BL/6 mice are used for this experiment. The mice are 6-8 weeks old and weigh approximately 16-20 g. For tumor development, each mouse is injected SC into the flank with 100 μl of the B16-F10 cell suspension. The mice are anesthetized by ketamine and xylazine prior to the cell transplantation. The animals used in the experiment may be started on an antibiotic treatment via instillation of a cocktail of kanamycin (0.4 mg/ml), gentamicin, (0.035 mg/ml), colistin (850 U/ml), metronidazole (0.215 mg/ml) and vancomycin (0.045 mg/ml) in the drinking water from day 2 to 5 and an intraperitoneal injection of clindamycin (10 mg/kg) on day 7 after tumor injection.

The size of the primary flank tumor is measured with a caliper every 2-3 days and the tumor volume is calculated using the following formula: tumor volume=the tumor width×tumor length×0.5. After the primary tumor reaches approximately 100 mm³, the animals are sorted into several groups based on their body weight. The mice are then randomly taken from each group and assigned to a treatment group. smEV compositions are prepared as previously described. The mice are orally innoculated by gavage with approximately 7.0e+09 to 3.0e+12 smEV particles. Alternatively, smEVs are administered intravenously. Mice receive smEVs daily, weekly, bi-weekly, monthly, bi-monthly, or on any other dosing schedule throughout the treatment period. Mice may be IV injected with smEVs in the tail vein, or directly injected into the tumor. Mice can be injected with smEVs, with or without live bacteria, and/or smEVs with or without inactivated/weakened or killed bacteria. Mice can be injected or orally gavaged weekly or once a month. Mice may receive combinations of purified smEVs and live bacteria to maximize tumor-killing potential. All mice are housed under specific pathogen-free conditions following approved protocols. Tumor size, mouse weight, and body temperature are monitored every 3-4 days and the mice are humanely sacrificed 6 weeks after the B16-F10 mouse melanoma cell injection or when the volume of the primary tumor reaches 1000 mm³. Blood draws are taken weekly and a full necropsy under sterile conditions is performed at the termination of the protocol.

Cancer cells can be easily visualized in the mouse B16-F10 melanoma model due to their melanin production. Following standard protocols, tissue samples from lymph nodes and organs from the neck and chest region are collected and the presence of micro- and macro-metastases is analyzed using the following classification rule. An organ is classified as positive for metastasis if at least two micro-metastatic and one macro-metastatic lesion per lymph node or organ are found. Micro-metastases are detected by staining the paraffin-embedded lymphoid tissue sections with hematoxylin-eosin following standard protocols known to one skilled in the art. The total number of metastases is correlated to the volume of the primary tumor and it is found that the tumor volume correlates significantly with tumor growth time and the number of macro- and micro-metastases in lymph nodes and visceral organs and also with the sum of all observed metastases. Twenty-five different metastatic sites are identified as previously described (Bobek V., et al., Syngeneic lymph-node-targeting model of green fluorescent protein-expressing Lewis lung carcinoma. Clin. Exp. Metastasis, 2004:21(8):705-8).

The tumor tissue samples are further analyzed for tumor infiltrating lymphocytes. The CD8+ cytotoxic T cells can be isolated by FACS and can then be further analyzed using customized p/MHC class I microarrays to reveal their antigen specificity (see e.g., Deviren G., et al., Detection of antigen-specific T cells on p/MHC microarrays, J. Mol. Recognit., 2007 January-February; 20(1):32-8). CD4+ T cells can be analyzed using customized p/MHC class II microarrays.

At various timepoints, mice are sacrificed and tumors, lymph nodes, or other tissues may be removed for ex vivo flow cytometric analysis using methods known in the art. For example, tumors are dissociated using a Miltenyi tumor dissociation enzyme cocktail according to the manufacturer's instructions. Tumor weights are recorded and tumors are chopped then placed in 15 ml tubes containing the enzyme cocktail and placed on ice. Samples are then placed on a gentle shaker at 37° C. for 45 minutes and quenched with up to 15 ml complete RPMI Each cell suspension is strained through a 70 μm filter into a 50 ml falcon tube and centrifuged at 1000 rpm for 10 minutes. Cells are resuspended in FACS buffer and washed to remove remaining debris. If necessary, samples are strained again through a second 70 μm filter into a new tube. Cells are stained for analysis by flow cytometry using techniques known in the art. Staining antibodies can include anti-CD11c (dendritic cells), anti-CD80, anti-CD86, anti-CD40, anti-MHCII, anti-CD8a, anti-CD4, and anti-CD103. Other markers that may be analyzed include pan-immune cell marker CD45, T cell markers (CD3, CD4, CD8, CD25, Foxp3, T-bet, Gata3, Rorγt, Granzyme B, CD69, PD-1, CTLA-4), and macrophage/mycloid markers (CD11b, MHCII, CD206, CD40, CSF1R, PD-L1, Gr-1). In addition to immunophenotyping, serum cytokines can be analyzed including, but not limited to, TNFa, IL-17, IL-13, IL-12p70, IL12p40, IL-10, IL-6, IL-5, IL-4, IL-2, IL-1b, IFNy, GM-CSF, G-CSF, M-CSF, MIG, IP10, MIP1b, RANTES, and MCP-1. Cytokine analysis may be carried out immune cells obtained from lymph nodes or other tissue, and/or on purified CD45+ tumor-infiltrated immune cells obtained ex vivo. Finally, immunohistochemistry is carried out on tumor sections to measure T cells, macrophages, dendritic cells, and checkpoint molecule protein expression.

The same experiment is also performed with a mouse model of multiple pulmonary melanoma metastases. The mouse melanoma cell line B16-BL6 is obtained from ATCC and the cells are cultured in vitro as described above. Female C57BL/6 mice are used for this experiment. The mice are 6-8 weeks old and weigh approximately 16-20 g. For tumor development, each mouse is injected into the tail vein with 100 μl of a 2E6 cells/mi suspension of Bl6-BL6 cells. The tumor cells that engraft upon IV injection end up in the lungs.

The mice are humanely killed after 9 days. The lungs are weighed and analyzed for the presence of pulmonary nodules on the lung surface. The extracted lungs am bleached with Fekete's solution, which does not bleach the tumor nodules because of the melanin in the B16 cells though a small fraction of the nodules is amelanotic (i.e. white). The number of tumor nodules is carefully counted to determine the tumor burden in the mice. Typically, 200-250 pulmonary nodules are found on the lungs of the control group mice (i.e. PBS gavage).

The percentage tumor burden is calculated for the various treatment groups. Percentage tumor burden is defined as the mean number of pulmonary nodules on the lung surfaces of mice that belong to a treatment group divided by the mean number of pulmonary nodules on the lung surfaces of the control group mice.

The tumor biopsies and blood samples are submitted for metabolic analysis via LCMS techniques or other methods known in the art. Differential levels of amino acids, sugars, lactate, among other metabolites, between test groups demonstrate the ability of the microbial composition to disrupt the tumor metabolic state.

### RNA Seq to Determine Mechanism of Action

Dendritic cells are purified from tumors. Peyers patches, and mesenteric lymph nodes. RNAseq analysis is carried out and analyzed according to standard techniques known to one skilled in the art (Z. Hou. Scientific Reports. 5(9570):doi:10.1038/srep09570 (2015)). In the analysis, specific attention is placed on innate inflammatory pathway genes including TLRs, CLRs, NLRs, and STING, cytokines, chemokines, antigen processing and presentation pathways, cross presentation, and T cell co-stimulation.

Rather than being sacrificed, some mice may be rechallenged with tumor cell injection into the contralateral flank (or other area) to determine the impact of the immune system's memory response on tumor growth.

### Example 29: Administering smEVs to Treat Mouse Tumor Models in Combination with PD-1 or PD-L1 Inhibition

To determine the efficacy of smEVs in tumor mouse models in combination with PD-1 or PD-L1 inhibition, a mouse tumor model may be used as described above.

smEVs are tested for their efficacy in the mouse tumor model, either alone or in combination with whole bacterial cells and with or without anti-PD-1 or anti-PD-L1. smEVs, bacterial cells, and/or anti-PD-1 or anti-PD-L1 are administered at varied time points and at varied doses. For example, on day 10 after tumor injection, or after the tumor volume reaches 100 mm³, the mice are treated with smEVs alone or in combination with anti-PD-1 or anti-PD-L1.

Mice may be administered smEVs orally, intravenously, or intratumorally. For example, some mice are intravenously injected with anywhere between 7.0e+09 to 3.0e+12 smEV particles. While some mice receive smEVs through i.v. injection, other mice may receive smEVs through intraperitoneal (i.p.) injection, subcutaneous (s.c.) injection, nasal route administration, oral gavage, or other means of administration. Some mice may receive smEVs every day (e.g., starting on day 1), while others may receive smEVs at alternative intervals (e.g., every other day, or once every three days). Groups of mice may be administered a pharmaceutical composition of the invention comprising a mixture of smEVs and bacterial cells. For example, the composition may comprise smEV particles and whole bacteria in a ratio from 1:1 (smEVs: bacterial cells) to 1-1×10¹²:1 (smEVs: bacterial cells).

Alternatively, some groups of mice may receive between 1×10⁴ and 5×10⁹ bacterial cells in an administration separate from, or comingled with, the smEV administration. As with the smEVs, bacterial cell administration may be varied by route of administration, dose, and schedule. The bacterial cells may be live, dead, or weakened. The bacterial cells may be harvested fresh (or frozen) and administered, or they may be irradiated or heat-killed prior to administration with the smEVs.

Some groups of mice are also injected with effective doses of checkpoint inhibitor For example, mice receive 100 μg anti-PD-L1 mAB (clone 10f.9g2, BioXCell) or another anti-PD-1 or anti-PD-L1 mAB in 100 μl PBS, and some mice receive vehicle and/or other appropriate control (e.g., control antibody). Mice are injected with mABs 3, 6, and 9 days after the initial injection. To assess whether checkpoint inhibition and smEV immunotherapy have an additive anti-tumor effect, control mice receiving anti-PD-1 or anti-PD-L1 mABs are included to the standard control panel. Primary (tumor size) and secondary (tumor infiltrating lymphocytes and cytokine analysis) endpoints are assessed, and some groups of mice may be rechallenged with a subsequent tumor cell inoculation to assess the effect of treatment on memory response.

### Example 30: Labeling Bacterial smEVs

smEVs may be labeled in order to track their biodistribution in vivo and to quantify and track cellular localization in various preparations and in assays conducted with mammalian cells. For example, smEVs may be radio-labeled, incubated with dyes, fluorescently labeled, luminescently labeled, or labeled with conjugates containing metals and isotopes of metals.

For example, smEVs may be incubated with dyes conjugated to functional groups such as NHS-ester, click-chemistry groups, streptavidin or biotin. The labeling reaction may occur at a variety of temperatures for minutes or hours, and with or without agitation or rotation. The reaction may then be stopped by adding a reagent such as bovine scrum albumin (BSA), or similar agent, depending on the protocol, and free or unbound dye molecule removed by ultra-centrifugation, filtration, centrifugal filtration, column affinity purification or dialysis. Additional washing steps involving wash buffers and vortexing or agitation may be employed to ensure complete removal of free dyes molecules such as described in Su Chul Jang et al, Small. 11. No. 4, 456-461(2017).

Fluorescently labeled smEVs are detected in cells or organs, or in in vitro and/or ex vivo samples by confocal microscopy, nanoparticle tracking analysis, flow cytometry, fluorescence activated cell sorting (FACs) or fluorescent imaging system such as the Odyssey CLx LICOR (see e.g., Wiklander et al. 2015. J. Extracellular Vesicles. 4:10 3402/jev.v4.26316). Additionally, fluorescently labeled smEVs are detected in whole animals and/or dissected organs and tissues using an instrument such as the IVIS spectrum CT (Perkin Elmer) or Pearl Imager, as in H-I. Choi, et al. Experimental & Molecular Medicine. 49: e330 (2017).

smEVs may be labeled with conjugates containing metals and isotopes of metals using the protocols described above. Metal-conjugated smEVs may be administered in vivo to animals. Cells may then be harvested from organs at various time-points, and analyzed ex vivo. Alternatively, cells derived from animals, humans, or immortalized cell lines may be treated with metal-labelled smEVs in vitro and cells subsequently labelled with metal-conjugated antibodies and phenotyped using a Cytometry by Time of Flight (CyTOF) instrument such as the Helios CyTOF (Fluidigm) or imaged and analyzed using and Imaging Mass Cytometry instrument such as the Hyperion Imaging System (Fluidigm). Additionally, smEVs may be labelled with a radioisotope to track the smEVs biodistribution (see, e.g., Miller et al., Nanoscale. 2014 May 7:6(9):4928-35).

### Example 31: Transmission Electron Microscopy to Visualize Purified Bacterial smEVs

smEVs are purified from bacteria batch cultures. Transmission electron microscopy (TEM) may be used to visualize purified bacterial smEVs (S. Bin Park, et al. PLoS ONE. 6(3):e17629 (2011). smEVs are mounted onto 300- or 400-mesh-size carbon-coated copper grids (Electron Microscopy Sciences, USA) for 2 minutes and washed with deionized water. smEVs are negatively stained using 2% (w/v) uranyl acetate for 20 sec-1 min. Copper grids are washed with sterile water and dried. Images are acquired using a transmission electron microscope with 100-120 kV acceleration voltage. Stained smEVs appear between 20-600 nm in diameter and are electron dense. 10-50 fields on each grid are screened.

### Example 32: Profiling smEV Composition and Content

smEVs may be characterized by any one of various methods including, but not limited to, NanoSight characterization, SDS-PAGE gel electrophoresis, Western blot, ELISA, liquid chromatography-mass spectrometry and mass spectrometry, dynamic light scattering, lipid levels, total protein, lipid to protein ratios, nucleic acid analysis and/or zeta potential.

NanoSight Characterization of smEVs

Nanoparticle tracking analysis (NTA) is used to characterize the size distribution of purified smEVs. Purified smEV preparations are run on a NanoSight machine (Malvern Instruments) to assess smEV size and concentration.

### SDS-PAGE Gel Electrophoresis

To identify the protein components of purified smEVs, samples are run on a gel, for example a Bolt Bis-Tris Plus 4-12% gel (Thermo-Fisher Scientific), using standard techniques. Samples are boiled in 1×SDS sample buffer for 10 minutes, cooled to 4° C. and then centrifuged at 16,000×g for 1 min. Samples are then run on a SDS-PAGE gel and stained using one of several standard techniques (e.g., Silver staining, Coomassie Blue, Gel Code Blue) for visualization of bands.

### Western Blot Analysis

To identify and quantify specific protein components of purified smEVs, smEV proteins are separated by SDS-PAGE as described above and subjected to Western blot analysis (Cvjetkovic et al., Sci. Rep. 6, 36338 (2016)) and are quantified via ELISA. smEV proteomics and Liquid Chromatography-Mass Spectrometry (LC-MS/MS) and Mass Spectrometry (MS)

Proteins present in smEVs are identified and quantified by Mass Spectrometry techniques. smEV proteins may be prepared for LC-MS/MS using standard techniques including protein reduction using dithiotreitol solution (DTT) and protein digestion using enzymes such as LysC and trypsin as described in Erickson et al, 2017 (Molecular Cell, VOLUME 65, ISSUE 2, P361-370, Jan. 19, 2017) Alternatively, peptides are prepared as described by Liu et al. 2010 (JOURNAL OF BACTERIOLOGY, June 2010, p. 2852-2860 Vol. 192, No. 11), Kieselbach and Oscarsson 2017 (Data Brief. 2017 February; 10: 426-431), Vildhede et al, 2018 (Drug Metabolism and Disposition Feb. 8, 2018). Following digestion, peptide preparations are run directly on liquid chromatography and mass spectrometry devices for protein identification within a single sample. For relative quantitation of proteins between samples, peptide digests from different samples are labeled with isobaric tags using the iTRAQ Reagent-8plex Multiplex Kit (Applied Biosystems, Foster City, Calif.) or TMT 10plex and 11plex Label Reagents (Thermo Fischer Scientific, San Jose, Calif., USA). Each peptide digest is labeled with a different isobaric tag and then the labeled digests are combined into one sample mixture. The combined peptide mixture is analyzed by LC-MS/MS for both identification and quantification. A database search is performed using the LC-MS/MS data to identify the labeled peptides and the corresponding proteins. In the case of isobaric labeling, the fragmentation of the attached tag generates a low molecular mass reporter ion that is used to obtain a relative quantitation of the peptides and proteins present in each smEV.

Additionally, metabolic content is ascertained using liquid chromatography techniques combined with mass spectrometry. A variety of techniques exist to determine metabolomic content of various samples and are known to one skilled in the art involving solvent extraction, chromatographic separation and a variety of ionization techniques coupled to mass determination (Roberts et al 2012 Targeted Metabolomics. Curr Protoc Mol Biol. 30: 1-24; Dettmer et al 2007, Mass spectrometry-based metabolomics. Mass Spectrom Rev. 26(1):51-78). As a non-limiting example, a LC-MS system includes a 4000 QTRAP triple quadrupole mass spectrometer (AB SCIEX) combined with 1100 Series pump (Agilent) and an HTS PAL autosampler (Leap Technologies). Media samples or other complex metabolic mixtures (˜10 μL) are extracted using nine volumes of 74.9:24.9:0.2 (v/v/v) acetonitrile/methanol/formic acid containing stable isotope-labeled internal standards (valine-d8, Isotec; and phenylalanine-d8, Cambridge Isotope Laboratories). Standards may be adjusted or modified depending on the metabolites of interest. The samples are centrifuged (10 minutes, 9,000×g, 4° C.), and the supernatants (10 μL) are submitted to LCMS by injecting the solution onto the HILIC column (150×2.1 mm, 3 μm particle size). The column is eluted by flowing a 5% mobile phase [10 mM ammonium formate, 0.1% formic acid in water] for 1 minute at a rate of 250 μl/minute followed by a linear gradient over 10 minutes to a solution of 40% mobile phase [acetonitrile with 0.1% formic acid]. The ion spray voltage is set to 4.5 kV and the source temperature is 450° C.

The data are analyzed using commercially available software like Multiquant 1.2 from AB SCIEX for mass spectrum peak integration. Peaks of interest should be manually curated and compared to standards to confirm the identity of the peak. Quantitation with appropriate standards is performed to determine the number of metabolites present in the initial media, after bacterial conditioning and after tumor cell growth. A non-targeted metabolomics approach may also be used using metabolite databases, such as but not limited to the NIST database, for peak identification.

### Dynamic Light Scattering (DLS)

DLS measurements, including the distribution of particles of different sizes in different smEV preparations are taken using instruments such as the DynaPro NanoStar (Wyatt Technology) and the Zetasizer Nano ZS (Malvern Instruments).

### Lipid Levels

Lipid levels are quantified using FM4-64 (Life Technologies), by methods similar to those described by A. J. McBroom et al. J Bacteriol 188:5385-5392, and A. Frias, et al. Microb Ecol. 59:476-486 (2010). Samples are incubated with FM4-64 (3.3 μg/mL in PBS for 10 minutes at 37° C. in the dark). After excitation at 515 nm, emission at 635 nm is measured using a Spectramax M5 plate reader (Molecular Devices). Absolute concentrations are determined by comparison of unknown samples to standards (such as palmitoyloleoylphosphatidylglycerol (POPG) vesicles) of known concentrations. Lipidomics can be used to identify the lipids present in the smEVs.

### Total Protein

Protein levels are quantified by standard assays such as the Bradford and BCA assays. The Bradford assays are run using Quick Start Bradford 1× Dye Reagent (Bio-Rad), according to manufacturer's protocols. BCA assays are run using the Pierce BCA Protein Assay Kit (Thermo-Fisher Scientific). Absolute concentrations are determined by comparison to a standard curve generated from BSA of known concentrations. Alternatively, protein concentration can be calculated using the Beer-Lambert equation using the sample absorbance at 280 nm (A280) as measured on a Nanodop spectrophotometer (Thermo-Fisher Scientific). In addition, proteomics may be used to identify proteins in the sample.

### Lipid:Protein Ratios

Lipid:protein ratios are generated by dividing lipid concentrations by protein concentrations. These provide a measure of the purity of vesicles as compared to free protein in each preparation.

### Nucleic Acid Analysis

Nucleic acids are extracted from smEVs and quantified using a Qubit fluorometer. Size distribution is assessed using a BioAnalyzer and the material is sequenced.

### Zeta Potential

The zeta potential of different preparations are measured using instruments such as the Zetasizer ZS (Malvern Instruments).

### Example 33: In Vitro Screening of smEVs for Enhanced Activation of Dendritic Cells

In vitro immune responses are thought to simulate mechanisms by which immune responses are induced in vivo, e.g., as in response to a cancer microenvironment. Briefly, PBMCs are isolated from heparinized venous blood from healthy donors by gradient centrifugation using Lymphoprep (Nycomed, Oslo, Norway), or from mouse spleens or bone marrow using the magnetic bead-based Human Blood Dendritic cell isolation kit (Miltenyi Biotech, Cambridge, Mass.). Using anti-human CD14 mAb, the monocytes are purified by Moflo and cultured in cRPMI at a cell density of 5e5 cells/ml in a 96-well plate (Costar Corp) for 7 days at 37° C. For maturation of dendritic cells, the culture is stimulated with 0.2 ng/mL IL-4 and 1000 U/ml GM-CSF at 37° C. for one week. Alternatively, maturation is achieved through incubation with recombinant GM-CSF for a week, or using other methods known in the art. Mouse DCs can be harvested directly from spleens using bead enrichment or differentiated from hematopoietic stem cells. Briefly, bone marrow may be obtained from the femurs of mice. Cells are recovered and red blood cells lysed. Stem cells are cultured in cell culture medium in 20 ng/ml mouse GMCSF for 4 days. Additional medium containing 20 ng/ml mouse GM-CSF is added. On day 6 the medium and non-adherent cells are removed and replaced with fresh cell culture medium containing 20 ng/ml GMCSF. A final addition of cell culture medium with 20 ng/ml GM-CSF is added on day 7. On day 10, non-adherent cells are harvested and seeded into cell culture plates overnight and stimulated as required Dendritic cells are then treated with various doses of smEVs with or without antibiotics. For example, 25-75 μg/mL smEVs for 24 hours with antibiotics. smEV compositions tested may include smEVs from a single bacterial species or strain, or a mixture of smEVs from one or more genus, 1 or more species, or 1 or more strains (e.g., one or more strains within one species). PBS is included as a negative control and LPS, anti-CD40 antibodies, and/or smEVs are used as positive controls. Following incubation, DCs are stained with anti CD11b, CD11c, CD103, CD8a, CD40, CD80, CD83, CD86, MHCI and MHCII, and analyzed by flow cytometry. DCs that are significantly increased in CD40, CD80, CD83, and CD86 as compared to negative controls are considered to be activated by the associated bacterial smEV composition. These experiments are repeated three times at minimum.

To screen for the ability of smEV-activated epithelial cells to stimulate DCs, the above protocol is followed with the addition of a 24-hour epithelial cell smEV co-culture prior to incubation with DCs. Epithelial cells are washed after incubation with smEVs and are then co-cultured with DCs in an absence of smEVs for 24 hours before being processed as above. Epithelial cell lines may include Int407, HEL293, HT29, T84 and CACO2.

As an additional measure of DC activation, 100 μl of culture supernatant is removed from wells following 24-hour incubation of DCs with smEVs or smEV-treated epithelial cells and is analyzed for secreted cytokines, chemokines, and growth factors using the multiplexed Luminex Magpix. Kit (EMD Millipore, Darmstadt, Germany). Briefly, the wells are pre-wet with buffer, and 25 μl of 1× antibody-coated magnetic beads are added and 2×200 μl of wash buffer are performed in every well using the magnet. 50 μl of Incubation buffer, 50 μl of diluent and 50 μl of samples are added and mixed via shaking for 2 hrs at room temperature in the dark. The beads are then washed twice with 200 μl wash buffer. 100 μl of 1× biotinylated detector antibody is added and the suspension is incubated for 1 hour with shaking in the dark. Two, 200 μl washes are then performed with wash buffer. 100 μl of 1×SAV-RPE reagent is added to each well and is incubated for 30 min at RT in the dark. Three 200 μl washes are performed and 125 μl of wash buffer is added with 2-3 min shaking occurs. The wells are then submitted for analysis in the Luminex xMAP system.

Standards allow for careful quantitation of the cytokines including GM-CSF, IFN-g, IFN-a, IFN-B, IL-1a, IL-1B, IL-2, IL-4, IL-5, IL-6, IL-8, IL-10, IL-13, IL-12 (p40/p70), IL-17A, IL-17F, IL-21, IL-22 TL-23, IL-25, IP-10, KC, MCP-1, MIG, MIP1a, TNFa, and VEGF. These cytokines are assessed in samples of both mouse and human origin. Increases in these cytokines in the bacterial treated samples indicate enhanced production of proteins and cytokines from the host. Other variations on this assay examining specific cell types ability to release cytokines are assessed by acquiring these cells through sorting methods and are recognized by one of ordinary skill in the art. Furthermore, cytokine mRNA is also assessed to address cytokine release in response to an smEV composition.

This DC stimulation protocol may be repeated using combinations of purified smEVs and live bacterial strains to maximize immune stimulation potential.

### Example 34: In Vitro Screening of smEVs for Enhanced Activation of CD8+ T Cell Killing when Incubated with Tumor Cells

In vitro methods for screening smEVs that can activate CD8+ T cell killing of tumor cells are described. Briefly. DCs are isolated from human PBMCs or mouse spleens, using techniques known in the art, and incubated in vitro with single-strain smEVs, mixtures of smEVs, and/or appropriate controls. In addition, CD8+ T cells are obtained from human PBMCs or mouse spleens using techniques known in the art, for example the magnetic bead-based Mouse CD8a+ T Cell Isolation Kit and the magnetic bead-based Human CD8+ T Cell Isolation Kit (both from Miltenyi Biotech, Cambridge. MA). After incubation of DCs with smEVs for some time (e.g., for 24-hours), or incubation of DCs with smEV-stimulated epithelial cells, smEVs are removed from the cell culture with PBS washes and 100 ul of fresh media with antibiotics is added to each well, and 200,000 T cells are added to each experimental well in the 96-well plate. Anti-CD3 antibody is added at a final concentration of 2 ug/ml. Co-cultures are then allowed to incubate at 37° C. for 96 hours under normal oxygen conditions.

For example, approximately 72 hours into the coculture incubation, tumor cells are plated for use in the assay using techniques known in the art. For example, 50,000 tumor cells/well are plated per well in new 96-well plates. Mouse tumor cell lines used may include B16.F10, SIY+B16.F10, and others. Human tumor cell lines are HLA-matched to donor, and can include PANC-1, UNKPC960/961, UNKC, and HELA cell lines. After completion of the 96-hour co-culture, 100 μl of the CD8+ T cell and DC mixture is transferred to wells containing tumor cells. Plates are incubated for 24 hours at 37° C. under normal oxygen conditions. Staurospaurine may be used as negative control to account for cell death.

Following this incubation, flow cytometry is used to measure tumor cell death and characterize immune cell phenotype. Briefly, tumor cells are stained with viability dye. FACS analysis is used to gate specifically on tumor cells and measure the percentage of dead (killed) tumor cells. Data are also displayed as the absolute number of dead tumor cells per well. Cytotoxic CD8+ T cell phenotype may be characterized by the following methods: a) concentration of supernatant granzyme B, IFNy and TNFa in the culture supernatant as described below, b) CD8+ T cell surface expression of activation markers such as DC69. CD25, CD154, PD-1, gamma/delta TCR, Foxp3, T-bet, granzyme B, c) intracellular cytokine staining of IFNy, granzyme B, TNFa in CD8+ T cells. CD4+ T cell phenotype may also be assessed by intracellular cytokine staining in addition to supernatant cytokine concentration including INFy, TNFa, IL-12, IL-4, IL-5, IL-17, IL-10, chemokines etc.

As an additional measure of CD8+ T cell activation, 100 μl of culture supernatant is removed from wells following the 96-hour incubation of T cells with DCs and is analyzed for secreted cytokines, chemokines, and growth factors using the multiplexed Luminex Magpix. Kit (EMD Millipore, Darmstadt, Germany). Briefly, the wells are pre-wet with buffer, and 25 μl of 1× antibody-coated magnetic beads are added and 2×200 μl of wash buffer are performed in every well using the magnet. 50 μl of Incubation buffer, 50 μl of diluent and 50 μl of samples are added and mixed via shaking for 2 hrs at room temperature in the dark. The beads are then washed twice with 200 μl wash buffer. 100 μl of 1× biotinylated detector antibody is added and the suspension is incubated for 1 hour with shaking in the dark. Two, 200 μl washes are then performed with wash buffer. 100 μl of 1×SAV-RPE reagent is added to each well and is incubated for 30 min at RT in the dark. Three 200 μl washes are performed and 125 μl of wash buffer is added with 2-3 min shaking occurs. The wells are then submitted for analysis in the Luminex xMAP system.

Standards allow for careful quantitation of the cytokines including GM-CSF, IFN-g, IFN-a, IFN-B IL-1a, IL-1B, IL-2, IL-4, IL-5, IL-6, IL-8, IL-10, IL-13. IL-12 (p40/p70), IL-17, IL-23, IP-10, KC, MCP-1, MIG, MIP1a, TNFa, and VEGF. These cytokines are assessed in samples of both mouse and human origin. Increases in these cytokines in the bacterial treated samples indicate enhanced production of proteins and cytokines from the host. Other variations on this assay examining specific cell types ability to release cytokines are assessed by acquiring these cells through sorting methods and are recognized by one of ordinary skill in the art. Furthermore, cytokine mRNA is also assessed to address cytokine release in response to an smEV composition. These changes in the cells of the host stimulate an immune response similarly to in vivo response in a cancer microenvironment.

This CD8+ T cell stimulation protocol may be repeated using combinations of purified smEVs and live bacterial strains to maximize immune stimulation potential.

### Example 35: In Vitro Screening of smEVs for Enhanced Tumor Cell Killing by PBMCs

Various methods may be used to screen smEVs for the ability to stimulate PBMCs, which in turn activate CD8+ T cells to kill tumor cells. For example, PBMCs are isolated from heparinized venous blood from healthy human donors by ficoll-paque gradient centrifugation for mouse or human blood, or with Lympholyte Cell Separation Media (Cedarlane Labs, Ontario, Canada) from mouse blood PBMCs are incubated with single-strain smEVs, mixtures of smEVs, and appropriate controls. In addition, CD8+ T cells are obtained from human PBMCs or mouse spleens. After the 24-hour incubation of PBMCs with smEVs, smEVs are removed from the cells using PBS washes. 100 ul of fresh media with antibiotics is added to each well. An appropriate number of T cells (e.g., 200,000 T cells) are added to each experimental well in the 96-well plate. Anti-CD3 antibody is added at a final concentration of 2 μg/ml. Co-cultures are then allowed to incubate at 37° C. for 96 hours under normal oxygen conditions.

For example, 72 hours into the coculture incubation, 50,000 tumor cells/well are plated per well in new 96-well plates. Mouse tumor cell lines used include B16.F10, SIY+B16.F10, and others. Human tumor cell lines are HLA-matched to donor, and can include PANC-1, UNKPC960/961, UNKC, and HELA cell lines. After completion of the 96-hour co-culture, 100 μl of the CD8+ T cell and PBMC mixture is transferred to wells containing tumor cells. Plates are incubated for 24 hours at 37° C. under normal oxygen conditions. Staurospaurine is used as negative control to account for cell death.

Following this incubation, flow cytometry is used to measure tumor cell death and characterize immune cell phenotype. Briefly, tumor cells are stained with viability dye. FACS analysis is used to gate specifically on tumor cells and measure the percentage of dead (killed) tumor cells. Data are also displayed as the absolute number of dead tumor cells per well. Cytotoxic CD8+ T cell phenotype may be characterized by the following methods: a) concentration of supernatant granzyme B, IFNy and TNFa in the culture supernatant as described below, b) CD8+ T cell surface expression of activation markers such as DC69, CD25, CD154, PD-1, gamma/delta TCR. Foxp3, T-bet, granzyme B, c) intracellular cytokine staining of IFNy, granzyme B, TNFa in CD8+ T cells. CD4+ T cell phenotype may also be assessed by intracellular cytokine staining in addition to supernatant cytokine concentration including INFy, TNFa, IL-12, IL-4, IL-5, IL-17, IL-10, chemokines etc.

As an additional measure of CD8+ T cell activation, 100 μl of culture supernatant is removed from wells following the 96-hour incubation of T cells with DCs and is analyzed for secreted cytokines, chemokines, and growth factors using the multiplexed Luminex Magpix. Kit (EMD Millipore, Darmstadt, Germany). Briefly, the wells are pre-wet with buffer, and 25 μl of 1× antibody-coated magnetic beads are added and 2×200 μl of wash buffer are performed in every well using the magnet. 50 μl of Incubation buffer, 50 μl of diluent and 50 μl of samples are added and mixed via shaking for 2 hrs at room temperature in the dark. The beads are then washed twice with 200 μl wash buffer. 100 μl of 1× biotinylated detector antibody is added and the suspension is incubated for 1 hour with shaking in the dark. Two, 200 μl washes are then performed with wash buffer. 100 μl of 1×SAV-RPE reagent is added to each well and is incubated for 30 min at RT in the dark. Three 200 μl washes are performed and 125 μl of wash buffer is added with 2-3 min shaking occurs. The wells are then submitted for analysis in the Luminex xMAP system.

Standards allow for careful quantitation of the cytokines including GM-CSF, IFN-g, IFN-a, IFN-B IL-1a, IL-IB, IL-2, IL-4, IL-5, IL-6, IL-8, IL-10, IL-13, IL-12 (p40/p70), IL-17, IL-23, IP-10, KC, MCP-1, MIG, MIP1a, TNFa, and VEGF. These cytokines are assessed in samples of both mouse and human origin. Increases in these cytokines in the bacterial treated samples indicate enhanced production of proteins and cytokines from the host. Other variations on this assay examining specific cell types ability to release cytokines are assessed by acquiring these cells through sorting methods and are recognized by one of ordinary skill in the art. Furthermore, cytokine mRNA is also assessed to address cytokine release in response to an smEV composition. These changes in the cells of the host stimulate an immune response similarly to in vivo response in a cancer microenvironment.

This PBMC stimulation protocol may be repeated using combinations of purified smEVs with or without combinations of live, dead, or inactivated/weakened bacterial strains to maximize immune stimulation potential.

### Example 36: In Vitro Detection of smEVs in Antigen-Presenting Cells

Dendritic cells in the lamina propria constantly sample live bacteria, dead bacteria, and microbial products in the gut lumen by extending their dendrites across the gut epithelium, which is one way that smEVs produced by bacteria in the intestinal lumen may directly stimulate dendritic cells. The following methods represent a way to assess the differential uptake of smEVs by antigen-presenting cells. Optionally, these methods may be applied to assess immunomodulatory behavior of smEVs administered to a patient.

Dendritic cells (DCs) are isolated from human or mouse bone marrow, blood, or spleens according to standard methods or kit protocols (e.g., Inaba K. Swiggard W J, Steinman R M, Romani N. Schuler G, 2001. Isolation of dendritic cells. Current Protocols in Immunology. Chapter 3:Unit3.7).

To evaluate smEV entrance into and/or presence in DCs, 250,000 DCs are seeded on a round cover slip in complete RPMI-1640 medium and are then incubated with smEVs from single bacterial strains or combinations smEVs at various ratios. Purified smEVs may be labeled with fluorochromes or fluorescent proteins. After incubation for various timepoints (e.g., 1 hour, 2 hours), the cells are washed twice with ice-cold PBS and detached from the plate using trypsin. Cells are either allowed to remain intact or are lysed. Samples are then processed for flow cytometry Total internalized smEVs are quantified from lysed samples, and percentage of cells that uptake smEVs is measured by counting fluorescent cells. The methods described above may also be performed in substantially the same manner using macrophages or epithelial cell lines (obtained from the ATCC) in place of DCs.

### Example 37: In Vitro Screening of smEVs with an Enhanced Ability to Activate NK Cell Killing when Incubated with Target Cells

To demonstrate the ability of the selected smEV compositions to elicit potent NK cell cytotoxicity to tumor cells, the following in vitro assay is used. Briefly, mononuclear cells from heparinized blood are obtained from healthy human donors. Optionally, an expansion step to increase the numbers of NK cells is performed as previously described (e.g., see Somanschi et al., J Vis Exp. 2011; (48):2540). The cells may be adjusted to a concentration of cells/ml in RPMI-1640 medium containing 5% human serum. The PMNC cells are then labeled with appropriate antibodies and NK cells are isolated through FACS as CD3−/CD56+ cells and are ready for the subsequent cytotoxicity assay. Alternatively, NK cells are isolated using the autoMACs instrument and NK cell isolation kit following manufacturer's instructions (Miltenyl Biotec).

NK cells are counted and plated in a 96 well format with 20,000 or more cells per well, and incubated with single-strain smEVs, with or without addition of antigen presenting cells (e.g., monocytes derived from the same donor), smEVs from mixtures of bacterial strains, and appropriate controls. After 5-24 hours incubation of NK cells with smEVs, smEVs are removed from cells with PBS washes, NK cells are resuspended in 10 mL fresh media with antibiotics and are added to 96-well plates containing 20,000 target tumor cells/well. Mouse tumor cell lines used include B16.F10, SIY+B16.F10, and others. Human tumor cell lines are HLA-matched to donor, and can include PANC-1, UNKPC960/961, UNKC, and HELA cell lines. Plates are incubated for 2-24 hours at 37° C. under normal oxygen conditions. Staurospaurine is used as negative control to account for cell death.

Following this incubation, flow cytometry is used to measure tumor cell death using methods known in the art. Briefly, tumor cells are stained with viability dye. FACS analysis is used to gate specifically on tumor cells and measure the percentage of dead (killed) tumor cells. Data are also displayed as the absolute number of dead tumor cells per well.

This NK cell stimulation protocol may be repeated using combinations of purified smEVs and live bacterial strains to maximize immune stimulation potential.

### Example 38: Using In Vitro Immune Activation Assays to Predict In Vivo Cancer Immunotherapy Efficacy of smEV Compositions

In vitro immune activation assays identify smEVs that are able to stimulate dendritic cells, which in turn activate CD8+ T cell killing. Therefore, the in vitro assays described above are used as a predictive screen of a large number of candidate smEVs for potential immunotherapy activity. smEVs that display enhanced stimulation of dendritic cells, enhanced stimulation of CD8+ T cell killing, enhanced stimulation of PBMC killing, and/or enhanced stimulation of NK cell killing, are preferentially chosen for in vivo cancer immunotherapy efficacy studies.

### Example 39: Determining the Biodistribution of smEVs when Delivered Orally to Mice

Wild-type mice (e.g., C57BL/6 or BALB/c) are orally inoculated with the smEV composition of interest to determine the in vivo biodistribution profile of purified smEVs. smEVs are labeled to aide in downstream analyses. Alternatively, tumor-bearing mice or mice with some immune disorder (e.g., systemic lupus erythematosus, experimental autoimmune encephalomyelitis, NASH) may be studied for in vivo distribution of smEVs over a given time-course.

Mice can receive a single dose of the smEV (e.g., 25-100 μg) or several doses over a defined time course (25-100 μg). Alternatively, smEVs dosages may be administered based on particle count (e.g., 7e+08 to 6e+11 particles). Mice are housed under specific pathogen-free conditions following approved protocols. Alternatively, mice may be bred and maintained under sterile, germ-free conditions. Blood, stool, and other tissue samples can be taken at appropriate time points.

The mice are humanely sacrificed at various time points (i.e., hours to days) post administration of the smEV compositions, and a full necropsy under sterile conditions is performed. Following standard protocols, lymph nodes, adrenal glands, liver, colon, small intestine, cecum, stomach, spleen, kidneys, bladder, pancreas, heart, skin, lungs, brain, and other tissue of interest are harvested and are used directly or snap frozen for further testing. The tissue samples are dissected and homogenized to prepare single-cell suspensions following standard protocols known to one skilled in the art. The number of smEVs present in the sample is then quantified through flow cytometry. Quantification may also proceed with use of fluorescence microscopy after appropriate processing of whole mouse tissue (Vankelecom H., Fixation and paraffin-embedding of mouse tissues for GFP visualization, Cold Spring Harb. Protoc. 2009). Alternatively, the animals may be analyzed using live-imaging according to the smEV labeling technique.

Biodistribution may be performed in mouse models of cancer such as but not limited to CT-26 and B16 (see, e.g., Kim et al., Nature Communications vol 8, no. 626 (2017)) or autoimmunity such as but not limited to EAE and DTH (see, e.g., Turjeman et al., PLoS One 10(7): e0130442 (20105).

### Example 40: Manufacturing Conditions

Enriched media is used to grow and prepare the bacteria for in vitro and in vivo use and, ultimately, for pmEV and smEV preparations. For example, media may contain sugar, yeast extracts, plant-based peptones, buffers, salts, trace elements, surfactants, anti-foaming agents, and vitamins. Composition of complex components such as yeast extracts and peptones may be undefined or partially defined (including approximate concentrations of amino acids, sugars etc.). Microbial metabolism may be dependent on the availability of resources such as carbon and nitrogen. Various sugars or other carbon sources may be tested. Alternatively, media may be prepared and the selected bacterium grown as shown by Saarela et al. J. Applied Microbiology. 2005, 99: 1330-1339, which is hereby incorporated by reference. Influence of fermentation time, cryoprotectant and neutralization of cell concentrate on freeze-drying survival, storage stability, and acid and bile exposure of the selected bacterium produced without milk-based ingredients.

At large scale, the media is sterilized. Sterilization may be accomplished by Ultra High Temperature (UHT) processing. The UHT processing is performed at very high temperature for short periods of time. The UHT range may be from 135-180° C. For example, the medium may be sterilized from between 10 to 30 seconds at 135° C.

Inoculum can be prepared in flasks or in smaller bioreactors and growth is monitored. For example, the inoculum size may be between approximately 0.5 and 3% of the total bioreactor volume. Depending on the application and need for material, bioreactor volume can be at least 2 L, 10 L, 80 L, 100 L, 250 L, 1000 L, 2500 L, 5000 L, 10,000 L.

Before the inoculation, the bioreactor is prepared with medium at desired pH, temperature, and oxygen concentration. The initial pH of the culture medium may be different that the process set-point. pH stress may be detrimental at low cell centration; the initial pH could be between pH 7.5 and the process set-point. For example, pH may be set between 4.5 and 8.0. During the fermentation, the pH can be controlled through the use of sodium hydroxide, potassium hydroxide, or ammonium hydroxide. The temperature may be controlled from 25° C. to 45° C., for example at 37° C. Anaerobic conditions are created by reducing the level of oxygen in the culture broth from around 8 mg/L to Omg/L. For example, nitrogen or gas mixtures (N₂, CO₂, and Hz) may be used in order to establish anaerobic conditions. Alternatively, no gases are used and anaerobic conditions are established by cells consuming remaining oxygen from the medium. Depending on strain and inoculum size, the bioreactor fermentation time can vary. For example, fermentation time can vary from approximately 5 hours to 48 hours.

Reviving microbes from a frozen state may require special considerations. Production medium may stress cells after a thaw; a specific thaw medium may be required to consistently start a seed train from thawed material. The kinetics of transfer or passage of seed material to fresh medium, for the purposes of increasing the seed volume or maintaining the microbial growth state, may be influenced by the current state of the microbes (ex. exponential growth, stationary growth, unstressed, stressed).

Inoculation of the production fermenter(s) can impact growth kinetics and cellular activity. The initial state of the bioreactor system must be optimized to facilitate successful and consistent production. The fraction of seed culture to total medium (e.g., a percentage) has a dramatic impact on growth kinetics. The range may be 1-5% of the fermenter's working volume. The initial pH of the culture medium may be different from the process set-point. pH stress may be detrimental at low cell concentration; the initial pH may be between pH 7.5 and the process set-point. Agitation and gas flow into the system during inoculation may be different from the process set-points Physical and chemical stresses due to both conditions may be detrimental at low cell concentration.

Process conditions and control settings may influence the kinetics of microbial growth and cellular activity. Shifts in process conditions may change membrane composition, production of metabolites, growth rate, cellular stress, etc. Optimal temperature range for growth may vary with strain. The range may be 20-40° C. Optimal pH for cell growth and performance of downstream activity may vary with strain. The range may be pH 5-8. Gasses dissolved in the medium may be used by cells for metabolism. Adjusting concentrations of O₂, CO₂, and N₂ throughout the process may be required. Availability of nutrients may shift cellular growth. Microbes may have alternate kinetics when excess nutrients are available.

The state of microbes at the end of a fermentation and during harvesting may impact cell survival and activity. Microbes may be preconditioned shortly before harvest to better prepare them for the physical and chemical stresses involved in separation and downstream processing. A change in temperature (often reducing to 20-5° C.) may reduce cellular metabolism, slowing growth (and/or death) and physiological change when removed from the fermenter. Effectiveness of centrifugal concentration may be influenced by culture pH. Raising pH by 1-2 points can improve effectiveness of concentration but can also be detrimental to cells. Microbes may be stressed shortly before harvest by increasing the concentration of salts and/or sugars in the medium. Cells stressed m this way may better survive freezing and lyophilization during downstream.

Separation methods and technology may impact how efficiently microbes are separated from the culture medium. Solids may be removed using centrifugation techniques. Effectiveness of centrifugal concentration can be influenced by culture pH or by the use of flocculating agents. Raising pH by 1-2 points may improve effectiveness of concentration but can also be detrimental to cells. Microbes may be stressed shortly before harvest by increasing the concentration of salts and/or sugars in the medium. Cells stressed in this way may better survive freezing and lyophilization during downstream. Additionally, Microbes may also be separated via filtration. Filtration is superior to centrifugation techniques for purification if the cells require excessive g-minutes to successfully centrifuge. Excipients can be added before after separation. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets mixed with excipients are submerged in liquid nitrogen.

Harvesting can be performed by continuous centrifugation Product may be resuspended with various excipients to a desired final concentration. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets mixed with excipients are submerged in liquid nitrogen.

Lyophilization of material, including live bacteria, vesicles, or other bacterial derivative includes a freezing, primary drying, and secondary drying phase. Lyophilization begins with freezing. The product material may or may not be mixed with a lyoprotectant or stabilizer prior to the freezing stage. A product may be frozen prior to the loading of the lyophilizer, or under controlled conditions on the shelf of the lyophilizer. During the next phase, the primary drying phase, ice is removed via sublimation. Here, a vacuum is generated and an appropriate amount of heat is supplied to the material. The ice will sublime while keeping the product temperature below freezing, and below the material's critical temperature (T_{c}). The temperature of the shelf on which the material is loaded and the chamber vacuum can be manipulated to achieve the desired product temperature. During the secondary drying phase, product-bound water molecules are removed. Here, the temperature is generally raised higher than in the primary drying phase to break any physico-chemical interactions that have formed between the water molecules and the product material. After the freeze-drying process is complete, the chamber may be filled with an inert gas, such as nitrogen. The product may be sealed within the freeze dryer under dry conditions, in a glass vial or other similar container, preventing exposure to atmospheric water and contaminates.

### Example 41: smEV and pmEV Preparation

The smEVs and pmEVs may be prepared as follows.

smEVs: Downstream processing of smEVs begins immediately following harvest of the bioreactor. Centrifugation at 20,000×g is used to remove the cells from the broth. The resulting supernatant is clarified using 0.22 μm filter. The smEVs are concentrated and washed using tangential flow filtration (TFF) with flat sheet cassettes ultrafiltration (UF) membranes with 100 kDa molecular weight cutoff (MWCO). Diafiltration (DF) is used to washout small molecules and small proteins using 5 volumes of phosphate buffer solution (PBS). The retentate from TFF is spun down in an ultracentrifuge at 200,000×g for 1 hour to form a pellet rich in smEVs called a high-speed pellet (HSP). The pellet is resuspended with minimal PBS and a gradient is prepared with Optiprep™ density gradient medium and ultracentrifuged at 200,000×g for 16 hours. Of the resulting fractions, 2 middle bands contain smEVs. The fractions are washed with 15 fold PBS and the smEVs spun down at 200.000×g for 1 hr to create the fractionated HSP or fHSP. It is subsequently resuspended with minimal PBS, pooled, and analyzed for particles per mL and protein content. Dosing is prepared from the particle/mL count to achieve desired concentration. The smEVs are characterized using a NanoSight NS300 by Malvern Panalytical in scatter mode using the 532 nm laser.

pmEVs:

Cell pellets are removed from freezer and placed on ice. Pellet weights are noted.

Cold 100 mM Tris-HCl pH 7.5 is added to the frozen pellets and the pellets are thawed rotating at 4° C.

10 mg/ml DNase stock is added to the thawed pellets to a final concentration of 1 mg/mL.

The pellets are incubated on the inverter for 40 min at RT (room temperature).

The sample is filtered in a 70 um cell strainer before running through the Emulsiflex.

The samples are lysed using the Emulsiflex with 8 discrete cycles at 22.00 psi.

To remove the cellular debris from the lysed sample, the sample is centrifuged at 12,500×g, 15 min, 4° C.

The sample is centrifuged two additional times at 12,500×g, 15 min, 4° C. each time moving the supernatant to a fresh tube.

To pellet the membrane proteins, the sample is centrifuged at 120,000×g, 1 hr, 4° C.

The pellet is resuspended in 10 mL ice-cold 0.1 M sodium carbonate pH 11. The sample is incubated on the inverter at 4° C. for 1 hour.

The sample is centrifuged at 120,000×g, 1 hr, 4° C.

10 mL 100 mM Tris-HCl pH 7.5 is added to pellet and incubate O/N (overnight) at 4° C.

The pellet is resuspended and the sample is centrifuged at 120,000×g for 1 hour at 4° C.

The supernatant is discarded and the pellet is resuspended in a minimal volume of PBS.

Dosing pmEVs is based on particle counts, as assessed by Nanoparticle Tracking Analysis (NTA) using a NanoSight NS300 (Malvern Panalytical) according to manufacturer instructions. Counts for each sample are based on at least three videos of 30 sec duration each, counting 40-140 particles per frame.

Gamma irradiation: For gamma irradiation, pmEVs are prepared in frozen form and gamma irradiated on dry ice at 25 kGy radiation dose; whole microbe lyophilized powder is gamma irradiated at ambient temperature at 17.5 kGy radiation dose.

Lyophilization: Samples are placed in lyophilization equipment and frozen at −45° C. The lyophilization cycle include a hold step at −45° C. for 10 min. The vacuum begins and is set to 100 mTorr and the sample is held at −45° C. for another 10 min. Primary drying begins with a temperature ramp to −25° C. over 300 minutes and it is held at this temperature for 4630 min. Secondary drying start with a temperature ramp to 20° C. over 200 min while the vacuum is decreased to 20 mTorr. It is held at this temperature and pressure for 1200 min. The final step increases the temperature from 20 to 25° C. where it remains at a vacuum of 20 mTorr for 10 min.

### Example 42: Acutalibacter w. Strain A. Anaerotruncus colihominis Strain A. And Subdoligranulum variabile Strain A smEV: U937 Testing

smEVs from Acutalibacter sp. Strain A, Anaerotruncus colihominis Strain A, and Subdoligranulum variabile Strain A were tested in the U937 assay described above.

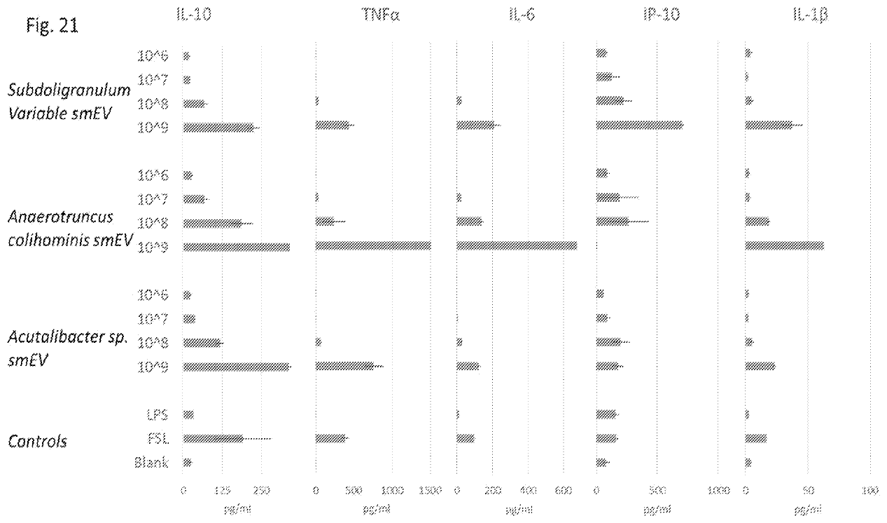

The results are shown in FIG. 21.

smEVs were harvested from bioreactor-grown bacteria from Acutalibacter sp. Strain A, Anaerotruncus colihominis Strain A, and Subdoligranulum variabile Strain A, with yields above 3E13 particles per liter. These isolates from this family exhibit different cytokine profiles from smEVs previously isolated.

Note the strong IP-10 stimulation from Subdoligranulum variable smEVs.

Anaerotruncus colihominis smEVs were robust cytokine inducers. Note that IP-10 expression was diminished at the highest concentration, likely due to overstimulation bordering on toxicity (as evidenced by the high IL-1b levels).

Acutalibacter sp. smEVs strongly stimulate IL-10 and the other cytokines to a lesser extent which is more similar to the Oscillospiraceae smEVs characterized previously such as smEVs from Harryflintia acetispora.

### Example 43: Acutalibactersp. Strain A, Anaerotruncus colihominis Strain A, and Subdoligranulum Variabile Strain A smEVs in a Mouse Model of delayed-Type Hypersensitivity (DTH)

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were orally gavaged daily with smEVs or dosed intraperitoneally with dexamethasone at 1 mg/kg from days 5-8. After dosing on day 8, mice were anaesthetized with isoflurane, left ears were measured for baseline measurements with Fowler calipers and the mice were challenged intradermally with KLH in saline (10 μl) in the left ear and ear thickness measurements were taken at 24 hours.

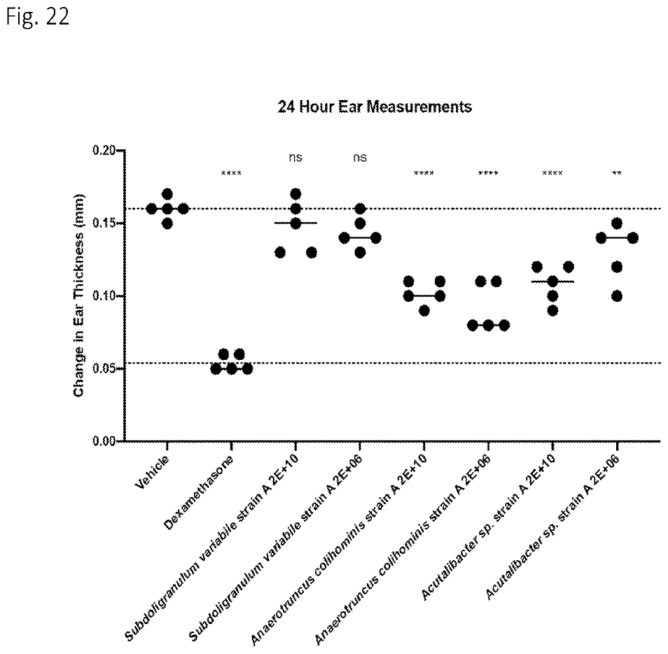

The 24 hour ear measurement results are shown in FIG. 22. smEVs made from Acutalibacter sp. strain A, Anaerotruncus colihominis strain A and Subdoligranulum variabile strain A were compared at two doses, 2E+10 and 2E+06 (based on particles per dose). The smEVs made from Acutalibacter sp. strain A and Anaerotruncus colihominis strain A were efficacious at both doses, showing decreased ear inflammation 24 hours after challenge. The smEVs made from Subdoligranulum variabile strain A were not efficacious at either dose.

### Example 44: Subdoligranulum variabile Strain A smEVs in a Mouse Model of Delayed-Type Hypersensitivity (DTH)

Female 5 week old C57BL/6 mice were purchased from Taconic Biosciences and acclimated at avivarium for one week. Mice were primed with an emulsion of KLH and CFA (1:1) by subcutaneous immunization on day 0. Mice were orally gavaged daily with smEVs or dosed intraperitoneally with dexamethasone at 1 mg/kg from days 5-8. After dosing on day 8, mice were anaesthetized with isoflurane, left ears were measured for baseline measurements with Fowler calipers and the mice were challenged intradermally with KLH in saline (10 μl) in the left car and ear thickness measurements were taken at 24 hours.

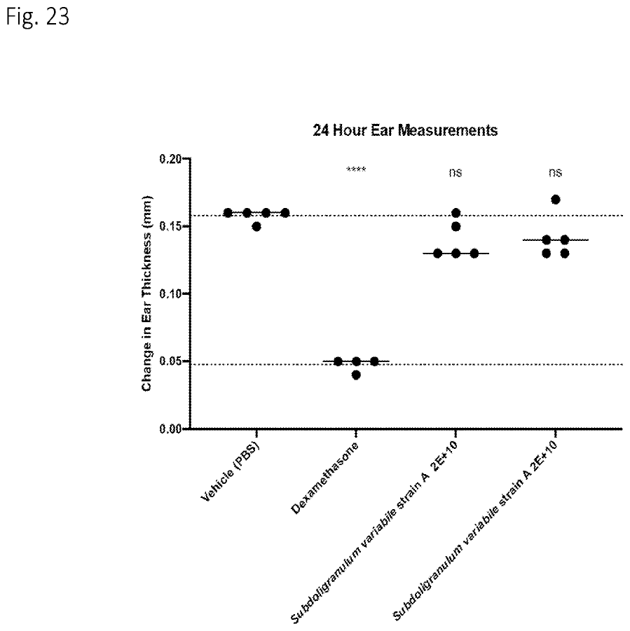

The 24 hour ear measurement results are shown in FIG. 23. Two batches of EVs made from Subdoligranulum variabile strain A were compared at 2E+10 (based on particles per dose). Neither batch of the smEVs made from Subdoligranulum variable strain A was efficacious.

### Example 45: Anaerotruncus colihominis Strain A smEVs in a Mouse Model of Delayed-Type Hypersensitivity (DTH)

Female 5 week old C57BL/6 mice were purchased from Taconic Biosciences and acclimated at avivarium for one week. Mice were primed with an emulsion of KLH and CFA (1:1) by subcutaneous immunization on day 0. Mice were orally gavaged daily with smEVs or dosed intraperitoneally with dexamethasone at 1 mg/kg from days 5-8. After dosing on day 8, mice were anaesthetized with isoflurane, left ears were measured for baseline measurements with Fowler calipers and the mice were challenged intradermally with KLH in saline (10 μl) in the left ear and ear thickness measurements were taken at 24 hours.

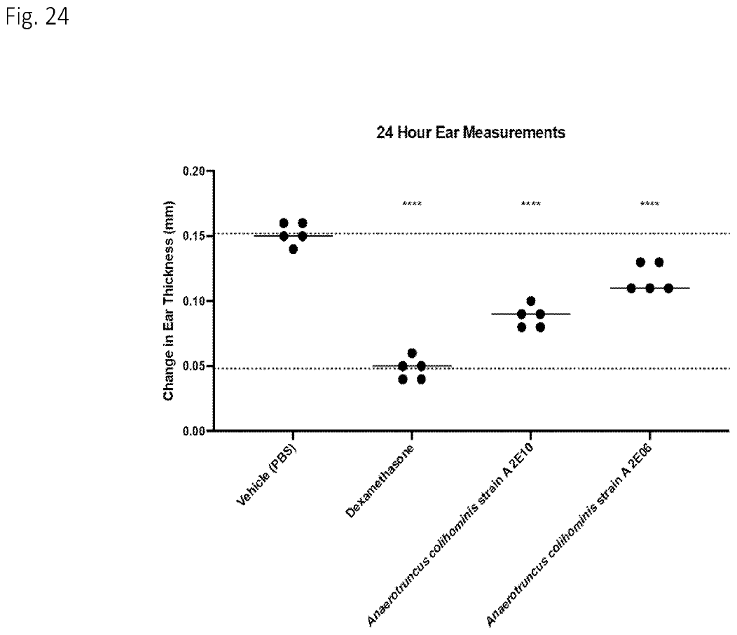

The 24 hour ear measurement results are shown in FIG. 24. smEVs made from Anaerotruncus colihominis strain A were compared at two doses 2E+10 and 2E+06 (based on particles per dose). Both of these doses were efficacious compared to Vehicle and there was a dose response trend seen.

### Example 46: Anaerotruncus colihominis Strain A smEVs have Potent Human TLR2 and TLR5 Agonist Activity

Anaerotruncus colihominis strain A smEVs stimulate TLR2/6 heterodimers over TLR 1/2 but have low Emax. Anaerotruncus colihominis strain A smEVs have medium TLR5 agonism with higher Emax than other TLRs.

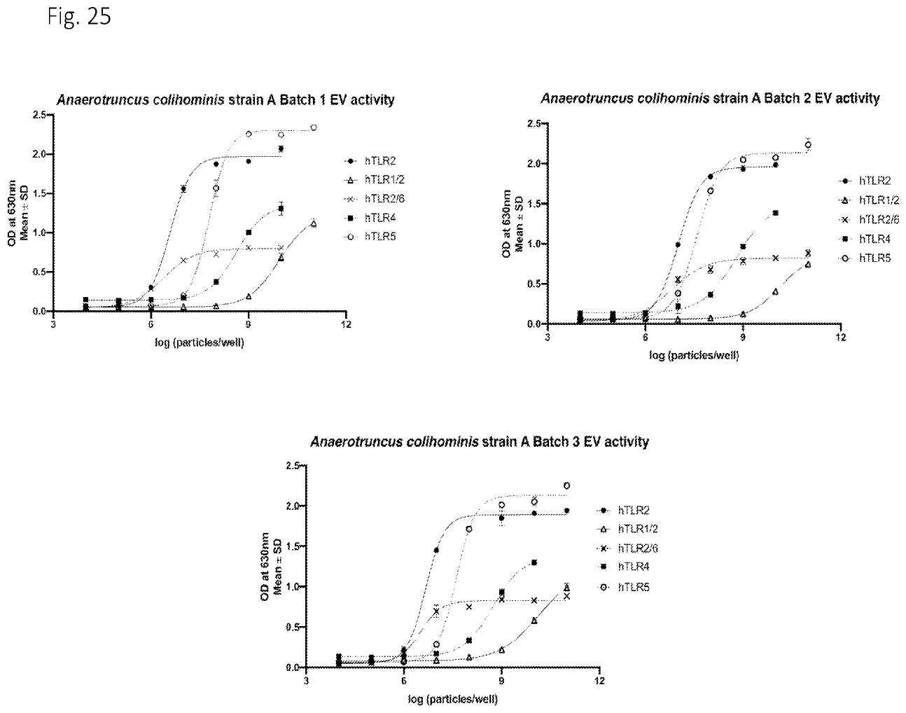

HEK293-SEAP reporter cells (Invivogen) expressing human TLR1, TLR2, and TLR6 combinations and human TLR4 and TLR5 were plated at a final concentration of 20,000 cells per well in 96 well plates and cultured in appropriate selection media. After 48 hours, selection media was washed out and replaced with complete media, and Anaerotruncus colihominis strain A smEVs were added at the indicated concentrations per well. Cells were cultured in the presence of the smEVs for 24 hours. Supernatant was collected and incubated with HEK-Blue reagent (Invivogen) for 30 minutes, followed by reading absorbance at OD 630 nm for stimulation of TLR2 heterodimers, TLR4 and TLR5 cells. The results are shown in FIG. 25.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims

What is claimed is:

1. A pharmaceutical composition comprising isolated pharmaceutical composition microbial extracellular vesicles (mEVs) from Oscillospiraceae bacteria (e.g., a therapeutically effective amount of mEVs from Oscillospiraceae bacteria).

2. The pharmaceutical composition of claim 1, wherein at least 75%, at least 80%, at least 85%, at least 90%, at least 95%, or at least 99% of the pharmaceutical composition is mEVs.

3. A pharmaceutical composition comprising isolated mEVs or a therapeutically effective amount for the treatment of a cancer.

4. A pharmaceutical composition comprising isolated mEVs or a therapeutically effective amount for the treatment of a dysbiosis.

5. A pharmaceutical composition comprising isolated mEVs or a therapeutically effective amount for the treatment of an autoimmune disease.

6. A pharmaceutical composition comprising isolated mEVs or a therapeutically effective amount for the treatment of an inflammatory disease.

7. A pharmaceutical composition comprising isolated mEVs or a therapeutically effective amount for the treatment of a metabolic disease.

8. The pharmaceutical composition of any one of claims 1 to 7, wherein the composition comprises mEVs from a strain of Oscillospiraceae bacteria comprising at least 99% genomic, 16S and/or CRISPR sequence identity to the nucleotide sequence of the Faecalibacterium prausnitzii Strain A (ATCC Deposit Number PTA-126792).

9. The pharmaceutical composition of any one of claims 1 to 7, wherein the composition comprises mEVs from the Faecalibacterium prausnitrzii Strain A (ATCC Deposit Number PTA-126792).

10. The pharmaceutical composition of any one of claims 1 to 7, wherein the composition comprises mEVs from a strain of Oscillospiraceae bacteria comprising at least 99% genomic, 16S and/or CRISPR sequence identity to the nucleotide sequence of the Fournierella massiliensis Strain A (ATCC Deposit Number PTA-126696).

11. The pharmaceutical composition of any one of claims 1 to 7, wherein the composition comprises mEVs from the Fournierella massiliensis Strain A (ATCC Deposit Number PTA-126696).

12. The pharmaceutical composition of any one of claims 1 to 7, wherein the composition comprises mEVs from a strain of Oscillospiraceae bacteria comprising at least 99% genomic, 16S and/or CRISPR sequence identity to the nucleotide sequence of the Harryflintia acetispora Strain A (ATCC Deposit Number PTA-126694).

13. The pharmaceutical composition of any one of claims 1 to 7, wherein the composition comprises mEVs from the Harryflintia acetispora Strain A (ATCC Deposit Number PTA-126694).

14. The pharmaceutical composition of any one of claims 1 to 7, wherein the composition comprises mEVs from a strain of Oscillospiraceae bacteria comprising at least 99% genomic, 16S and/or CRISPR sequence identity to the nucleotide sequence of the Agathobaculum sp. Strain A (ATCC Deposit Number PTA-125892).

15. The pharmaceutical composition of any one of claims 1 to 7, wherein the composition comprises mEVs from the Agathobaculum sp. Strain A (ATCC Deposit Number PTA-125892).

16. The pharmaceutical composition of any one of claims 1 to 7, wherein the composition comprises mEVs from a strain of Oscillospiraceae bacteria comprising at least 99% genomic, 16S and/or CRISPR sequence identity to the nucleotide sequence of the Acutalibactersp. Strain A (ATCC Deposit Number PTA-127006).

17. The pharmaceutical composition of any one of claims 1 to 7, wherein the composition comprises mEVs from the Acutalibacter sp. Strain A (ATCC Deposit Number PTA-127006).

18. The pharmaceutical composition of any one of claims 1 to 7, wherein the composition comprises mEVs from a strain of Oscillospiraceae bacteria comprising at least 99% genomic, 16S and/or CRISPR sequence identity to the nucleotide sequence of the Anaerotruncus colihominis Strain A (ATCC Deposit Number PTA-127005).

19. The pharmaceutical composition of any one of claims 1 to 7, wherein the composition comprises mEVs from the Anaerotruncus colihominis Strain A (ATCC Deposit Number PTA-127005).

20. The pharmaceutical composition of any one of claims 1 to 7, wherein the composition comprises mEVs from a strain of Oscillospiraceae bacteria comprising at least 99% genomic, 16S and/or CRISPR sequence identity to the nucleotide sequence of the Subdoligranulum variabile Strain A (ATCC Deposit Number PTA-127004).

21. The pharmaceutical composition of any one of claims 1 to 7, wherein the composition comprises mEVs from the Subdoligranulum variabile Strain A (ATCC Deposit Number PTA-127004).

22. The pharmaceutical composition of any one of claims 1 to 21, wherein the composition activates innate antigen presenting cells.

23. The pharmaceutical composition of any one of claims 1 to 21, wherein the composition has one or more beneficial immune effects outside the gastrointestinal tract, e.g., when orally administered.

24. The pharmaceutical composition of any one of claims 1 to 21, wherein the composition modulates immune effects outside the gastrointestinal tract in the subject, e.g., when orally administered.

25. The pharmaceutical composition of any one of claims 1 to 24, wherein the mEVs are produced from a high yield strain.

26. The pharmaceutical composition of claim 25, wherein the high yield strain produces at least 3×10¹³ mEVs per liter from a bioreactor-grown culture.

27. The pharmaceutical composition of any one of claims 1 to 26, wherein the mEVs comprise pmEVs and the pmEVs are produced from bacteria that have been gamma irradiated, UV irradiated, heat inactivated, acid treated or oxygen sparged.

28. The pharmaceutical composition of any one of claims 1 to 26, wherein the mEVs comprise pmEVs and the pmEVs are produced from live bacteria.

29. The pharmaceutical composition of any one of claims 1 to 28, wherein the composition comprises secreted mEVs (smEVs).

30. The pharmaceutical composition of any one of claims 1 to 28, wherein the composition comprises processed mEVs (pmEVs).

31. The pharmaceutical composition of any one of claims 1 to 30, wherein the mEVs are lyophilized (e.g., the lyophilized product further comprises a pharmaceutically acceptable excipient).

32. The pharmaceutical composition of any one of claims 1 to 31, wherein the mEVs are gamma irradiated.

33. The pharmaceutical composition of any one of claims 1 to 31, wherein the mEVs are UV irradiated.

34. The pharmaceutical composition of any one of claims 1 to 31, wherein the mEVs are heat inactivated (e.g., at 50° C. for two hours or at 90° C. for two hours).

35. The pharmaceutical composition of any one of claims 1 to 31, wherein the mEVs are acid treated.

36. The pharmaceutical composition of any one of claims 1 to 31, wherein the mEVs are oxygen sparged (e.g., at 0.1 vvm for two hours).

37. The pharmaceutical composition of any one of claims 1 to 36, wherein the dose of mEVs is about 2×10⁶ to about 2×10¹⁶ particles (e.g., wherein particle count is determined by NTA (nanoparticle tracking analysis)).

38. The pharmaceutical composition of any one of claims 1 to 36, wherein the dose of mEVs is about 5 mg to about 900 mg total protein (e.g., wherein total protein is determined by Bradford assay or BCA assay).

39. The pharmaceutical composition of any one of claims 1 to 38, wherein the pharmaceutical composition comprises a solid dose form.

40. The pharmaceutical composition of claim 39, wherein the solid dose form comprises a tablet, a minitablet, a capsule, a pill, or a powder, or a combination of the foregoing.

41. The pharmaceutical composition of claim 39 or 40, wherein the solid dose form further comprises a pharmaceutically acceptable excipient.

42. The pharmaceutical composition of any one of claims 39 to 41, wherein the solid dose form comprises an enteric coating.

43. The pharmaceutical composition of any one of claims 39 to 42, wherein the solid dose form is for oral administration.

44. The pharmaceutical composition of any one of claims 1 to 38, wherein the pharmaceutical composition comprises a suspension.

45. The pharmaceutical composition of claim 44, wherein the suspension is for oral administration (e.g., the suspension comprises PBS, and optionally, sucrose or glucose).

46. The pharmaceutical composition of claim 44, wherein the suspension is for intravenous administration (e.g., the suspension comprises PBS).

47. The pharmaceutical composition of claim 44, wherein the suspension is for intraperitoneal administration (e.g., the suspension comprises PBS).

48. The pharmaceutical composition of claim 44, wherein the suspension is for intratumoral administration (e.g., the suspension comprises PBS).

49. The pharmaceutical composition of any one of claims 44 to 48, wherein the suspension further comprises a pharmaceutically acceptable excipient.

50. The pharmaceutical composition of any one of claims 44 to 49, wherein the suspension further comprises a buffer (e.g., PBS).

51. The pharmaceutical composition of any one of claims 1 to 50, wherein the composition comprises a bacterial strain listed in Table 2.

52. The pharmaceutical composition of any one of claims 1 to 50, wherein the composition further comprises one or more additional therapeutic agents.

53. The pharmaceutical composition of any one of claims 1 to 52 for use in treating a disease (e.g., a cancer, an autoimmune disease, an inflammatory disease, a dysbiosis, and/or a metabolic disease).

54. Use of a pharmaceutical composition of any one of claims 1 to 52 for the preparation of a medicament for the treatment of a disease (e.g., a cancer, an autoimmune disease, an inflammatory disease, a dysbiosis, and/or a metabolic disease).

55. A method of treating a subject (e.g., human), comprising administering to the subject a pharmaceutical composition of any one of claims 1-52.

56. The method of claim 55, wherein the subject is in need of treatment for a cancer.

57. The method of claim 55, wherein the subject is in need of treatment for an inflammatory disease.

58. The method of claim 55, wherein the subject is in need of treatment for a dysbiosis.

59. The method of claim 55, wherein the subject is in need of treatment for an autoimmune disease.

60. The method of claim 55, wherein the subject is in need of treatment for a metabolic disease.

61. The method of any one of claims 55 to 60, wherein the pharmaceutical composition is administered in combination with an additional therapeutic agent.

62. The method of any one of claims 55 to 61, wherein the pharmaceutical composition is administered intravenously.

63. The method of any one of claims 55 to 61, wherein the pharmaceutical composition is administered intratumorally.

64. The method of any one of claims 55 to 61, wherein the pharmaceutical composition is administered subtumorally.

65. The method of any one of claims 55 to 61, wherein the pharmaceutical composition is administered by injection, e.g., subcutaneous, intradermal, or intraperitoneal injection.

66. The method of any one of claims 55 to 65, wherein the composition further comprises one or more additional therapeutic agents.

67. The method of any one of claims 55 to 66, wherein the pharmaceutical composition is administered orally.

68. The method of any one of claims 55 to 67, wherein the dose of mEVs in the pharmaceutical composition is about 2×10⁶ to about 2×10¹⁶ particles (e.g., wherein particle count is determined by NTA (nanoparticle tracking analysis)).

69. The method of any one of claims 55 to 67, wherein the dose of mEVs in the pharmaceutical composition is 5 mg to about 900 mg total protein (e.g., wherein total protein is determined by Bradford assay or BCA assay).

70. The method of any one of claims 55 to 68, wherein the pharmaceutical composition is administered once a day.

71. The method of any one of claims 55 to 68, wherein the pharmaceutical composition is administered twice a day.

72. The method of any one of claims 55 to 68, wherein the pharmaceutical composition is formulated for a daily dose.

73. The method of any one of claims 55 to 68, wherein the pharmaceutical composition is formulated for twice a day dose, wherein each dose is half of the daily dose.

74. A method for preparing a pharmaceutical composition comprising mEVs (e.g., a therapeutically effective amount thereof) of any one of claims 1-52 in a suspension, the method comprising: combining mEVs with a pharmaceutically acceptable buffer (e.g., PBS); thereby preparing the pharmaceutical composition.

75. The method of claim 74, wherein the suspension further comprises sucrose or glucose.

76. The method of claim 74 or 75, wherein the suspension is for oral administration.

77. The method of claim 74 or 75, wherein the suspension is for intravenous administration.

78. The method of claim 74 or 75, wherein the suspension is for intraperitoneal administration.

79. The method of claim 74 or 75, wherein the suspension is for intratumoral administration.

80. The method of any one of claims 74 to 79, wherein the suspension further comprises a pharmaceutically acceptable excipient.

81. The method of any one of claims 74 to 80, wherein the suspension further comprises a buffer (e.g., PBS).

82. The method of any one of claims 74 to 81, wherein the composition further comprises one or more additional therapeutic agents.

83. The method of any one of claims 74 to 82, wherein the pharmaceutical composition is administered orally.

84. The method of any one of claims 74 to 82, wherein the pharmaceutical composition is administered intravenously.

85. The method of any one of claims 74 to 82, wherein the pharmaceutical composition is administered intratumorally.

86. The method of any one of claims 74 to 82, wherein the pharmaceutical composition is administered subtumorally.

87. The method of any one of claims 74 to 82, wherein the pharmaceutical composition is administered by injection, e.g., subcutaneous, intradermal, or intraperitoneal injection.

88. The method of any one of claims 74 to 87, wherein the dose of mEVs in the pharmaceutical composition is about 2×10⁶ to about 2×10¹⁶ particles (e.g., wherein particle count is determined by NTA (nanoparticle tracking analysis)).

89. The method of any one of claims 74 to 87, wherein the dose of mEVs in the pharmaceutical composition is 5 mg to about 900 mg total protein (e.g., wherein total protein is determined by Bradford assay or BCA assay).

90. A pharmaceutical composition prepared by the method of any one of claims 74 to 89.

91. A method for preparing a pharmaceutical composition comprising mEVs (e.g., a therapeutically effective amount thereof) in a solid dose form, the method comprising:
a) combining mEVs of any one of claims 1-52 with a pharmaceutically acceptable excipient; and
b) compressing the mEVs and pharmaceutically acceptable excipient; thereby preparing the pharmaceutical composition.

92. The method of claim 91, wherein the method further comprises enterically coating the solid dose form.

93. The method of claim 91 or 92, wherein the solid dose form comprises a tablet, a minitablet, a capsule, a pill, or a powder, or a combination of the foregoing.

94. The method of any one of claims 91 to 93, wherein the composition further comprises one or more additional therapeutic agents.

95. The method of any one of claims 91 to 94, wherein the pharmaceutical composition is administered orally.

96. The method of any one of claims 91 to 95, wherein the dose of mEVs in the pharmaceutical composition is about 2×10⁶ to about 2×10¹⁶ particles (e.g., wherein particle count is determined by NTA (nanoparticle tracking analysis)).

97. The method of any one of claims 91 to 95, wherein the dose of mEVs in the pharmaceutical composition is 5 mg to about 900 mg total protein (e.g., wherein total protein is determined by Bradford assay or BCA assay).

98. A pharmaceutical composition prepared by the method of any one of claims 91 to 97.
